SEQUENCE LISTING (42 sequences)

SEQ ID NO: 1 - protein, 32 aa, Listeria monocytogenes
KENSISSMAPPASPPASPKTPIEKKHADEIDK

SEQ ID NO: 2 - protein, 19 aa, Listeria monocytogenes
KENSISSMAPPASPPASPK

SEQ ID NO: 3 - protein, 14 aa, Listeria monocytogenes
KTEEQPSEVNTGPR

SEQ ID NO: 4 - protein, 28 aa, Listeria monocytogenes
KASVTDTSEGDLDSSMQSADESTPQPLK

SEQ ID NO: 5 - protein, 20 aa, Listeria monocytogenes
KNEEVNASDFPPPPTDEELR

SEQ ID NO: 6 - protein, 33 aa, Listeria monocytogenes
RGGIPTSEEFSSLNSGDFTDDENSETTEEEIDR

SEQ ID NO: 7 - protein, 19 aa, Listeria seeligeri
RSEVTISPAETPESPPATP

SEQ ID NO: 8 - protein, 17 aa, Streptococcus pyogenes
KQNTASTETTTTNEQPK

SEQ ID NO: 9 - protein, 17 aa, Streptococcus equisimilis
KQNTANTETTTTNEQPK

SEQ ID NO: 10 - protein, 441 aa, Artificial Sequence
  N-terminal LLO fragment
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 11 - protein, 416 aa, Artificial Sequence
  LLO fragment
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 12 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 13 - protein, 416 aa, Artificial Sequence; identical to SEQ ID NO: 11
  LLO protein

SEQ ID NO: 14 - protein, 9 aa, Artificial Sequence
  HLA-A2 suvivin epitope
LMLGEFLKL

SEQ ID NO: 15 - protein, 390 aa, Artificial Sequence
  N-terminal fragment of an ActA protein
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 16 - DNA, 1170 bp, Listeria monocytogenes
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 17 - protein, 390 aa, Listeria monocytogenes
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRP

SEQ ID NO: 18 - DNA, 1170 bp, Listeria monocytogenes
atgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccgatgcttctcggttttaatgctcctactccatcggaaccgagctcattcgaatttccgccgccacctacggatgaagagttaagacttgctttgccagagacgccaatgcttcttggttttaatgctcctgctacatcggaaccgagctcattcgaatttccaccgcctccaacagaagatgaactagaaattatgcgggaaacagcaccttcgctagattctagttttacaagcggggatttagctagtttgagaagtgctattaatcgccatagcgaaaatttctctgatttcccactaatcccaacagaagaagagttgaacgggagaggcggtagacca

SEQ ID NO: 19 - DNA, 1256 bp, Listeria monocytogenes
gcgccaaatcattggttgattggtgaggatgtctgtgtgcgtgggtcgcgagatgggcgaataagaagcattaaagatcctgacaaatataatcaagcggctcatatgaaagattacgaatcgcttccactcacagaggaaggcgactggggcggagttcattataatagtggtatcccgaataaagcagcctataatactatcactaaacttggaaaagaaaaaacagaacagctttattttcgcgccttaaagtactatttaacgaaaaaatcccagtttaccgatgcgaaaaaagcgcttcaacaagcagcgaaagatttatatggtgaagatgcttctaaaaaagttgctgaagcttgggaagcagttggggttaactgattaacaaatgttagagaaaaattaattctccaagtgatattcttaaaataattcatgaatattttttcttatattagctaattaagaagataactaactgctaatccaatttttaacggaacaaattagtgaaaatgaaggccgaattttccttgttctaaaaaggttgtattagcgtatcacgaggagggagtataagtgggattaaacagatttatgcgtgcgatgatggtggttttcattactgccaattgcattacgattaaccccgacgtcgacccatacgacgttaattcttgcaatgttagctattggcgtgttctctttaggggcgtttatcaaaattattcaattaagaaaaaataattaaaaacacagaacgaaagaaaaagtgaggtgaatgatatgaaattcaaaaaggtggttctaggtatgtgcttgatcgcaagtgttctagtctttccggtaacgataaaagcaaatgcctgttgtgatgaatacttacaaacacccgcagctccgcatgatattgacagcaaattaccacataaacttagttggtccgcggataacccgacaaatactgacgtaaatacgcactattggctttttaaacaagcggaaaaaatactagctaaagatgtaaatcatatgcgagctaatttaatgaatgaacttaaaaaattcgataaacaaatagctcaaggaatatatgatgcggatcataaaaatccatattatgatactagtacatttttatctcatttttataatcctgatagagataatacttatttgccgggttttgctaatgcgaaaataacaggagcaaagtatttcaatcaatcggtgactgattaccgagaagggaa

SEQ ID NO: 20 - protein, 140 aa, Mus musculus
MGAPALPQIWQLYLKNYRIATFKNWPFLEDCACAPERMAEAGFIHCPTENEPDLAQCFFCFKELEGWEPDDNPIEEHRKHSPGCAFLTVKKQMEELTVSEFLKLDRQRAKNKIAKETNNKQKEFEETAKTTRQSIEQLAA

SEQ ID NO: 21 - protein, 141 aa, Homo sapiens
GAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 22 - protein, 421 aa, Mus musculus
GGGAGCTCCGGCGCTGCCCCAGATCTGGCAGCTGTACCTCAAGAACTACCGCATCGCCACCTTCAAGAACTGGCCCTTCCTGGAGGACTGCGCCTGCGCCCCAGAGCGAATGGCGGAGGCTGGCTTCATCCACTGCCCTACCGAGAACGAGCCTGATTTGGCCCAGTGTTTTTTCTGCTTTAAGGAATTGGAAGGCTGGGAACCCGATGACAACCCGATAGAGGAGCATAGAAAGCACTCCCCTGGCTGCGCCTTCCTCACTGTCAAGAAGCAGATGGAAGAACTAACCGTCAGTGAATTCTTGAAACTGGACAGACAGAGAGCCAAGAACAAAATTGCAAAGGAGACCAACAACAAGCAAAAAGAGTTTGAAGAGACTGCAAAGACTACCCGTCAGTCAATTGAGCAGCTGGCTGCCTAA

SEQ ID NO: 23 - protein, 426 aa, Homo sapiens
GGTGCCCCGACGTTGCCCCCTGCCTGGCAGCCCTTTCTCAAGGACCACCGCATCTCTACATTCAAGAACTGGCCCTTCTTGGAGGGCTGCGCCTGCGCCCCGGAGCGGATGGCCGAGGCTGGCTTCATCCACTGCCCCACTGAGAACGAGCCAGACTTGGCCCAGTGTTTCTTCTGCTTCAAGGAGCTGGAAGGCTGGGAGCCAGATGACGACCCCATAGAGGAACATAAAAAGCATTCGTCCGGTTGCGCTTTCCTTTCTGTCAAGAAGCAGTTTGAAGAATTAACCCTTGGTGAATTTTTGAAACTGGACAGAGAAAGAGCCAAGAACAAAATTGCAAAGGAAACCAACAATAAGAAGAAAGAATTTGAGGAAACTGCGAAGAAAGTGCGCCGTGCCATCGAGCAGCTGGCTGCCATGGATTGA

SEQ ID NO: 24 - DNA, 1107 bp, Listeria monocytogenes
atggtgacaggctggcatcgtccaacatggattgaaatagaccgcgcagcaattcgcgaaaatataaaaaatgaacaaaataaactcccggaaagtgtcgacttatgggcagtagtcaaagctaatgcatatggtcacggaattatcgaagttgctaggacggcgaaagaagctggagcaaaaggtttctgcgtagccattttagatgaggcactggctcttagagaagctggatttcaagatgactttattcttgtgcttggtgcaaccagaaaagaagatgctaatctggcagccaaaaaccacatttcacttactgtttttagagaagattggctagagaatctaacgctagaagcaacacttcgaattcatttaaaagtagatagcggtatggggcgtctcggtattcgtacgactgaagaagcacggcgaattgaagcaaccagtactaatgatcaccaattacaactggaaggtatttacacgcattttgcaacagccgaccagctagaaactagttattttgaacaacaattagctaagttccaaacgattttaacgagtttaaaaaaacgaccaacttatgttcatacagccaattcagctgcttcattgttacagccacaaatcgggtttgatgcgattcgctttggtatttcgatgtatggattaactccctccacagaaatcaaaactagcttgccgtttgagcttaaacctgcacttgcactctataccgagatggttcatgtgaaagaacttgcaccaggcgatagcgttagctacggagcaacttatacagcaacagagcgagaatgggttgcgacattaccaattggctatgcggatggattgattcgtcattacagtggtttccatgttttagtagacggtgaaccagctccaatcattggtcgagtttgtatggatcaaaccatcataaaactaccacgtgaatttcaaactggttcaaaagtaacgataattggcaaagatcatggtaacacggtaacagcagatgatgccgctcaatatttagatacaattaattatgaggtaacttgtttgttaaatgagcgcatacctagaaaatacatccattag

SEQ ID NO: 25 - protein, 368 aa, Listeria monocytogenes
MVTGWHRPTWIEIDRAAIRENIKNEQNKLPESVDLWAVVKANAYGHGIIEVARTAKEAGAKGFCVAILDEALALREAGFQDDFILVLGATRKEDANLAAKNHISLTVFREDWLENLTLEATLRIHLKVDSGMGRLGIRTTEEARRIEATSTNDHQLQLEGIYTHFATADQLETSYFEQQLAKFQTILTSLKKRPTYVHTANSAASLLQPQIGFDAIRFGISMYGLTPSTEIKTSLPFELKPALALYTEMVHVKELAPGDSVSYGATYTATEREWVATLPIGYADGLIRHYSGFHVLVDGEPAPIIGRVCMDQTIIKLPREFQTGSKVTIIGKDHGNTVTADDAAQYLDTINYEVTCLLNERIPRKYIH

SEQ ID NO: 26 - DNA, 870 bp, Listeria monocytogenes
atgaaagtattagtaaataaccatttagttgaaagagaagatgccacagttgacattgaagaccgcggatatcagtttggtgatggtgtatatgaagtagttcgtctatataatggaaaattctttacttataatgaacacattgatcgcttatatgctagtgcagcaaaaattgacttagttattccttattccaaagaagagctacgtgaattacttgaaaaattagttgccgaaaataatatcaatacagggaatgtctatttacaagtgactcgtggtgttcaaaacccacgtaatcatgtaatccctgatgatttccctctagaaggcgttttaacagcagcagctcgtgaagtacctagaaacgagcgtcaattcgttgaaggtggaacggcgattacagaagaagatgtgcgctggttacgctgtgatattaagagcttaaaccttttaggaaatattctagcaaaaaataaagcacatcaacaaaatgctttggaagctattttacatcgcggggaacaagtaacagaatgttctgcttcaaacgtttctattattaaagatggtgtattatggacgcatgcggcagataacttaatcttaaatggtatcactcgtcaagttatcattgatgttgcgaaaaagaatggcattcctgttaaagaagcggatttcactttaacagaccttcgtgaagcggatgaagtgttcatttcaagtacaactattgaaattacacctattacgcatattgacggagttcaagtagctgacggaaaacgtggaccaattacagcgcaacttcatcaatattttgtagaagaaatcactcgtgcatgtggcgaattagagtttgcaaaataa

SEQ ID NO: 27 - protein, 289 aa, Listeria monocytogenes
MKVLVNNHLVEREDATVDIEDRGYQFGDGVYEVVRLYNGKFFTYNEHIDRLYASAAKIDLVIPYSKEELRELLEKLVAENNINTGNVYLQVTRGVQNPRNHVIPDDFPLEGVLTAAAREVPRNERQFVEGGTAITEEDVRWLRCDIKSLNLLGNILAKNKAHQQNALEAILHRGEQVTECSASNVSIIKDGVLWTHAADNLILNGITRQVIIDVAKKNGIPVKEADFTLTDLREADEVFISSTTIEITPITHIDGVQVADGKRGPITAQLHQYFVEEITRACGELEFAK

SEQ ID NO: 28 - DNA, 6523 bp, Artificial Sequence
  Plasmid pAdv142
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccctaacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 29 - DNA, 36 bp, Artificial Sequence
  Adv271-actAF1 primer
cggaattcggatccgcgccaaatcattggttgattg

SEQ ID NO: 30 - DNA, 37 bp, Artificial Sequence
  Adv272-actAR1 primer
gcgagtcgacgtcggggttaatcgtaatgcaattggc

SEQ ID NO: 31 - DNA, 35 bp, Artificial Sequence
  Adv273-actAF2 primer
gcgagtcgacccatacgacgttaattcttgcaatg

SEQ ID NO: 32 - DNA, 39 bp, Artificial Sequence
  Adv274-actAR2 primer
gatactgcagggatccttcccttctcggtaatcagtcac

SEQ ID NO: 33 - DNA, 19 bp, Artificial Sequence
  primer 3 which binds externally to actA region
tgggatggccaagaaattc

SEQ ID NO: 34 - DNA, 22 bp, Artificial Sequence
  Primer 4 which binds externally to actA region
ctaccatgtcttccgttgcttg

SEQ ID NO: 35 - protein, 10 aa, Artificial Sequence
  PSA H-2Db peptide
HCIRNKSVIL

SEQ ID NO: 36 - protein, 9 aa, Artificial Sequence
  HPV16 E7 H-2Db
RAHYNIVTF

SEQ ID NO: 37 - DNA, 26 bp, Artificial Sequence
  Primer for amplifying mouse survivin
atctcgagggagctccggcgctgccc

SEQ ID NO: 38 - DNA, 26 bp, Artificial Sequence
  Primer for amplifying mouse survivin
atcccgggttaggcagccagctgctc

SEQ ID NO: 39 - DNA, 26 bp, Artificial Sequence
  Primer for amplifying human survivin
atctcgagggtgccccgacgttgccc

SEQ ID NO: 40 - DNA, 27 bp, Artificial Sequence
  Primer for amplifying human survivin
atcccgggtcaatccatggcagccagc

SEQ ID NO: 41 - DNA, 6235 bp, Artificial Sequence
  Human Survivin DNA sequence in plasmid pAdv265.5
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagggtgccccgacgttgccccctgcctggcagccctttctcaaggaccaccgcatctctacattcaagaactggcccttcttggagggctgcgcctgcgccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattgacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 42 - protein, 11 aa, Listeria monocytogenes
ECTGLAWEWWR

(19) United States
(12) Patent Application Publication
(10) Pub. No.: US 2017/0246273 A1
(43) Pub. Date: Aug. 31, 2017

(54) RECOMBINANT LISTERIA STRAIN EXPRESSING HETEROLOGOUS ANTIGEN FUSION PROTEINS AND METHODS OF USE THEREOF

(71) Applicants: Advaxis, Inc., Princeton, NJ (US); City of Hope, Duarte, CA (US)

(72) Inventors: Anu Wallecha, Yardley, PA (US); Robert Petit, Newtown (Wrightstown), PA (US); Joshua Ellenhorn, Berverly Hills, CA (US); Don J. Diamond, Glendora, CA (US)

(21) Appl. No.: 15/325,955

(22) PCT Filed: Jul. 17, 2015
(86) PCT No.: PCT/US15/40911

§ 371 Date: Jan. 12, 2017

Related U.S. Application Data
(60) Provisional application No. 62/026,085, filed on Jul. 18, 2014.

(51) Int. Cl. A61K 39/00; C12N 9/90; A61K 39/385; C12N 15/85; C07K 14/195; C07K 14/47

(52) U.S. Cl. CPC A61K 39/0011; C07K 14/195; C07K 14/4703; A61K 39/385; C12N 15/85; C12Y 501/01001; C12N 9/90; A61K 2039/6037; A61K 2039/523; A61K 2039/522; C07K 2319/55; C07K 2319/40; A61K 2039/572; C12N 2800/107; C12N 2820/55

(57) ABSTRACT

Disclosed herein are recombinant nucleic acids encoding tumor antigens fused to immunogenic polypeptides and recombinant Listeria strains comprising the same, methods of preparing same, and methods of inducing an immune response, and treating, inhibiting, or suppressing cancer or tumors comprising administering same.

### FIELD OF INVENTION

Disclosed herein are recombinant nucleic acids encoding tumor antigens fused to immunogenic polypeptides and recombinant Listeria strains comprising the same, methods of preparing same, and methods of inducing an immune response, and treating, inhibiting, or suppressing cancer or tumors comprising administering same.

### BACKGROUND OF THE INVENTION

A great deal of pre-clinical evidence and early clinical trial data suggests that the anti-tumor capabilities of the immune system can be harnessed to treat patients with established cancers. The vaccine strategy takes advantage of tumor antigens associated with various types of cancers Immunizing with live vaccines such as viral or bacterial vectors expressing a tumor-associated antigen is one strategy for eliciting strong CTL responses against tumors.

Listeria monocytogenes (Lm) is a gram positive, facultative intracellular bacterium that has direct access to the cytoplasm of antigen presenting cells, such as macrophages and dendritic cells, largely due to the pore-forming activity of listeriolysin-O (LLO). LLO is secreted by Lm following engulfment by the cells and perforates the phagolysosomal membrane, allowing the bacterium to escape the vacuole and enter the cytoplasm. LLO is very efficiently presented to the immune system via MHC class I molecules. Furthermore, Lm-derived peptides also have access to MHC class II presentation via the phagolysosome.

Survivin, an inhibitor of apoptosis protein is highly expressed in most cancers and associated with chemotherapy resistance, increased tumor recurrence, and shorter patient survival. There is a continuing need to find therapies that are effective against cancer. Due to its presence in many types of cancer tumors, e.g. breast and colon cancer, lymphoma, leukemia, and melanoma, survivin could serve as a universal target antigen for anticancer immunotherapy.

The present invention addresses the above-mentioned need by providing recombinant nucleic acids encoding fusion proteins comprising a survivin antigen, recombinant Listeria strains comprising the same, and methods of use thereof for the treatment and prophylaxis of survivin-expressing cancers.

### SUMMARY OF THE INVENTION

In one aspect, disclosed herein is a recombinant nucleic acid molecule comprising an open reading frame encoding a recombinant polypeptide, said recombinant polypeptide comprising a heterologous antigen fused to an N-terminal Listeriolysin O (LLO) polypeptide, wherein said heterologous antigen is survivin.

In a related aspect, disclosed herein is a recombinant nucleic acid molecule comprising an open reading frame encoding a recombinant polypeptide, said recombinant polypeptide comprising a heterologous antigen fused to an N-terminal Listeriolysin O (LLO) polypeptide, wherein said heterologous antigen is survivin, wherein said nucleic acid further comprises a gram-negative origin of replication sequence operably linked to a first promoter sequence, a gram-positive origin of replication sequence, and an open reading frame encoding a metabolic enzyme operably linked to a second promoter sequence.

In another related aspect, disclosed herein is a recombinant Listeria strain comprising a recombinant nucleic acid molecule disclosed herein.

In one aspect, provided herein is a method of inducing an immune response to an antigen in a subject comprising administering a recombinant Listeria strain comprising a recombinant nucleic acid molecule, said nucleic acid molecule comprising an open reading frame encoding a polypeptide, said polypeptide comprising a heterologous antigen fused to an N-terminal Listeriolysin O (LLO) polypeptide, a N-terminal ActA polypeptide, or a PEST-peptide, wherein said heterologous antigen is survivin.

In a related aspect, provided herein is a method of treating, suppressing, or inhibiting a cancer in a subject comprising administering a recombinant Listeria strain comprising a recombinant nucleic acid molecule, said nucleic acid molecule comprising an open reading frame encoding a polypeptide, said polypeptide comprising a heterologous antigen fused to an N-terminal Listeriolysin O (LLO) polypeptide, a N-terminal ActA polypeptide, or a PEST-peptide, wherein said heterologous antigen is survivin.

In another related aspect, provided herein is a method of treating, suppressing, or inhibiting at least one tumor in a subject comprising administering a recombinant Listeria strain comprising a recombinant nucleic acid molecule, said nucleic acid molecule comprising an open reading frame encoding a polypeptide, said polypeptide comprising a heterologous antigen fused to an N-terminal Listeriolysin O (LLO) polypeptide, a N-terminal ActA polypeptide, or a PEST-peptide, wherein said heterologous antigen is survivin.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1. Shows (A) schematic representation of the chromosomal region of the Lmdd-143 and LmddA-143 after klk3 integration and actA deletion; (B) The klk3 gene is integrated into the Lmdd and LmddA chromosome. PCR from chromosomal DNA preparation from each construct using klk3 specific primers amplifies a band of 714 bp corresponding to the klk3 gene, lacking the secretion signal sequence of the wild type protein.

FIG. 2. Shows (A) map of the pADV134 plasmid. (B) Proteins from LmddA-134 culture supernatant were precipitated, separated in a SDS-PAGE, and the LLO-E7 protein detected by Western-blot using an anti-E7 monoclonal antibody. The antigen expression cassette consists of hly promoter, ORF for truncated LLO and human PSA gene (klk3). (C) Map of the pADV142 plasmid. (D) Western blot showed the expression of LLO-PSA fusion protein using anti-PSA and anti-LLO antibody.

FIG. 3. Shows (A) plasmid stability in vitro of LmddA-LLO-PSA if cultured with and without selection pressure (D-alanine). Strain and culture conditions are listed first and plates used for CFU determination are listed after. (B) Clearance of LmddA-LLO-PSA in vivo and assessment of potential plasmid loss during this time. Bacteria were injected i.v. and isolated from spleen at the time point indicated. CFUs were determined on BHI and BHI +D-alanine plates.

FIG. 4. Shows (A) In vivo clearance of the strain LmddA-LLO-PSA after administration of 10⁸ CFU in C57BL/6 mice. The number of CFU were determined by plating on BHI/str plates. The limit of detection of this method was 100 CFU. (B) Cell infection assay of J774 cells with 10403S, LmddA-LLO-PSA and XFL7 strains.

FIG. 5. Shows (A) PSA tetramer-specific cells in the splenocytes of naive and LmddA-LLO-PSA immunized mice on day 6 after the booster dose. (B) Intracellular cytokine staining for IFN-γ in the splenocytes of naive and LmddA-LLO-PSA immunized mice were stimulated with PSA peptide for 5 h. Specific lysis of EL4 cells pulsed with PSA peptide with in vitro stimulated effector T cells from LmddA-LLO-PSA immunized mice and naive mice at different effector/target ratio using a caspase based assay (C) and a europium based assay (D). Number of IFNγ spots in naive and immunized splenocytes obtained after stimulation for 24 h in the presence of PSA peptide or no peptide (E).

FIG. 6. Shows immunization with LmddA-142 induces regression of Tramp-C1-PSA (TPSA) tumors. Mice were left untreated (n=8) (A) or immunized i.p. with LmddA-142 (1×10⁸ CFU/mouse) (n=8) (B) or Lm-LLO-PSA (n=8) (C) on days 7, 14 and 21. Tumor sizes were measured for each individual tumor and the values expressed as the mean diameter in millimeters. Each line represents an individual mouse.

FIG. 7. Shows (A) Analysis of PSA-tetramer⁺CD8⁺ T cells in the spleens and infiltrating T-PSA-23 tumors of untreated mice and mice immunized with either an Lm control strain or LmddA-LLO-PSA (LmddA-142). (B) Analysis of CD4⁺ regulatory T cells, which were defined as CD25⁺FoxP3⁺, in the spleens and infiltrating T-PSA-23 tumors of untreated mice and mice immunized with either an Lm control strain or LmddA-LLO-PSA.

FIG. 8. Shows (A) Schematic representation of the chromosomal region of the Lmdd-143 and LmddA-143 after klk3 integration and actA deletion; (B) The klk3 gene is integrated into the Lmdd and LmddA chromosome. PCR from chromosomal DNA preparation from each construct using klk3 specific primers amplifies a band of 760 bp corresponding to the klk3 gene.

FIG. 9. Shows (A) Lmdd-143 and LmddA-143 secretes the LLO-PSA protein. Proteins from bacterial culture supernatants were precipitated, separated in a SDS-PAGE and LLO and LLO-PSA proteins detected by Western-blot using an anti-LLO and anti-PSA antibodies; (B) LLO produced by Lmdd-143 and LmddA-143 retains hemolytic activity. Sheep red blood cells were incubated with serial dilutions of bacterial culture supernatants and hemolytic activity measured by absorbance at 590 nm; (C) Lmdd-143 and LmddA-143 grow inside the macrophage-like J774 cells. J774 cells were incubated with bacteria for 1 hour followed by gentamicin treatment to kill extracellular bacteria. Intracellular growth was measured by plating serial dilutions of J774 lysates obtained at the indicated timepoints. Lm 10403S was used as a control in these experiments.

FIG. 10. Shows immunization of mice with Lmdd-143 and LmddA-143 induces a PSA-specific immune response. C57BL/6 mice were immunized twice at 1-week interval with 1×10⁸ CFU of Lmdd-143, LmddA-143 or LmddA-142 and 7 days later spleens were harvested. Splenocytes were stimulated for 5 hours in the presence of monensin with 1 μM of the PSA₆₅₋₇₄ peptide. Cells were stained for CD8, CD3, CD62L and intracellular IFN-γ and analyzed in a FACS Calibur cytometer.

FIG. 11. Shows three Lm-based vaccines expressing distinct HMW-MAA fragments based on the position of previously mapped and predicted HLA-A2 epitopes were designed (A). The Lm-tLLO-HMW-MMA₂₁₆₀₋₂₂₅₈ (also referred as Lm-LLO-HMW-MAA-C) strain secretes a ˜62 kDa band corresponding to the tLLO-HMW-MAA₂₁₆₀₋₂₂₅₈ fusion protein (B). C57BL/6 mice (n=15) were inoculated s.c. with B16F10 cells and either immunized i.p. on days 3, 10 and 17 with Lm-tLLO-HMW-MAA₂₁₆₀₋₂₂₅₈ (n=8) or left untreated (n=7). BALB/c mice (n=16) were inoculated s.c. with RENCA cells and immunized i.p. on days 3, 10 and 17 with either Lm-HMW-MAA-C (n=8) or an equivalent dose of a control Lm vaccine. Mice immunized with the Lm-LLO-HMW-MAA-C impeded the growth of established tumors (C). FVB/N mice (n=13) were inoculated s.c. with NT-2 tumor cells and immunized i.p. on days 7, 14 and 21 with either Lm-HMW-MAA-C (n=5) or an equivalent dose of a control Lm vaccine (n=8) Immunization of mice with Lm-LLO-HMW-MAA-C significantly impaired the growth of tumors not engineered to express HMW-MAA, such as B16F10, RENCA and NT-2 (D). Tumor sizes were measured for each individual tumor and the values expressed as the mean diameter in millimeters±SEM. *, P≦0.05, Mann-Whitney test.

FIG. 12. Shows that immunization with Lm-HMW-MAA-C promotes tumor infiltration by CD8⁺ T cells and decreases the number of pericytes in blood vessels. (A) NT-2 tumors were removed and sectioned for immunofluorescence. Staining groups are numbered (1-3) and each stain is indicated on the right. Sequential tissues were either stained with the pan-vessel marker anti-CD31 or the anti-NG2 antibody for the HMW-MAA mouse homolog AN2, in conjunction with anti-CD8α for possible TILs. Group 3 shows isotype controls for the above antibodies and DAPI staining used as a nuclear marker. A total of 5 tumors were analyzed and a single representative image from each group is shown. CD8⁺ cells around blood vessels are indicated by arrows. (B) Sequential sections were stained for pericytes by using the anti-NG2 and anti-alpha-smooth-muscle-cell-actin (α-SMA) antibodies. Double staining/colocalization of these two antibodies (yellow in merge image) are indicative of pericyte staining (top). Pericyte colocalization was quantitated using Image Pro Software and the number of colocalized objects is shown in the graph (bottom). A total of 3 tumors were analyzed and a single representative image from each group is shown. *, P≦0.05, Mann-Whitney test. Graph shows mean±SEM.

FIG. 13. Shows a gel showing the size of PCR products using oligos 554/555 for mouse survivin and oligos 552/553 for human survivin fragment obtained using m-RNA sequences of the strains as template.

FIG. 14. Shows schematic maps of the plasmids pAdv266.7 (A) and pAdv265.5 (B). The plasmids contain both Listeria and E. coli origin of replication. The antigen expression cassette consists of hly promoter, ORF for truncated LLO and human or mouse survivin gene.

FIG. 15. Shows western blots from LmddA-LLO-survivin supernatants shows the expression of chromosomal LLO protein detected using the monoclonal antibody anti-B3-19, truncated LLO-Survivin fusion protein and disintegrated t-LLO protein detected using polyclonal antibody anti-PEST and as well as tLLO-Survivin fusion protein detected using the monoclonal antibody anti-survivin antibody.

FIG. 16. Shows the western blot from LmddA-LLO-survivin supernatants shows the expression and secretion of tLLO-Survivin fusion protein after second in vivo passage using anti-survivin antibody.

FIG. 17. Shows the reduction of NT-2 tumor growth after treatment with Listeria-based immunotherapy expressing survivin.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In one embodiment, disclosed herein is a recombinant nucleic acid molecule comprising an open reading frame encoding a recombinant polypeptide, said recombinant polypeptide comprising a heterologous antigen fused to an N-terminal Listeriolysin O (LLO) polypeptide, wherein said heterologous antigen is survivin.

In another embodiment, the recombinant nucleic acid molecule disclosed herein is a DNA vector, wherein in another embodiment it is a plasmid.

In another embodiment, the gram-negative origin of replication sequence disclosed herein is any gram-negative origin of replication (Ori) available in the art. In another embodiment, the gram-negative Ori is an E. coli Ori. In another embodiment, the gram-negative Ori is a p15 sequence.

In another embodiment, the gram-positive origin of replication sequence disclosed herein is any gram-negative origin of replication (Ori) available in the art. In another embodiment, the gram-negative Ori is a Rep R sequence or region.

In another embodiment, “truncated LLO” or “ΔLLO” refers to a fragment of LLO that comprises a putative PEST amino acid sequence. In another embodiment, the terms refer to an LLO fragment that comprises a putative PEST domain. In another embodiment, ther terms “truncated LLO” and “N-terminal LLO” are used interchangeably herein.

In another embodiment, disclosed herein is a recombinant nucleic acid molecule comprising an open reading frame encoding a recombinant polypeptide, said recombinant polypeptide comprising a heterologous antigen fused to an N-terminal Listeriolysin O (LLO) polypeptide, wherein said heterologous antigen is survivin, wherein said nucleic acid further comprises a gram-negative origin of replication sequence operably linked to a first promoter sequence, a gram-positive origin of replication sequence, and an open reading frame encoding a metabolic enzyme operably linked to a second promoter sequence.

In another embodiment, disclosed herein is a recombinant Listeria strain comprising a recombinant nucleic acid molecule disclosed herein.

This invention relates, in one embodiment, to a recombinant Listeria strain comprising a recombinant nucleic acid molecule, said nucleic acid molecule comprising an open reading frame encoding a polypeptide, said polypeptide comprising a heterologous antigen fused to an N-terminal Listeriolysin O (LLO) polypeptide, a N-terminal ActA polypeptide, or a PEST-peptide, and wherein said heterologous antigen is survivin.

In another embodiment, provided herein is a method of inducing an immune response to an antigen in a subject comprising administering a recombinant Listeria strain comprising a recombinant nucleic acid molecule, said nucleic acid molecule comprising an open reading frame encoding a polypeptide, said polypeptide comprising a heterologous antigen fused to an N-terminal Listeriolysin O (LLO) polypeptide, a N-terminal ActA polypeptide, or a PEST-peptide, and wherein said heterologous antigen is survivin.

In another embodiment, provided herein is a method of treating, suppressing, or inhibiting a cancer in a subject comprising administering a recombinant Listeria strain comprising a recombinant nucleic acid molecule, said nucleic acid molecule comprising an open reading frame encoding a polypeptide, said polypeptide comprising a heterologous antigen fused to an N-terminal Listeriolysin O (LLO) polypeptide, a N-terminal ActA polypeptide, or a PEST-peptide, and wherein said heterologous antigen is survivin.

In another embodiment, provided herein is a method of treating, suppressing, or inhibiting at least one tumor in a subject comprising administering a recombinant Listeria strain comprising a recombinant nucleic acid molecule, said nucleic acid molecule comprising an open reading frame encoding a polypeptide, said polypeptide comprising a heterologous antigen fused to an N-terminal Listeriolysin O (LLO) polypeptide, a N-terminal ActA polypeptide, or a PEST-peptide, and wherein said heterologous antigen is survivin.

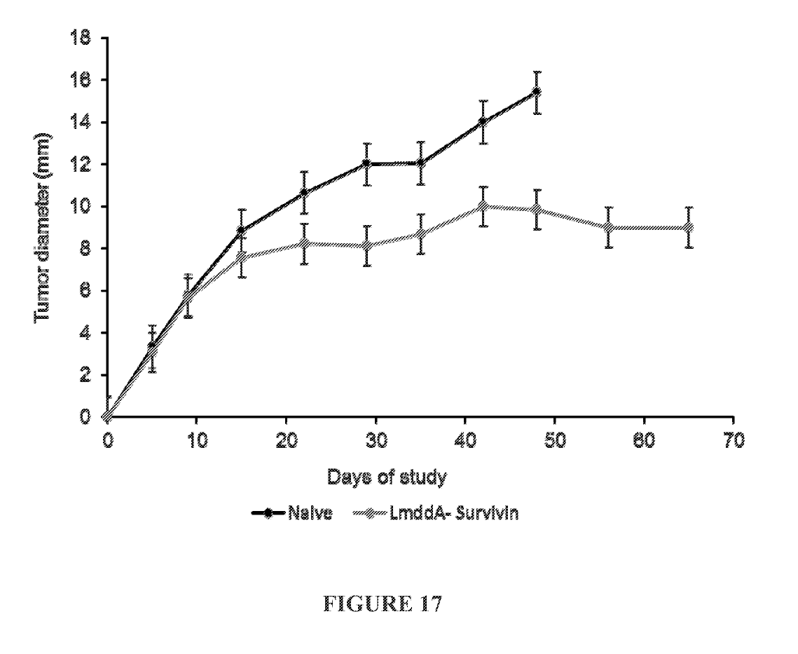

In one embodiment, said heterologous antigen is survivin. As demonstrated herein, a recombinant Listeria comprising a recombinant nucleic acid encoding a tLLO-survivin fusion protein can reduce tumor growth and for an unexpectedly prolonged period as compared to control (see Example 16 and FIG. 17 herein).

In one embodiment, a N-terminal Listeriolysin O (LLO) polypeptide, and a N-terminal ActA polypeptide comprise a PEST sequence.

In one embodiment, the nucleic acid molecule is operably integrated into the Listeria genome as an open reading frame with an endogenous nucleic acid sequence encoding a polypeptide comprising a PEST sequence. In one embodiment, the nucleic acid molecule is operably integrated into the Listeria genome as an open reading frame with a nucleic acid sequence encoding LLO. In another embodiment, the nucleic acid molecule is operably integrated into the Listeria genome as an open reading frame with a nucleic acid sequence encoding ActA.

In one embodiment, the nucleic acid molecule is present in a plasmid in said recombinant Listeria.

In one embodiment, the nucleic acid molecule is operably integrated into the Listeria genome in an open reading frame with an endogenous nucleic acid sequence encoding LLO. In one embodiment, the integration does not eliminate the functionality of LLO. In another embodiment, the integration does not eliminate the functionality of ActA. In one embodiment, the functionality of LLO or ActA is its native functionality. In one embodiment, the LLO functionality is allowing the organism to escape from the phagolysosome, while in another embodiment, the LLO functionality is enhancing the immunogenicity of a polypeptide to which it is fused.

In one embodiment, the nucleic acid molecule is operably integrated into a virulence gene in the Listeria genome. In another embodiment, the virulence gene comprises an actA gene, an internalin gene such as inlA, inlB, or inlC, a prfA gene, or an LLO gene. In another embodiment, the integration into the virulence gene disrupts the native function of the virulence gene. In another embodiment, the integration inactivates the virulence gene. In another embodiment, the integration into the virulence gene does not disrupt the native function of the virulence gene. In one embodiment, a recombinant Listeria of the present invention retains LLO function, which in one embodiment, is hemolytic function and in another embodiment, is antigenic function. Other functions of LLO are known in the art, as are methods of and assays for evaluating LLO functionality. In one embodiment, a recombinant Listeria of the present invention has wild-type virulence, while in another embodiment, a recombinant Listeria of the present invention has attenuated virulence. In another embodiment, a recombinant Listeria of the present invention is avirulent. In one embodiment, a recombinant Listeria of the present invention is sufficiently virulent to escape the phagolysosome and enter the cytosol. In one embodiment, a recombinant Listeria of the present invention expresses a fused antigen-LLO protein. Thus, in one embodiment, the integration of the first nucleic acid molecule into the Listeria genome does not disrupt the structure of the endogenous PEST-containing gene, while in another embodiment, it does not disrupt the function of the endogenous PEST-containing gene. In one embodiment, the integration of the first nucleic acid molecule into the Listeria genome does not disrupt the ability of said Listeria to escape the phagolysosome.

In another embodiment, the nucleic acid molecule is present in a plasmid in said recombinant Listeria and comprises an open reading frame encoding a heterologous antigen operably linked to an endogenoues PEST-containing polypeptide or PEST sequence. In one embodiment, the heterologous antigenic polypeptide and the endogenous PEST-containing polypeptide are translated in a single open reading frame, while in another embodiment, the heterologous antigenic polypeptide and the endogenous PEST-containing polypeptide are fused after being translated separately.

In one embodiment, the Listeria genome comprises a deletion of the endogenous ActA gene, which in one embodiment is a virulence factor. In one embodiment, such a deletion provides a more attenuated and thus safer Listeria strain for human use. In one embodiment, the Listeria is auxotrophic for the dal/dat genes. In another embodiment, the dal/dat genes are mutated in the Listeria genome. In another embodiment, the recombinant Listeria strain is an auxotrophic dal/dat mutant. In another embodiment, the recombinant Listeria strain is an auxotrophic dal/dat mutant Listeria lacking an endogenous actA gene.

In one embodiment, the heterologous antigen is integrated in frame with LLO in the Listeria chromosome. In another embodiment, the integrated nucleic acid molecule is integrated into the ActA locus. In another embodiment, the chromosomal nucleic acid encoding ActA is replaced by a nucleic acid molecule encoding an antigen.

In one embodiment, the nucleic acid molecule is a vector designed for site-specific homologous recombination into the Listeria genome. In another embodiment, the construct or heterologous gene is integrated into the Listerial chromosome using homologous recombination.

Techniques for homologous recombination are well known in the art, and are described, for example, in Frankel, F R, Hegde, S, Lieberman, J, and Y Paterson. Induction of a cell-mediated immune response to HIV gag using Listeria monocytogenes as a live vaccine vector. J. Immunol. 155: 4766-4774. 1995; Mata, M, Yao, Z, Zubair, A, Syres, K and Y Paterson, Evaluation of a recombinant Listeria monocytogenes expressing an HIV protein that protects mice against viral challenge. Vaccine 19:1435-45, 2001; Boyer, J D, Robinson, T M, Maciag, P C, Peng, X, Johnson, R S, Pavlakis, G, Lewis, M G, Shen, A, Siliciano, R, Brown, C R, Weiner, D, and Y Paterson. DNA prime Listeria boost induces a cellular immune response to SIV antigens in the Rhesus Macaque model that is capable of limited suppression of SIV239 viral replication. Virology. 333: 88-101, 2005. In another embodiment, homologous recombination is performed as described in U.S. Pat. No. 6,855,320. In another embodiment, a temperature sensitive plasmid is used to select the recombinants. Each technique represents a separate embodiment of the methods and compositions disclosed herein.

In another embodiment, the construct or heterologous gene is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in one embodiment, that a stable genomic insertion mutant can be formed. In another embodiment, the position in the genome where the foreign gene has been inserted by transposon mutagenesis is unknown.

In another embodiment, the construct or heterologous gene is integrated into the Listerial chromosome using phage integration sites (Lauer P, Chow M Y et al, Construction, characterization, and use of two LM site-specific phage integration vectors. J Bacteriol 2002;184(15): 4177-86). In another embodiment, an integrase gene and attachment site of a bacteriophage (e.g. U153 or PSA listeriophage) is used to insert the heterologous gene into the corresponding attachment site, which can be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. Each possibility represents a separate embodiment disclosed herein.

In another embodiment, the nucleic acid sequence of methods and compositions disclosed herein is operably linked to a promoter/regulatory sequence. In one embodiment, the promoter/regulatory sequence is present on an episomal plasmid comprising said nucleic acid sequence. In one embodiment, endogenous Listeria promoter/regulatory sequence controls the expression of a nucleic acid sequence of the methods and compositions of the present invention. Each possibility represents a separate embodiment of the methods and compositions disclosed herein.

In another embodiment, a nucleic acid sequence disclosed herein is operably linked to a promoter, regulatory sequence, or combination thereof that drives expression of the encoded peptide in the Listeria strain. Promoter, regulatory sequences, and combinations thereof useful for driving constitutive expression of a gene are well known in the art and include, but are not limited to, for example, the P_{hlyA}, P_{ActA}, hly, actA, and p60 promoters of Listeria, the Streptococcus bac promoter, the Streptomyces griseus sgiA promoter, and the B. thuringiensis phaZ promoter. In another embodiment, inducible and tissue specific expression of the nucleic acid encoding a peptide disclosed herein is accomplished by placing the nucleic acid encoding the peptide under the control of an inducible or tissue-specific promoter/regulatory sequence. Examples of tissue-specific or inducible regulatory sequences, promoters, and combinations thereof which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter or regulatory sequence, which is either known or unknown, and which is capable of driving expression of the desired protein operably linked thereto. In one embodiment, a regulatory sequence is a promoter, while in another embodiment, a regulatory sequence is an enhancer, while in another embodiment, a regulatory sequence is a suppressor, while in another embodiment, a regulatory sequence is a repressor, while in another embodiment, a regulatory sequence is a silencer.

It will be appreciated by a skilled artisan that a nucleic acid construct used for integration comprises an integration site. In one embodiment, when used for integration into the Listeria genome, the site is a PhSA (phage from Scott A) attPP′ integration site. PhSA is, in another embodiment, the prophage of L. monocytogenes strain ScottA (Loessner, M. J., I. B. Krause, T. Henle, and S. Scherer. 1994. Structural proteins and DNA characteristics of 14 Listeria typing bacteriophages. J. Gen. Virol. 75:701-710, incorporated herein by reference), a serotype 4b strain that was isolated during an epidemic of human listeriosis. In another embodiment, the site is any another integration site known in the art. Each possibility represents a separate embodiment of the methods and compositions disclosed herein.

In another embodiment, the nucleic acid construct contains an integrase gene. In another embodiment, the integrase gene is a PhSA integrase gene. In another embodiment, the integrase gene is any other integrase gene known in the art. Each possibility represents a separate embodiment of the methods and compositions disclosed herein.

In one embodiment, the nucleic acid construct is a plasmid. In another embodiment, the nucleic acid construct is a shuttle plasmid. In another embodiment, the nucleic acid construct is an integration vector. In another embodiment, the nucleic acid construct is a site-specific integration vector. In another embodiment, the nucleic acid construct is any other type of nucleic acid construct known in the art. Each possibility represents a separate embodiment of the methods and compositions provided herein.

The integration vector of the methods and compositions provided herein is, in another embodiment, a phage vector. In another embodiment, the integration vector is a site-specific integration vector. In another embodiment, the vector further comprises an attPP′ site. Each possibility represents a separate embodiment of the methods and compositions disclosed herein.

In another embodiment, the integration vector is a U153 vector. In another embodiment, the integration vector is an A118 vector. In another embodiment, the integration vector is a PhSA vector.

In another embodiment, the vector is an A511 vector (e.g. GenBank Accession No: X91069). In another embodiment, the vector is an A006 vector. In another embodiment, the vector is a B545 vector. In another embodiment, the vector is a B053 vector. In another embodiment, the vector is an A020 vector. In another embodiment, the vector is an A500 vector (e.g. GenBank Accession No: X85009). In another embodiment, the vector is a B051 vector. In another embodiment, the vector is a B052 vector. In another embodiment, the vector is a B054 vector. In another embodiment, the vector is a B055 vector. In another embodiment, the vector is a B056 vector. In another embodiment, the vector is a B101 vector. In another embodiment, the vector is a B110 vector. In another embodiment, the vector is a B111 vector. In another embodiment, the vector is an A153 vector. In another embodiment, the vector is a D441 vector. In another embodiment, the vector is an A538 vector. In another embodiment, the vector is a B653 vector. In another embodiment, the vector is an A513 vector. In another embodiment, the vector is an A507 vector. In another embodiment, the vector is an A502 vector. In another embodiment, the vector is an A505 vector. In another embodiment, the vector is an A519 vector. In another embodiment, the vector is a B604 vector. In another embodiment, the vector is a C703 vector. In another embodiment, the vector is a B025 vector. In another embodiment, the vector is an A528 vector. In another embodiment, the vector is a B024 vector. In another embodiment, the vector is a B012 vector. In another embodiment, the vector is a B035 vector. In another embodiment, the vector is a C707 vector.

In another embodiment, the vector is an A005 vector. In another embodiment, the vector is an A620 vector. In another embodiment, the vector is an A640 vector. In another embodiment, the vector is a B021 vector. In another embodiment, the vector is an HS047 vector. In another embodiment, the vector is an H10G vector. In another embodiment, the vector is an H8/73 vector. In another embodiment, the vector is an H19 vector. In another embodiment, the vector is an H21 vector. In another embodiment, the vector is an H43 vector. In another embodiment, the vector is an H46 vector. In another embodiment, the vector is an H107 vector. In another embodiment, the vector is an H108 vector. In another embodiment, the vector is an H110 vector. In another embodiment, the vector is an H163/84 vector. In another embodiment, the vector is an H312 vector. In another embodiment, the vector is an H340 vector. In another embodiment, the vector is an H387 vector. In another embodiment, the vector is an H391/73 vector. In another embodiment, the vector is an H684/74 vector. In another embodiment, the vector is an H924A vector. In another embodiment, the vector is an fMLUP5 vector. In another embodiment, the vector is a syn(=P35) vector. In another embodiment, the vector is a 00241 vector. In another embodiment, the vector is a 00611 vector. In another embodiment, the vector is a 02971A vector. In another embodiment, the vector is a 02971C vector. In another embodiment, the vector is a 5/476 vector. In another embodiment, the vector is a 5/911 vector. In another embodiment, the vector is a 5/939 vector. In another embodiment, the vector is a 5/11302 vector. In another embodiment, the vector is a 5/11605 vector. In another embodiment, the vector is a 5/11704 vector. In another embodiment, the vector is a 184 vector. In another embodiment, the vector is a 575 vector. In another embodiment, the vector is a 633 vector. In another embodiment, the vector is a 699/694 vector. In another embodiment, the vector is a 744 vector. In another embodiment, the vector is a 900 vector. In another embodiment, the vector is a 1090 vector. In another embodiment, the vector is a 1317 vector. In another embodiment, the vector is a 1444 vector. In another embodiment, the vector is a 1652 vector. In another embodiment, the vector is a 1806 vector. In another embodiment, the vector is a 1807 vector. In another embodiment, the vector is a 1921/959 vector. In another embodiment, the vector is a 1921/11367 vector. In another embodiment, the vector is a 1921/11500 vector. In another embodiment, the vector is a 1921/11566 vector. In another embodiment, the vector is a 1921/12460 vector. In another embodiment, the vector is a 1921/12582 vector. In another embodiment, the vector is a 1967 vector. In another embodiment, the vector is a 2389 vector. In another embodiment, the vector is a 2425 vector. In another embodiment, the vector is a 2671 vector. In another embodiment, the vector is a 2685 vector. In another embodiment, the vector is a 3274 vector. In another embodiment, the vector is a 3550 vector. In another embodiment, the vector is a 3551 vector. In another embodiment, the vector is a 3552 vector. In another embodiment, the vector is a 4276 vector. In another embodiment, the vector is a 4277 vector. In another embodiment, the vector is a 4292 vector. In another embodiment, the vector is a 4477 vector. In another embodiment, the vector is a 5337 vector. In another embodiment, the vector is a 5348/11363 vector. In another embodiment, the vector is a 5348/11646 vector. In another embodiment, the vector is a 5348/12430 vector. In another embodiment, the vector is a 5348/12434 vector. In another embodiment, the vector is a 10072 vector. In another embodiment, the vector is a 11355C vector. In another embodiment, the vector is a 11711A vector. In another embodiment, the vector is a 12029 vector. In another embodiment, the vector is a 12981 vector. In another embodiment, the vector is a 13441 vector. In another embodiment, the vector is a 90666 vector. In another embodiment, the vector is a 90816 vector. In another embodiment, the vector is a 93253 vector. In another embodiment, the vector is a 907515 vector. In another embodiment, the vector is a 910716 vector. In another embodiment, the vector is a NN-Listeria vector. In another embodiment, the vector is a 01761 vector. In another embodiment, the vector is a 4211 vector. In another embodiment, the vector is a 4286 vector.

In another embodiment, the integration vector is any other site-specific integration vector known in the art that is capable of infecting Listeria. Each possibility represents a separate embodiment of the methods and compositions disclosed herein. In another embodiment, the integration vector or plasmid of methods and compositions disclosed herein does not confer antibiotic resistance to the Listeria vaccine strain. In another embodiment, the integration vector or plasmid does not contain an antibiotic resistance gene.

In another embodiment, the present invention provides an isolated nucleic acid encoding a recombinant polypeptide. In one embodiment, the isolated nucleic acid comprises a sequence sharing at least 70% homology with a nucleic acid encoding a recombinant polypeptide provided herein. In another embodiment, the isolated nucleic acid comprises a sequence sharing at least 75% homology with a nucleic acid encoding a recombinant polypeptide provided herein. In another embodiment, the isolated nucleic acid comprises a sequence sharing at least 80% homology with a nucleic acid encoding a recombinant polypeptide provided herein. In another embodiment, the isolated nucleic acid comprises a sequence sharing at least 85% homology with a nucleic acid encoding a recombinant polypeptide provided herein. In another embodiment, the isolated nucleic acid comprises a sequence sharing at least 90% homology with a nucleic acid encoding a recombinant polypeptide provided herein. In another embodiment, the isolated nucleic acid comprises a sequence sharing at least 95% homology with a nucleic acid encoding a recombinant polypeptide provided herein. In another embodiment, the isolated nucleic acid comprises a sequence sharing at least 97% homology with a nucleic acid encoding a recombinant polypeptideprovided herein. In another embodiment, the isolated nucleic acid comprises a sequence sharing at least 99% homology with a nucleic acid encoding a recombinant polypeptide provided herein.

In one embodiment, provided herien is a method of producing a recombinant Listeria expressing a heterologous antigen provided herein. In another embodiment, the method comprises transforming said recombinant Listeria with an episomal expression vector comprising a nucleic acid encoding said heterologous antigen. In another embodiment, the method comprises expressing said antigen under conditions conducive to antigenic expression, that are known in the art, in said recombinant Listeria strain.

In one embodiment, the antigen is expressed as a fusion protein with LLO, which in one embodiment, is non-hemolytic LLO, and in another embodiment, is a truncated LLO. In another embodiment, the antigen is expressed as a fusion protein with a N-terminal ActA protein, which in one embodiment, is a truncated ActA.

In another embodiment, a recombinant Listeria strain provided herein targets tumors by eliciting immune responses to the antigen expressed thereby.

In another embodiment, an episomal expression vector of the methods and compositions provided herein comprises an antigen fused in frame to a nucleic acid sequence encoding a PEST amino acid (AA) sequence. In one embodiment, the antigen is survivin. In another embodiment, the antigen is a survivin fragment. In another embodiment, the antigen is an immunogenic fragment of a survivin fragment. In another embodiment, the PEST AA sequence is KENSISSMAPPASPPASPKTPIEKKHADEIDK (SEQ ID NO: 1). In another embodiment, the PEST sequence is KENSISSMAPPASPPASPK (SEQ ID No: 2). In another embodiment, fusion of an antigen to any LLO sequence that includes one of the PEST AA sequences enumerated herein can enhance cell mediated immunity against survivin.

In another embodiment, the PEST AA sequence is a PEST sequence from a Listeria ActA protein. In another embodiment, the PEST sequence is KTEEQPSEVNTGPR (SEQ ID NO: 3), KASVTDTSEGDLDSSMQSADESTPQPLK (SEQ ID NO: 4), KNEEVNASDFPPPPTDEELR (SEQ ID NO: 5), or RGGIPTSEEFSSLNSGDFTDDENSETTEEEIDR (SEQ ID NO: 6). In another embodiment, the PEST sequence is from Listeria seeligeri cytolysin, encoded by the lso gene. In another embodiment, the PEST sequence is RSEVTISPAETPESPPATP (SEQ ID NO: 7). In another embodiment, the PEST sequence is from Streptolysin O protein of Streptococcus sp. In another embodiment, the PEST sequence is from Streptococcus pyogenes Streptolysin O, e.g. KQNTASTETTTTNEQPK (SEQ ID NO: 8) at AA 35-51. In another embodiment, the PEST sequence is from Streptococcus equisimilis Streptolysin O, e.g. KQNTANTETTTTNEQPK (SEQ ID NO: 9) at AA 38-54. In another embodiment, the PEST sequence has a sequence selected from SEQ ID NO: 3-9. In another embodiment, the PEST sequence has a sequence selected from SEQ ID NO: 1-9. In another embodiment, the PEST sequence is another PEST AA sequence derived from a prokaryotic organism.

Identification of PEST sequences is well known in the art, and is described, for example in Rogers S et al (Amino acid sequences common to rapidly degraded proteins: the PEST hypothesis. Science 1986; 234(4774):364-8, incorporated herein by reference) and Rechsteiner M et al (PEST sequences and regulation by proteolysis. Trends Biochem Sci 1996; 21(7):267-71, incorporated herein by reference). “PEST sequence” refers, in another embodiment, to a region rich in proline (P), glutamic acid (E), serine (S), and threonine (T) residues. In another embodiment, the PEST sequence is flanked by one or more clusters containing several positively charged amino acids. In another embodiment, the PEST sequence mediates rapid intracellular degradation of proteins containing it. In another embodiment, the PEST sequence fits an algorithm disclosed in Rogers et al. In another embodiment, the PEST sequence fits an algorithm disclosed in Rechsteiner et al. In another embodiment, the PEST sequence contains one or more internal phosphorylation sites, and phosphorylation at these sites precedes protein degradation. In one embodiment, a sequence referred to herein as a PEST sequence is a PEST sequence. In another embodiment a PEST sequence is a PEST petide sequence or simply a PEST peptide.

In one embodiment, PEST sequences of prokaryotic organisms are identified in accordance with methods such as described by, for example Rechsteiner and Rogers (1996, Trends Biochem. Sci. 21:267-271) for LM and in Rogers S et al (Science 1986; 234(4774):364-8). Alternatively, PEST AA sequences from other prokaryotic organisms can also be identified based on this method. Other prokaryotic organisms wherein PEST AA sequences would be expected to include, but are not limited to, other Listeria species. In one embodiment, the PEST sequence fits an algorithm disclosed in Rogers et al. In another embodiment, the PEST sequence fits an algorithm disclosed in Rechsteiner et al. In another embodiment, the PEST sequence is identified using the PEST-find program.

In another embodiment, identification of PEST motifs is achieved by an initial scan for positively charged amino acids R, H, and K within the specified protein sequence. All amino acids between the positively charged flanks are counted and only those motifs are considered further, which contain a number of amino acids equal to or higher than the window-size parameter. In another embodiment, a PEST sequence must contain at least 1 P, 1 D or E, and at least 1 S or T.

In another embodiment, the quality of a PEST motif is refined by means of a scoring parameter based on the local enrichment of critical amino acids as well as the motifs hydrophobicity. Enrichment of D, E, P, S and T is expressed in mass percent (w/w) and corrected for 1 equivalent of D or E, 1 of P and 1 of S or T. In another embodiment, calculation of hydrophobicity follows in principle the method of J. Kyte and R. F. Doolittle (Kyte, J and Dootlittle, R F. J. Mol. Biol. 157, 105 (1982), incorporated herein by reference. For simplified calculations, Kyte-Doolittle hydropathy indices, which originally ranged from −4.5 for arginine to +4.5 for isoleucine, are converted to positive integers, using the following linear transformation, which yielded values from 0 for arginine to 90 for isoleucine.

Hydropathy index=10*Kyte-Doolittle hydropathy index+45

In another embodiment, a potential PEST motif's hydrophobicity is calculated as the sum over the products of mole percent and hydrophobicity index for each amino acid species. The desired PEST score is obtained as combination of local enrichment term and hydrophobicity term as expressed by the following equation:

PEST score=0.55*DEPST−0.5*hydrophobicity index.

In another embodiment, the terms “PEST sequence,” “PEST sequence” or “PEST peptide” are used interchangeably and refer to a peptide having a score of at least +5, using the above algorithm. In another embodiment, the term refers to a peptide having a score of at least 6. In another embodiment, the peptide has a score of at least 7. In another embodiment, the score is at least 8. In another embodiment, the score is at least 9. In another embodiment, the score is at least 10. In another embodiment, the score is at least 11. In another embodiment, the score is at least 12. In another embodiment, the score is at least 13. In another embodiment, the score is at least 14. In another embodiment, the score is at least 15. In another embodiment, the score is at least 16. In another embodiment, the score is at least 17. In another embodiment, the score is at least 18. In another embodiment, the score is at least 19. In another embodiment, the score is at least 20. In another embodiment, the score is at least 21. In another embodiment, the score is at least 22. In another embodiment, the score is at least 22. In another embodiment, the score is at least 24. In another embodiment, the score is at least 24. In another embodiment, the score is at least 25. In another embodiment, the score is at least 26. In another embodiment, the score is at least 27. In another embodiment, the score is at least 28. In another embodiment, the score is at least 29. In another embodiment, the score is at least 30. In another embodiment, the score is at least 32. In another embodiment, the score is at least 35. In another embodiment, the score is at least 38. In another embodiment, the score is at least 40. In another embodiment, the score is at least 45. Each possibility represents a separate embodiment of the methods and compositions disclosed herein.

In another embodiment, the PEST sequence is identified using any other method or algorithm known in the art, e.g the CaSPredictor (Garay-Malpartida H M, Occhiucci J M, Alves J, Belizario J E. Bioinformatics. 2005 Jun;21 Suppl 1:i169-76). In another embodiment, the following method is used:

A PEST index is calculated for each stretch of appropriate length (e.g. a 30-35 amino acid stretch) by assigning a value of 1 to the amino acids Ser, Thr, Pro, Glu, Asp, Asn, or Gln. The coefficient value (CV) for each of the PEST residue is 1 and for each of the other amino acids (non-PEST) is 0.

In another embodiment, the PEST sequence is any other PEST sequence known in the art.

In one embodiment, the present invention provides fusion proteins, which in one embodiment, are expressed by Listeria. In one embodiment, such fusion proteins are fused to a PEST sequence which, in one embodiment, refers to fusion to a protein fragment comprising a PEST sequence. In another embodiment, the term includes cases wherein the protein fragment comprises surrounding sequence other than the PEST sequence. In another embodiment, the protein fragment consists of the PEST sequence. Thus, in another embodiment, “fusion” refers to two peptides or protein fragments either linked together at their respective ends or embedded one within the other.

In another embodiment, an LLO protein fragment is utilized in compositions and methods disclosed herein. In another embodiment, a recombinant Listeria strain of the compositions and methods provided herein comprises a full length LLO polypeptide, which in one embodiment, is hemolytic. In another embodiment, the recombinant Listeria strain comprises a non-hemolytic LLO polypeptide. In one embodiment a hemolytic LLO polypeptide is expressed from the Listeria chromosome whereas a non-hemolytic LLO polypeptide is expressed from an episomal plasmid, present in the cytoplasm of the Listeria, in the form of a fusion protein with an antigen.

In another embodiment, the LLO polypeptide is a fragment of an LLO polypeptide. In another embodiment, the LLO polypeptide is an N-terminal LLO fragment. In another embodiment, the polypeptide is a detox LLO, as described in US Patent Publication Serial No. 2009/0081248, which is also incorporated by reference herein in its entirety. In another embodiment, the oligopeptide is a complete LLO protein. In another embodiment, the polypeptide is any LLO protein or fragment thereof known in the art. disclosed herein

In one embodiment, a truncated LLO protein is encoded by the episomal expression vector disclosed herein that expresses a polypeptide, that is, in one embodiment, an antigen, in another embodiment, an angiogenic factor, or, in another embodiment, both an antigen and angiogenic factor. In another embodiment, the LLO fragment is an N-terminal fragment.

In another embodiment, the N-terminal LLO fragment has the sequence:

MKKIMLVFITLILVSLPIAQQTEAKDASAFNKENSISSVAPPASPPASPKTPIE KKHADEIDKYIQGLDYNKNNVLVYHGDAVTNVPPRKGYKDGNEYIVVEKKKKSIN QNNADIQVVNAISSLTYPGALVKANSELVENQPDVLPVKRDSLTLSIDLPGMTNQDN KIVVKNATKSNVNNAVNTLVERWNEKYAQAYSNVSAKIDYDDEMAYSESQLIAKF GTAFKAVNNSLNVNFGAISEGKMQEEVISFKQIYYNVNVNEPTRPSRFFGKAVTKEQ LQALGVNAENPPAYISSVAYGRQVYLKLSTNSHSTKVKAAFDAAVSGKSVSGDVEL TNIIKNSSFKAVIYGGSAKDEVQIIDGNLGDLRDILKKGATFNRETPGVPIAYTTNFLK DNELAVIKNNSEYIETTSKAYTDGKINIDHSGGYVAQFNISWDEVNYD (SEQ ID NO: 10). In another embodiment, an LLO AA sequence of methods and compositions disclosed herein comprises the sequence set forth in SEQ ID No: 10. In another embodiment, the LLO AA sequence is a homologue of SEQ ID No: 10. In another embodiment, the LLO AA sequence is a variant of SEQ ID No: 10. In another embodiment, the LLO AA sequence is a fragment of SEQ ID No: 10. In another embodiment, the LLO AA sequence is an isoform of SEQ ID No: 10.

In another embodiment, the LLO fragment has the sequence:

MKKIMLVFITLILVSLPIAQQTEAKDASAFNKENSISSVAPPASPPASPKTPIE KKHADEIDKYIQGLDYNKNNVLVYIIGDAVTNVPPRKGYKDGNEYIVVEKKKKSIN QNNADIQVVNAISSLTYPGALVKANSELVENQPDVLPVKRDSLTLSIDLPGMTNQDN KIVVKNATKSNVNNAVNTLVERWNEKYAQAYSNVSAKIDYDDEMAYSESQLIAKF GTAFKAVNNSLNVNFGAISEGKMQEEVISFKQIYYNVNVNEPTRPSRFFGKAVTKEQ LQALGVNAENPPAYISSVAYGRQVYLKLSTNSHSTKVKAAFDAAVSGKSVSGDVEL TNIIKNSSFKAVIYGGSAKDEVQIIDGNLGDLRDILKKGATFNRETPGVPIAYTTNFLK DNELAVIKNNSEYIETTSKAYTD (SEQ ID NO: 11). In another embodiment, an LLO AA sequence of methods and compositions disclosed herein comprises the sequence set forth in SEQ ID No: 11. In another embodiment, the LLO AA sequence is a homologue of SEQ ID No: 11. In another embodiment, the LLO AA sequence is a variant of SEQ ID No: 11. In another embodiment, the LLO AA sequence is a fragment of SEQ ID No: 11. In another embodiment, the LLO AA sequence is an isoform of SEQ ID No: 11.

The LLO protein used in the compositions and methods disclosed herein comprises, in another embodiment, the sequence:

MKKIMLVFITLILVSLPIAQQTEAKDASAFNKENSISSMAPPASPPASPKTPIE KKHADEIDKYIQGLDYNKNNVLVYHGDAVTNVPPRKGYKDGNEYIVVEKKKKSIN QNNADIQVVNAISSLTYPGALVKANSELVENQPDVLPVKRDSLTLSIDLPGMTNQD NKIVVKNATKSNVNNAVNTLVERWNEKYAQAYPNVSAKIDYDDEMAYSESQLIAK FGTAFKAVNNSLNVNFGAISEGKMQEEVISFKQIYYNVNVNEPTRPSRFFGKAVTKE QLQALGVNAENPPAYISSVAYGRQVYLKLSTNSHSTKVKAAFDAAVSGKSVSGDV ELTNIIKNSSFKAVIYGGSAKDEVQIIDGNLGDLRDILKKGATFNRETPGVPIAYTTNF LKDNELAVIKNNSEYIETTSKAYTDGKINIDHSGGYVAQFNISWDEVNYDPEGNEIV QHKNWSENNKSKLAHFTSSIYLPGNARNINVYAKECTGLAWEWWRTVIDDRNLPL VKNRNISIWGTTLYPKYSNKVDNPIE (GenBank Accession No. P13128; SEQ ID NO: 12; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a vaccine disclosed herein. In another embodiment, an LLO AA sequence of methods and compositions disclosed herein comprises the sequence set forth in SEQ ID NO: 12. In another embodiment, the LLO AA sequence is a homologue of SEQ ID NO: 12. In another embodiment, the LLO AA sequence is a variant of SEQ ID NO: 12. In another embodiment, the LLO AA sequence is a fragment of SEQ ID NO: 12. In another embodiment, the LLO AA sequence is an isoform of SEQ ID NO: 12.

The LLO protein used in the compositions and methods disclosed herein comprise, in another embodiment, the sequence: M K K I M L V F I T L I L V S L P I A Q Q T E A K D A S A F N K E N S I S S V A P P A S P P A S P K T P I E K K H A D E I D K Y I Q G L D Y N K N N V L V Y H G D A V T N V P P R K G Y K D G N E Y I V V E K K K K S I N Q N N A D I Q V V N A I S S L T Y P G A L V K A N S E L V E N Q P D V L P V K R D S L T L S I D L P G M T N Q D N K I V V K N A T K S N V N N A V N T L V E R W N E K Y A Q A Y S N V S A K I D Y D D E M A Y S E S Q L I A K F G T A F K A V N N S L N V N F G A I S E G K M Q E E V I S F K Q I Y Y N V N V N E P T R P S R F F G K A V T K E Q L Q A L G V N A E N P P A Y I S S V A Y G R Q V Y L K L S T N S II S T K V K A A F D A A V S G K S V S G D V E L T N I I K N S S F K A V I Y G G S A K D E V Q I I D G N L G D L R D I L K K G A T F N R E T P G V P I A Y T T N F L K D N E L A V I K N N S E Y I E T T S K A Y T D G K I N I D H S G G Y V A Q F N I S W D E V N Y D P E G N E I V Q H K N W S E N N K S K L A H F T S S I Y L P G N A R N I N V Y A K E C T G L A W E W W R T V I D D R N L P L V K N R N I S I W G T T L Y P K Y S N K V D N P I E (SEQ ID NO: 13). In another embodiment, an LLO AA sequence of methods and compositions disclosed herein comprises the sequence set forth in SEQ ID NO: 13. In another embodiment, the LLO AA sequence is a homologue of SEQ ID NO: 13. In another embodiment, the LLO AA sequence is a variant of SEQ ID NO: 13. In another embodiment, the LLO AA sequence is a fragment of SEQ ID NO: 13. In another embodiment, the LLO AA sequence is an isoform of SEQ ID NO: 13.

In one embodiment, the amino acid sequence of the LLO polypeptide of the compositions and methods disclosed herein is from the Listeria monocytogenes 10403S strain, as set forth in Genbank Accession No.: ZP_01942330, EBA21833, or is encoded by the nucleic acid sequence as set forth in Genbank Accession No.: NZ_AARZ01000015 or AARZ01000015.1. In another embodiment, the LLO sequence for use in the compositions and methods disclosed herein is from Listeria monocytogenes, which in one embodiment, is the 4b F2365 strain (in one embodiment, Genbank accession number: YP_012823), the EGD-e strain (in one embodiment, Genbank accession number: NP_463733), or any other strain of Listeria monocytogenes known in the art.

In another embodiment, the LLO sequence for use in the compositions and methods disclosed herein is from Flavobacteriales bacterium HTCC2170 (in one embodiment, Genbank accession number: ZP_01106747 or EAR01433; in one embodiment, encoded by Genbank accession number: NZ_AAOC01000003). In one embodiment, proteins that are homologous to LLO in other species, such as alveolysin, which in one embodiment, is found in Paenibacillus alvei (in one embodiment, Genbank accession number: P23564 or AAA22224; in one embodiment, encoded by Genbank accession number: M62709) may be used in the compositions and methods disclosed herein. Other such homologous proteins are known in the art.

In another embodiment, homologues of LLO from other species, including known lysins, or fragments thereof may be used to create a fusion protein of LLO with an antigen of the compositions and methods disclosed herein, which in one embodiment, is HMW-MAA, and in another embodiment is a fragment of HMW-MAA.

In another embodiment, the LLO fragment of methods and compositions disclosed herein, is a PEST domain. In another embodiment, an LLO fragment that comprises a PEST sequence is utilized as part of a composition or in the methods disclosed herein.

In another embodiment, the LLO fragment does not contain the activation domain at the carboxy terminus. In another embodiment, the LLO fragment does not include cysteine 484. In another embodiment, the LLO fragment is a non-hemolytic fragment. In another embodiment, the LLO fragment is rendered non-hemolytic by deletion or mutation of the activation domain. In another embodiment, the LLO fragment is rendered non-hemolytic by deletion or mutation of cysteine 484. In another embodiment, an LLO sequence is rendered non-hemolytic by deletion or mutation in the cholesterol binding domain, as described in US Patent Publication Serial No. 2009/0081248. In another embodiment, an LLO sequence is rendered non-hemolytic by deletion or mutation at another location.

In one embodiment, the present invention provides a recombinant protein or polypeptide comprising a listeriolysin O (LLO) protein, wherein said LLO protein comprises a mutation of residues C484, W491, W492, or a combination thereof of the cholesterol-binding domain (CBD) of said LLO protein. In one embodiment, said C484, W491, and W492 residues are residues C484, W491, and W492 of SEQ ID NO: 12, while in another embodiment, they are corresponding residues as can be deduced using sequence alignments, as is known to one of skill in the art. In one embodiment, residues C484, W491, and W492 are mutated. In one embodiment, a mutation is a substitution, in another embodiment, a deletion. In one embodiment, the entire CBD is mutated, while in another embodiment, portions of the CBD are mutated, while in another embodiment, only specific residues within the CBD are mutated.

In another embodiment, the present invention provides a recombinant protein or polypeptide comprising (a) a mutated LLO protein, wherein the mutated LLO protein contains an internal deletion, the internal deletion comprising the cholesterol-binding domain of the mutated LLO protein; and (b) a heterologous peptide of interest. In another embodiment, the sequence of the cholesterol-binding domain is ECTGLAWEWWR, which is set forth in SEQ ID NO: 42). In another embodiment, the internal deletion is an 11-50 amino acid internal deletion. In another embodiment, the internal deletion is inactivating with regard to the hemolytic activity of the recombinant protein or polypeptide. In another embodiment, the recombinant protein or polypeptide exhibits a reduction in hemolytic activity relative to wild-type LLO.

In another embodiment, the present invention provides a recombinant protein or polypeptide comprising (a) a mutated LLO protein, wherein the mutated LLO protein contains an internal deletion, the internal deletion comprising a fragment of the cholesterol-binding domain of the mutated LLO protein; and (b) a heterologous peptide of interest. In another embodiment, the internal deletion is a 1-11 amino acid internal deletion. In another embodiment, the sequence of the cholesterol-binding domain is set forth in SEQ ID NO: 42. In another embodiment, the internal deletion is inactivating with regard to the hemolytic activity of the recombinant protein or polypeptide. In another embodiment, the recombinant protein or polypeptide exhibits a reduction in hemolytic activity relative to wild-type LLO.

In another embodiment, a peptide of the present invention is a fusion peptide. In another embodiment, “fusion peptide” refers to a peptide or polypeptide comprising two or more proteins linked together by peptide bonds or other chemical bonds. In another embodiment, the proteins are linked together directly by a peptide or other chemical bond. In another embodiment, the proteins are linked together with one or more AA (e.g. a “spacer”) between the two or more proteins.

The length of the internal deletion of methods and compositions of the present invention is, in another embodiment, 1-50 AA. In another embodiment, the length is 1-11 AA. In another embodiment, the length is 2-11 AA. In another embodiment, the length is 3-11 AA. In another embodiment, the length is 4-11 AA. In another embodiment, the length is 5-11 AA. In another embodiment, the length is 6-11 AA. In another embodiment, the length is 7-11 AA. In another embodiment, the length is 8-11 AA. In another embodiment, the length is 9-11 AA. In another embodiment, the length is 10-11 AA. In another embodiment, the length is 1-2 AA. In another embodiment, the length is 1-3 AA. In another embodiment, the length is 1-4 AA. In another embodiment, the length is 1-5 AA. In another embodiment, the length is 1-6 AA. In another embodiment, the length is 1-7 AA. In another embodiment, the length is 1-8 AA. In another embodiment, the length is 1-9 AA. In another embodiment, the length is 1-10 AA. In another embodiment, the length is 2-3 AA. In another embodiment, the length is 2-4 AA. In another embodiment, the length is 2-5 AA. In another embodiment, the length is 2-6 AA. In another embodiment, the length is 2-7 AA. In another embodiment, the length is 2-8 AA. In another embodiment, the length is 2-9 AA. In another embodiment, the length is 2-10 AA. In another embodiment, the length is 3-4 AA. In another embodiment, the length is 3-5 AA. In another embodiment, the length is 3-6 AA. In another embodiment, the length is 3-7 AA. In another embodiment, the length is 3-8 AA. In another embodiment, the length is 3-9 AA. In another embodiment, the length is 3-10 AA. In another embodiment, the length is 11-50 AA. In another embodiment, the length is 12-50 AA. In another embodiment, the length is 11-15 AA. In another embodiment, the length is 11-20 AA. In another embodiment, the length is 11-25 AA. In another embodiment, the length is 11-30 AA. In another embodiment, the length is 11-35 AA. In another embodiment, the length is 11-40 AA. In another embodiment, the length is 11-60 AA. In another embodiment, the length is 11-70 AA. In another embodiment, the length is 11-80 AA. In another embodiment, the length is 11-90 AA. In another embodiment, the length is 11-100 AA. In another embodiment, the length is 11-150 AA. In another embodiment, the length is 15-20 AA. In another embodiment, the length is 15-25 AA. In another embodiment, the length is 15-30 AA. In another embodiment, the length is 15-35 AA. In another embodiment, the length is 15-40 AA. In another embodiment, the length is 15-60 AA. In another embodiment, the length is 15-70 AA. In another embodiment, the length is 15-80 AA. In another embodiment, the length is 15-90 AA. In another embodiment, the length is 15-100 AA. In another embodiment, the length is 15-150 AA. In another embodiment, the length is 20-25 AA. In another embodiment, the length is 20-30 AA. In another embodiment, the length is 20-35 AA. In another embodiment, the length is 20-40 AA. In another embodiment, the length is 20-60 AA. In another embodiment, the length is 20-70 AA. In another embodiment, the length is 20-80 AA. In another embodiment, the length is 20-90 AA. In another embodiment, the length is 20-100 AA. In another embodiment, the length is 20-150 AA. In another embodiment, the length is 30-35 AA. In another embodiment, the length is 30-40 AA. In another embodiment, the length is 30-60 AA. In another embodiment, the length is 30-70 AA. In another embodiment, the length is 30-80 AA. In another embodiment, the length is 30-90 AA. In another embodiment, the length is 30-100 AA. In another embodiment, the length is 30-150 AA.

In another embodiment, the mutated LLO protein of the present invention that comprises an internal deletion is full length except for the internal deletion. In another embodiment, the mutated LLO protein comprises an additional internal deletion. In another embodiment, the mutated LLO protein comprises more than one additional internal deletion. In another embodiment, the mutated LLO protein is truncated from the C-terminal end. In another embodiment, the mutated LLO protein is truncated from the N-terminal end.

The internal deletion of methods and compositions of the present invention comprises, in another embodiment, residue C484 of SEQ ID NO: 12. In another embodiment, the internal deletion comprises a corresponding cysteine residue of a homologous LLO protein. In another embodiment, the internal deletion comprises residue W491 of SEQ ID NO: 12. In another embodiment, the internal deletion comprises a corresponding tryptophan residue of a homologous LLO protein. In another embodiment, the internal deletion comprises residue W492 of SEQ ID NO: 12. In another embodiment, the internal deletion comprises a corresponding tryptophan residue of a homologous LLO protein. Methods for identifying corresponding residues of a homologous protein are well known in the art, and include, for example, sequence alignment.

In another embodiment, the internal deletion comprises residues C484 and W491. In another embodiment, the internal deletion comprises residues C484 and W492. In another embodiment, the internal deletion comprises residues W491 and W492. In another embodiment, the internal deletion comprises residues C484, W491, and W492.

In one embodiment, the present invention provides a recombinant protein or polypeptide comprising a mutated LLO protein or fragment thereof, wherein the mutated LLO protein or fragment thereof contains a substitution of a non-LLO peptide for a mutated region of the mutated LLO protein or fragment thereof, the mutated region comprising a residue selected from C484, W491, and W492. In another embodiment, the LLO fragment is an N-terminal LLO fragment. In another embodiment, the LLO fragment is at least 492 amino acids (AA) long. In another embodiment, the LLO fragment is 492-528 AA long. In another embodiment, the non-LLO peptide is 1-50 amino acids long. In another embodiment, the mutated region is 1-50 amino acids long. In another embodiment, the non-LLO peptide is the same length as the mutated region. In another embodiment, the non-LLO peptide has a length different from the mutated region. In another embodiment, the substitution is an inactivating mutation with respect to hemolytic activity. In another embodiment, the recombinant protein or polypeptide exhibits a reduction in hemolytic activity relative to wild-type LLO. In another embodiment, the recombinant protein or polypeptide is non-hemolytic.

In another embodiment, the internal deletion of methods and compositions of the present invention comprises the CBD of the mutated LLO protein or fragment thereof. For example, an internal deletion consisting of residues 470-500, 470-510, or 480-500 of SEQ ID NO: 12 comprises the CBD thereof (residues 483-493). In another embodiment, the internal deletion is a fragment of the CBD of the mutated LLO protein or fragment thereof. For example, residues 484-492, 485-490, and 486-488 are all fragments of the CBD of SEQ ID NO: 12. In another embodiment, the internal deletion overlaps the CBD of the mutated LLO protein or fragment thereof. For example, an internal deletion consisting of residues 470-490, 480-488, 490-500, or 486-510 of SEQ ID NO: 12 comprises the CBD thereof.

“Hemolytic” refers, in another embodiment, to ability to lyse a eukaryotic cell. In another embodiment, the eukaryotic cell is a red blood cell. In another embodiment, the eukaryotic cell is any other type of eukaryotic cell known in the art. In another embodiment, hemolytic activity is measured at an acidic pH. In another embodiment, hemolytic activity is measured at physiologic pH. In another embodiment, hemolytic activity is measured at pH 5.5. In another embodiment, hemolytic activity is measured at pH 7.4. In another embodiment, hemolytic activity is measured at any other pH known in the art.

In another embodiment, a recombinant protein or polypeptide of methods and compositions of the present invention exhibits a greater than 100-fold reduction in hemolytic activity relative to wild-type LLO. In another embodiment, the recombinant protein or polypeptide exhibits a greater than 50-fold reduction in hemolytic activity. In another embodiment, the reduction is greater than 30-fold. In another embodiment, the reduction is greater than 40-fold. In another embodiment, the reduction is greater than 60-fold. In another embodiment, the reduction is greater than 70-fold. In another embodiment, the reduction is greater than 80-fold. In another embodiment, the reduction is greater than 90-fold. In another embodiment, the reduction is greater than 120-fold. In another embodiment, the reduction is greater than 150-fold. In another embodiment, the reduction is greater than 200-fold. In another embodiment, the reduction is greater than 250-fold. In another embodiment, the reduction is greater than 300-fold. In another embodiment, the reduction is greater than 400-fold. In another embodiment, the reduction is greater than 500-fold. In another embodiment, the reduction is greater than 600-fold. In another embodiment, the reduction is greater than 800-fold. In another embodiment, the reduction is greater than 1000-fold. In another embodiment, the reduction is greater than 1200-fold. In another embodiment, the reduction is greater than 1500-fold. In another embodiment, the reduction is greater than 2000-fold. In another embodiment, the reduction is greater than 3000-fold. In another embodiment, the reduction is greater than 5000-fold.

In another embodiment, the reduction is at least 100-fold. In another embodiment, the reduction is at least 50-fold. In another embodiment, the reduction is at least 30-fold. In another embodiment, the reduction is at least 40-fold. In another embodiment, the reduction is at least 60-fold. In another embodiment, the reduction is at least 70-fold. In another embodiment, the reduction is at least 80-fold. In another embodiment, the reduction is at least 90-fold. In another embodiment, the reduction is at least 120-fold. In another embodiment, the reduction is at least 150-fold. In another embodiment, the reduction is at least 200-fold. In another embodiment, the reduction is at least 250-fold. In another embodiment, the reduction is at least 300-fold. In another embodiment, the reduction is at least 400-fold. In another embodiment, the reduction is at least 500-fold. In another embodiment, the reduction is at least 600-fold. In another embodiment, the reduction is at least 800-fold. In another embodiment, the reduction is at least 1000-fold. In another embodiment, the reduction is at least 1200-fold. In another embodiment, the reduction is at least 1500-fold. In another embodiment, the reduction is at least 2000-fold. In another embodiment, the reduction is at least 3000-fold. In another embodiment, the reduction is at least 5000-fold.

Methods of determining hemolytic activity are well known in the art, and are described, for example, in the Examples herein, and in Portnoy DA et al, (J Exp Med Vol 167:1459-1471, 1988) and Dancz CE et al (J Bacteriol. 184: 5935-5945, 2002).

“Inactivating mutation” with respect to hemolytic activity refers, in another embodiment, to a mutation that abolishes detectable hemolytic activity. In another embodiment, the term refers to a mutation that abolishes hemolytic activity at pH 5.5. In another embodiment, the term refers to a mutation that abolishes hemolytic activity at pH 7.4. In another embodiment, the term refers to a mutation that significantly reduces hemolytic activity at pH 5.5. In another embodiment, the term refers to a mutation that significantly reduces hemolytic activity at pH 7.4. In another embodiment, the term refers to a mutation that significantly reduces hemolytic activity at pH 5.5. In another embodiment, the term refers to any other type of inactivating mutation with respect to hemolytic activity.

In another embodiment, the sequence of the cholesterol-binding domain of methods and compositions of the present invention is set forth in SEQ ID NO: 42. In another embodiment, the CBD is any other LLO CBD known in the art.

In another embodiment, the LLO fragment consists of about the first 441 AA of the LLO protein. In another embodiment, the LLO fragment comprises about the first 400-441 AA of the 529 AA full length LLO protein. In another embodiment, the LLO fragment corresponds to AA 1-441 of an LLO protein disclosed herein. In another embodiment, the LLO fragment consists of about the first 420 AA of LLO. In another embodiment, the LLO fragment corresponds to AA 1-420 of an LLO protein disclosed herein. In another embodiment, the LLO fragment consists of about AA 20-442 of LLO. In another embodiment, the LLO fragment corresponds to AA 20-442 of an LLO protein disclosed herein. In another embodiment, any ALLO without the activation domain comprising cysteine 484, and in particular without cysteine 484, are suitable for methods and compositions disclosed herein.

In another embodiment, the LLO fragment corresponds to the first 400 AA of an LLO protein. In another embodiment, the LLO fragment corresponds to the first 300 AA of an LLO protein. In another embodiment, the LLO fragment corresponds to the first 200 AA of an LLO protein. In another embodiment, the LLO fragment corresponds to the first 100 AA of an LLO protein. In another embodiment, the LLO fragment corresponds to the first 50 AA of an LLO protein, which in one embodiment, comprises one or more PEST sequences.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein.

In another embodiment, a recombinant Listeria strain of the methods and compositions disclosed herein comprise a nucleic acid molecule operably integrated into the Listeria genome as an open reading frame with an endogenous ActA sequence. In another embodiment, an episomal expression vector disclosed herein comprises a fusion protein comprising an antigen fused to an ActA or a truncated ActA.

In another embodiment, a recombinant Listeria strain of the methods and compositions disclosed herein comprise a nucleic acid molecule operably integrated into the Listeria genome as an open reading frame with an endogenous ActA sequence. In another embodiment, a recombinant Listeria strain of the methods and compositions disclosed herein comprise an episomal expression vector comprising a nucleic acid molecule encoding fusion protein comprising an antigen fused to an ActA or a truncated ActA. In one embodiment, the expression and secretion of the antigen is under the control of an actA promoter and ActA signal sequence and it is expressed as fusion to 1-233 amino acids of ActA (truncated ActA or tActA). In another embodiment, the truncated ActA consists of the first 390 amino acids of the wild type ActA protein as described in U.S. Pat. Ser. No. 7,655,238, which is incorporated by reference herein in its entirety. In another embodiment, the truncated ActA is an ActA-N100 or a modified version thereof (referred to as ActA-N100*) in which a PEST motif has been deleted and containing the nonconservative QDNKR substitution as described in US Patent Publication Serial No. 2014/0186387.

In one embodiment, the antigen is survivin, while in another embodiment, it's an immunogenic fragment of survivin. In another embodiment, it is an epitope of survivin. In another embodiment, the survivin epitope is an HLA-A2 suvivin epitope has the sequence set forth LMLGEFLKL (SEQ ID NO: 14).

In one embodiment, an antigen of the methods and compositions disclosed herein is fused to an ActA protein, which in one embodiment, is an N-terminal fragment of an ActA protein, which in one embodiment, comprises or consists of the first 390 AA of ActA, in another embodiment, the first 418 AA of ActA, in another embodiment, the first 50 AA of ActA, in another embodiment, the first 100 AA of ActA, which in one embodiment, comprise a PEST sequence such as that provided in SEQ ID NO: 2. In another embodiment, an N-terminal fragment of an ActA protein utilized in methods and compositions disclosed herein comprises or consists of the first 150 AA of ActA, in another embodiment, the first approximately 200 AA of ActA, which in one embodiment comprises 2 PEST sequences as described herein. In another embodiment, an N-terminal fragment of an ActA protein utilized in methods and compositions disclosed herein comprises or consists of the first 250 AA of ActA, in another embodiment, the first 300 AA of ActA. In another embodiment, the ActA fragment contains residues of a homologous ActA protein that correspond to one of the above AA ranges. The residue numbers need not, in another embodiment, correspond exactly with the residue numbers enumerated above; e.g. if the homologous ActA protein has an insertion or deletion, relative to an ActA protein utilized herein, then the residue numbers can be adjusted accordingly, as would be routine to a skilled artisan using sequence alignment tools such as NCBI BLAST that are well-known in the art.

In another embodiment, the N-terminal portion of the ActA protein comprises 1, 2, 3, or 4 PEST sequences, which in one embodiment are the PEST sequences specifically mentioned herein, or their homologs, as described herein or other PEST sequences as can be determined using the methods and algorithms described herein or by using alternative methods known in the art.

An N-terminal fragment of an ActA protein utilized in methods and compositions disclosed herein has, in another embodiment, the sequence set forth in SEQ ID NO: 15: MRAMMVVFITANCITINPDIIFAATDSEDSSLNTDEWEEEKTEEQPSEVNTGPRYETA REVSSRDIKELEKSNKVRNTNKADLIAMLKEKAEKGPNINNNNSEQTENAAINEEAS GADRPAIQVERRHPGLPSDSAAEIKKRRKAIASSDSELESLTYPDKPTKVNKKKVAK ESVADASESDLDSSMQSADESSPQPLKANQQPFFPKVFKKIKDAGKWVRDKIDENPE VKKAIVDKSAGLIDQLLTKKKSEEVNASDFPPPPTDEELRLALPETPMLLGFNAPATS EPSSFEFPPPPTDEELRLALPETPMLLGFNAPATSEPSSFEFPPPPTEDELEIIRETASSLD SSFTRGDLASLRNAINRIISQNFSDFPPIPTEEELNGRGGRP (SEQ ID NO: 15). In another embodiment, the ActA fragment comprises the sequence set forth in SEQ ID NO: 15. In another embodiment, the ActA fragment is any other ActA fragment known in the art. In another embodiment, the ActA protein is a homologue of SEQ ID NO: 15. In another embodiment, the ActA protein is a variant of SEQ ID NO: 15. In another embodiment, the ActA protein is an isoform of SEQ ID NO: 15. In another embodiment, the ActA protein is a fragment of SEQ ID NO: 15. In another embodiment, the ActA protein is a fragment of a homologue of SEQ ID NO: 15. In another embodiment, the ActA protein is a fragment of a variant of SEQ ID NO: 15. In another embodiment, the ActA protein is a fragment of an isoform of SEQ ID NO: 15.

In another embodiment, the recombinant nucleotide encoding a fragment of an ActA protein comprises the sequence set forth in SEQ ID NO: 16: atgcgtgcgatgatggtggttttcattactgccaattgcattacgattaaccccgacataatatttgcagcgacagatagcgaagattcta gtctaaacacagatgaatgggaagaagaaaaaacagaagagcaaccaagcgaggtaaatacgggaccaagatacgaaactgcac gtgaagtaagttcacgtgatattaaagaactagaaaaatcgaataaagtgagaaatacgaacaaagcagacctaatagcaatgttgaa agaaaaagcagaaaaaggtccaaatatcaataataacaacagtgaacaaactgagaatgcggctataaatgaagaggcttcaggag ccgaccgaccagctatacaagtggagcgtcgtcatccaggattgccatcggatagcgcagcggaaattaaaaaaagaaggaaagc catagcatcatcggatagtgagcttgaaagccttacttatccggataaaccaacaaaagtaaataagaaaaaagtggcgaaagagtc agttgcggatgcttctgaaagtgacttagattctagcatgcagtcagcagatgagtcttcaccacaacctttaaaagcaaaccaacaac catttttccctaaagtatttaaaaaaataaaagatgcggggaaatgggtacgtgataaaatcgacgaaaatcctgaagtaaagaaagc gattgttgataaaagtgcagggttaattgaccaattattaaccaaaaagaaaagtgaagaggtaaatgcttcggacttcccgccaccac ctacggatgaagagttaagacttgctttgccagagacaccaatgcttcttggttttaatgctcctgctac atcagaaccgagctcattcg aatttccaccaccacctacggatgaagagttaagacttgctttgccagagacgccaatgcttcttggttttaatgctcctgctacatcgga accgagctcgttcgaatttccaccgcctccaacagaagatgaactagaaatcatccgggaaacagcatcctcgctagattctagtttta caagaggggatttagctagtttgagaaatgctattaatcgccatagtcaaaatttctctgatttcccaccaatcccaacagaagaagagt tgaacgggagaggcggtagacca (SEQ ID NO: 16). In another embodiment, the recombinant nucleotide has the sequence set forth in SEQ ID NO: 16. In another embodiment, the recombinant nucleotide comprises any other sequence that encodes a fragment of an ActA protein.

An N-terminal fragment of an ActA protein utilized in methods and compositions disclosed herein has, in another embodiment, the sequence set forth in SEQ ID NO: 17: MRAMMVVFITANCITINPDIIFAATDSEDSSLNTDEWEEEKTEEQPSEVNTGPRYETA REVSSRDIEELEKSNKVKNTNKADLIAMLKAKAEKGPNNNNNNGEQTGNVAINEEA SGVDRPTLQVERRHPGLSSDSAAEIKKRRKAIASSDSELESLTYPDKPTKANKRKVA KESVVDASESDLDSSMQSADESTPQPLKANQKPFFPKVFKKIKDAGKWVRDKIDEN PEVKKAIVDKSAGLIDQLLTKKKSEEVNASDFPPPPTDEELRLALPETPMLLGFNAPT PSEPSSFEFPPPPTDEELRLALPETPMLLGFNAPATSEPSSFEFPPPPTEDELEIMRETAP SLDSSFTSGDLASLRSAINRHSENFSDFPLIPTEEELNGRGGRP (SEQ ID NO: 17), which in one embodiment is the first 390 AA for ActA from Listeria monocytogenes, strain 10403S. In another embodiment, the ActA fragment comprises the sequence set forth in SEQ ID NO: 17. In another embodiment, the ActA fragment is any other ActA fragment known in the art. In another embodiment, the ActA protein is a homologue of SEQ ID NO: 17. In another embodiment, the ActA protein is a variant of SEQ ID NO: 17. In another embodiment, the ActA protein is an isoform of SEQ ID NO: 17. In another embodiment, the ActA protein is a fragment of SEQ ID NO: 17. In another embodiment, the ActA protein is a fragment of a homologue of SEQ ID NO: 17. In another embodiment, the ActA protein is a fragment of a variant of SEQ ID NO: 17. In another embodiment, the ActA protein is a fragment of an isoform of SEQ ID NO: 17.

In another embodiment, the recombinant nucleotide encoding a fragment of an ActA protein comprises the sequence set forth in SEQ ID NO: 18: atgcgtgcgatgatggtagttttcattactgccaactgcattacgattaaccccgacataatatttgcagcgacagatagcgaagattcca gtctaaacacagatgaatgggaagaagaaaaaacagaagagcagccaagcgaggtaaatacgggaccaagatacgaaactgcac gtgaagtaagttcacgtgatattgaggaactagaaaaatcgaataaagtgaaaaatacgaacaaagcagacctaatagcaatgttgaa agcaaaagcagagaaaggtccgaataacaataataacaacggtgagcaaacaggaaatgtggctataaatgaagaggcttcaggag tcgaccgaccaactctgcaagtggagcgtcgtcatccaggtctgtcatcggatagcgcagcggaaattaaaaaaagaagaaaagcc atagcgtcgtcggatagtgagcttgaaagccttacttatccagataaaccaacaaaagcaaataagagaaaagtggcgaaagagtca gttgtggatgcttctgaaagtgacttagattctagcatgcagtcagcagacgagtctacaccacaacctttaaaagcaaatcaaaaacca tttttccctaaagtatttaaaaaaataaaagatgcggggaaatgggtacgtgataaaatcgacgaaaatcctgaagtaaagaaagcgatt gttgataaaagtgcagggttaattgaccaattattaaccaaaaagaaaagtgaagaggtaaatgcttcggacttcccgccaccacctac ggatgaagagttaagacttgctttgccagagacaccgatgcttctcggttttaatgctcctactccatcggaaccgagctcattcgaatttc cgccgccacctacggatgaagagttaagacttgattgccagagacgccaatgatcttggttttaatgctcctgctacatcggaaccga gctcattcgaatttccaccgcctccaacagaagatgaactagaaattatgcgggaaacagcaccttcgctagattctagttttacaagcg gggatttagctagtttgagaagtgctattaatcgccatagcgaaaatttctctgatttcccactaatcccaacagaagaagagttgaacgg gagaggcggtagacca (SEQ ID NO: 18), which in one embodiment, is the first 1170 nucleotides encoding ActA in Listeria monocytogenes 10403S strain. In another embodiment, the recombinant nucleotide has the sequence set forth in SEQ ID NO: 18. In another embodiment, the recombinant nucleotide comprises any other sequence that encodes a fragment of an ActA protein.

In another embodiment, the ActA fragment is another ActA fragment known in the art, which in one embodiment, is any fragment comprising a PEST sequence. Thus, in one embodiment, the ActA fragment is amino acids 1-100 of the ActA sequence. In another embodiment, the ActA fragment is amino acids 1-200 of the ActA sequence. In another embodiment, the ActA fragment is amino acids 200-300 of the ActA sequence. In another embodiment, the ActA fragment is amino acids 300-400 of the ActA sequence. In another embodiment, the ActA fragment is amino acids 1-300 of the ActA sequence. In another embodiment, a recombinant nucleotide disclosed herein comprises any other sequence that encodes a fragment of an ActA protein. In another embodiment, the recombinant nucleotide comprises any other sequence that encodes an entire ActA protein. In another embodiment, the actA fragment comprises (a) amino acids 1-59 of actA, (b) an inactivating mutation in, deletion of, or truncation prior to, at least one domain for acta-mediated regulation of the host cell cytoskeleton. In some embodiments the ActA comprises more than the first 59 amino acids of ActA. In some embodiments, the modified actA is actA-N100 as described in US Patent Publication Serial No. 2007/0207170, which is hereby incorporated by reference in its entirety.

In one embodiment, the ActA sequence for use in the compositions and methods provided herein is from Listeria monocytogenes, which in one embodiment, is the EGD strain, the 10403S strain (Genbank accession number: DQ054585) the NICPBP 54002 strain (Genbank accession number: EU394959), the S3 strain (Genbank accession number: EU394960), the NCTC 5348 strain (Genbank accession number: EU394961), the NICPBP 54006 strain (Genbank accession number: EU394962), the M7 strain (Genbank accession number: EU394963), the S19 strain (Genbank accession number: EU394964), or any other strain of Listeria monocytogenes which is known in the art.

In one embodiment, the sequence of the deleted actA region in the strain, LmddΔactA is as follows:

(SEQ ID NO: 19)
gcgccaaatcattggttgattggtgaggatgtctgtgtgcgtgggtcgcg
agatgggcgaataagaagcattaaagatcctgacaaatataatcaagcgg
ctcatatgaaagattacgaatcgcttccactcacagaggaaggcgactgg
ggcggagttcattataatagtggtatcccgaataaagcagcctataatac
tatcactaaacttggaaaagaaaaaacagaacagctttattttcgcgcct
taaagtactatttaacgaaaaaatcccagtttaccgatgcgaaaaaagcg
cttcaacaagcagcgaaagatttatatggtgaagatgcttctaaaaaagt
tgctgaagcttgggaagcagttggggttaactgattaacaaatgttagag
aaaaattaattctccaagtgatattcttaaaataattcatgaatattttt
tcttatattagctaattaagaagataactaactgctaatccaatttttaa
cggaacaaattagtgaaaatgaaggccgaattttccttgttctaaaaagg
ttgtattagcgtatcacgaggagggagtataagtgggattaaacagattt
atgcgtgcgatgatggtggttttcattactgccaattgcattacgattaa
ccccgacgtcgacccatacgacgttaattatgcaatgttagctattggcg
tgactattaggggcgatatcaaaattattcaattaagaaaaaataattaa
aaacacagaacgaaagaaaaagtgaggtgaatgatatgaaattcaaaaag
gtggttctaggtatgtgcttgatcgcaagtgttctagtctttccggtaac
gataaaagcaaatgcctgttgtgatgaatacttacaaacacccgcagctc
cgcatgatattgacagcaaattaccacataaacttagttggtccgcggat
aacccgacaaatactgacgtaaatacgcactattggctttttaaacaagc
ggaaaaaatactagctaaagatgtaaatcatatgcgagctaatttaatga
atgaacttaaaaaattcgataaacaaatagctcaaggaatatatgatgcg
gatcataaaaatccatattatgatactagtacatttttatctcattttta
taatcctgatagagataatacttatttgccgggttttgctaatgcgaaaa
taacaggagcaaagtatttcaatcaatcggtgactgattaccgagaaggg
aa.

In one embodiment, the underlined region contains actA sequence element that is present in the LmddΔactA strain. In one embodiment, the bold sequence gtcgac represent the site of junction of the N-T and C-T sequence.

In one embodiment, the recombinant Listeria strain of the compositions and methods provided herein comprise a nucleic acid molecule that encodes a survivin antigen, or in another embodiment, a fragment of survivin.

In another embodiment, the mouse survivin protein has the following amino acid (AA) sequence:

(SEQ ID NO: 20)
MGAPALPQIWQLYLKNYRIATFKNWPFLEDCACAPERMAEAGFIHCPTE
NEPDLAQCFFCFKELEGWEPDDNPIEEHRKHSPGCAFLTVKKQMEELTV
SEFLKLDRQRAKNKIAKETNNKQKEFEETAKTTRQSIEQLAA.

In another embodiment, an survivin amino acid sequence of methods and compositions disclosed herein comprises the sequence set forth in SEQ ID No: 20. In another embodiment, the survivin sequence is a homologue of SEQ ID No: 20. In another embodiment, the survivin AA sequence is a variant of SEQ ID No: 20. In another embodiment, the survivin AA sequence is a fragment of SEQ ID No: 20. In another embodiment, the survivin AA sequence is an isoform of SEQ ID No: 20.

In another embodiment, the human survivin protein has the following amino acid sequence:

(SEQ ID NO: 21)
GAPTLPPAWQ PFLKDHRIST FKNWPFLEGC ACAPERMAEA
GFIHCPTENEPDLAQCFFCFKELEGWEPDDDPIEEHKKHSSGCAFLSVKK
QFEELTLGEFLKLDRERAKNKIAKETNNKKKEFEETAKKVRRAIEQLAAM
D.

In another embodiment, an survivin amino acid sequence of methods and compositions disclosed herein comprises the sequence set forth in SEQ ID No: 21. In another embodiment, the survivin sequence is a homologue of SEQ ID No: 21. In another embodiment, the survivin AA sequence is a variant of SEQ ID No: 21. In another embodiment, the survivinAA sequence is a fragment of SEQ ID No: 21. In another embodiment, the survivin AA sequence is an isoform of SEQ ID No: 21.

In another embodiment, the mouse survivin protein is encoded by the following nucleic acid sequence:GGGAGC TCCGGCGCTG CCCCAGATCT GGCAGCTGTA CCTCAAGAAC TACCGCATCG CCACCTTCAA GAACTGGCCC TTCCTGGAGG ACTGCGCCTG CGCCCCAGAG CGAATGGCGG AGGCTGGCTT CATCCACTGC CCTACCGAGA ACGAGCCTGA TTTGGCCCAG TGTTTTTTCT GCTTTAAGGA ATTGGAAGGC TGGGAACCCG ATGACAACCC GATAGAGGAG CATAGAAAGC ACTCCCCTGG CTGCGCCTTC CTCACTGTCA AGAAGCAGAT GGAAGAACTA ACCGTCAGTG AATTCTTGAA ACTGGACAGA CAGAGAGCCA AGAACAAAAT TGCAAAGGAG ACCAACAACA AGCAAAAAGA GTTTGAAGAG ACTGCAAAGA CTACCCGTCA GTCAATTGAGCAGCTGGCTGCCTAA(SEQ ID No: 22. In another embodiment, the recombinant nucleotide has the sequence set forth in SEQ ID NO: 22. In another embodiment, an survivin-encoding nucleotide of methods and compositions disclosed herein comprises the sequence set forth in SEQ ID No: 22. In another embodiment, the survivin-encoding nucleotide is a homologue of SEQ ID No: 22. In another embodiment, the survivin-encoding nucleotide is a variant of SEQ ID No: 22. In another embodiment, the survivin-encoding nucleotide is a fragment of SEQ ID No: 22. In another embodiment, the survivin-encoding nucleotide is an isoform of SEQ ID No: 22.

In another embodiment, a human survivin protein is encoded by the following nucleic acid sequence: GGTGCCCCGACGTTGCCCCCTGCCTGGCAGCCCTTTCTCAAGGACCACCGCATCT CTACATTCAAGAACTGGCCCTTCTTGGAGGGCTGCGCCTGCGCCCCGGAGCGGA TGGCCGAGGCTGGCTTCATCCACTGCCCCACTGAGAACGAGCCAGACTTGGCCC AGTGTTTCTTCTGCTTCAAGGAGCTGGAAGGCTGGGAGCCAGATGACGACCCCA TAGAGGAACATAAAAAGCATTCGTCCGGTTGCGCTTTCCTTTCTGTCAAGAAGCA GTTTGAAGAATTAACCCTTGGTGAATTTTTGAAACTGGACAGAGAAAGAGCCAA GAACAAAATTGCAAAGGAAACCAACAATAAGAAGAAAGAATTTGAGGAAACTG CGAAGAAAGTGCGCCGTGCCATCGAGCAGCTGGCTGCCATGGATTGA (SEQ ID NO: 23). In another embodiment, the recombinant nucleotide has the sequence set forth in SEQ ID NO: 23. In another embodiment, an survivin-encoding nucleotide of methods and compositions disclosed herein comprises the sequence set forth in SEQ ID No: 23. In another embodiment, the survivin-encoding nucleotide is a homologue of SEQ ID No: 23. In another embodiment, the survivin-encoding nucleotide is a variant of SEQ ID No: 23. In another embodiment, the survivin-encoding nucleotide is a fragment of SEQ ID No: 23. In another embodiment, the survivin-encoding nucleotide is an isoform of SEQ ID No: 23.

In another embodiment, the survivin protein of methods and compositions disclosed herein has an AA sequence set forth in a GenBank entry having an Accession Numbers selected from AAX29118.1, 1F3H_A, and CAG46540.1 and NP_033819.1. In another embodiment, the survivin protein is encoded by a nucleotide sequence set forth in one of the above GenBank entries. In another embodiment, the survivin protein comprises a sequence set forth in one of the above GenBank entries. In another embodiment, the survivin protein is a homologue of a sequence set forth in one of the above GenBank entries. In another embodiment, the survivin protein is a variant of a sequence set forth in one of the above GenBank entries. In another embodiment, the survivin protein is a fragment of a sequence set forth in one of the above GenBank entries. In another embodiment, the survivin protein is an isoform of a sequence set forth in one of the above GenBank entries.

In another embodiment, the recombinant Listeria of the compositions and methods disclosed herein comprise a plasmid that encodes a recombinant polypeptide that is, in one embodiment, angiogenic, and in another embodiment, antigenic. In one embodiment, the polypeptide is survivin, and in another embodiment, the polypeptide is a survivin fragment. In another embodiment, the plasmid further encodes a polypeptide comprising a PEST sequence. In one embodiment, the survivin fragment of methods and compositions provided herein is fused to the polypeptide comprising a PEST sequence. In one embodiment, the polypeptide comprising a PEST sequence enhances the immunogenicity of the antigenic or angiogenic polypeptide when fused thereto. In another embodiment, the survivin fragment is embedded within the peptide comprising a PEST sequence. In another embodiment, an survivin-derived peptide is incorporated into an LLO fragment, ActA protein or fragment, or PEST sequence.

The polypeptide comprising a PEST sequence is, in one embodiment, a listeriolysin (LLO) oligopeptide. In another embodiment, the polypeptide comprising a PEST sequence is an ActA oligopeptide. In another embodiment, the polypeptide comprising a PEST sequence is a PEST oligopeptide. In one embodiment, fusion to LLO, ActA, PEST sequences and fragments thereof enhances the cell-mediated immunogenicity of antigens. In one embodiment, fusion to LLO, ActA, PEST sequences and fragments thereof enhances the cell-mediated immunogenicity of antigens in a variety of expression systems. In another embodiment, the polypeptide comprising a PEST sequence is any other immunogenic polypeptide comprising a PEST sequence known in the art.

In one embodiment, the recombinant Listeria strain of the compositions and methods disclosed herein express a heterologous antigenic polypeptide that is expressed by a tumor cell. In one embodiment, the recombinant Listeria strain of the compositions and methods provided herein comprise a first and second nucleic acid molecule each comprising an open reading frame that encodes a heterologous antigen fused to a PEST-containing sequence such as a truncated LLO, a truncated ActA or a PEST peptide.

In another embodiment, the heterologous antigen provided herein is a tumor-associated antigen, which in one embodiment, is one of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, a MART1 antigen associated with melanoma, or the PSA antigen associated with prostate cancer. In another embodiment, the antigen for the compositions and methods disclosed herein are melanoma-associated antigens, which in one embodiment are TRP-2, MAGE-1, MAGE-3, gp-100, tyrosinase, HSP-70, beta-HCG, or a combination thereof.

In another embodiment, the antigen is HPV-E7. In another embodiment, the antigen is NY-ESO-1. In another embodiment, the antigen is telomerase (TERT). In another embodiment, the antigen is SCCE. In another embodiment, the antigen is CEA. In another embodiment, the antigen is LMP-1. In another embodiment, the antigen is PSMA. In another embodiment, the antigen is prostate stem cell antigen (PSCA). In another embodiment, the antigen is WT-1. In another embodiment, the antigen is HIV-1 Gag. In another embodiment, the antigen is Proteinase 3. In another embodiment, the antigen is Tyrosinase related protein 2. In another embodiment, the antigen is PSA (prostate-specific antigen). In another embodiment, the antigen is selected from HPV-E7, HPV-E6, NY-ESO-1, telomerase (TERT), SCCE, EGFR-III, survivin, baculoviral inhibitor of apoptosis repeat-containing 5 (BIRC5), WT-1, HIV-1 Gag, CEA, LMP-1, p53, PSMA, PSCA, Proteinase 3, Tyrosinase related protein 2, Muc1, PSA (prostate-specific antigen), or a combination thereof.

In one embodiment, a polypeptide expressed by the Listeria of the present invention may be a neuropeptide growth factor antagonist, which in one embodiment is [D-Arg1, D-Phe5, D-Trp7,9, Leu11] substance P, [Arg6, D-Trp7,9, NmePhe8]substance P(6-11). These and related embodiments embodiments are understood by one of skill in the art.

In another embodiment, the antigen is an infectious disease antigen. In one embodiment, the antigen is an auto antigen or a self-antigen.

In other embodiments, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, human papilloma virus antigens E1 and E2 from type HPV-16, -18, -31, -33, -35 or -45 human papilloma viruses, or a combination thereof.

In other embodiments, the antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough3 yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and lesteriosis.

The immune response induced by methods and compositions disclosed herein is, in another embodiment, a T cell response. In another embodiment, the immune response comprises a T cell response. In another embodiment, the response is a CD8+ T cell response. In another embodiment, the response comprises a CD8⁺ T cell response.

In one embodiment, a recombinant Listeria of the compositions and methods disclosed herein comprise an angiogenic polypeptide. In another embodiment, anti-angiogenic approaches to cancer therapy are very promising, and in one embodiment, one type of such anti-angiogenic therapy targets pericytes. In another embodiment, molecular targets on vascular endothelial cells and pericytes are important targets for antitumor therapies. In another embodiment, the platelet-derived growth factor receptor (PDGF-B/PDGFR-β) signaling is important to recruit pericytes to newly formed blood vessels. Thus, in one embodiment, angiogenic polypeptides disclosed herein inhibit molecules involved in pericyte signaling, which in one embodiment, is PDGFR-β.

In one embodiment, the compositions of the present invention comprise an angiogenic factor, or an immunogenic fragment thereof, where in one embodiment, the immunogenic fragment comprises one or more epitopes recognized by the host immune system. In one embodiment, an angiogenic factor is a molecule involved in the formation of new blood vessels. In one embodiment, the angiogenic factor is VEGFR2. In another embodiment, an angiogenic factor of the present invention is Angiogenin; Angiopoietin-1; Del-1; Fibroblast growth factors: acidic (aFGF) and basic (bFGF); Follistatin; Granulocyte colony-stimulating factor (G-CSF); Hepatocyte growth factor (HGF)/scatter factor (SF); Interleukin-8 (IL-8); Leptin; Midkine; Placental growth factor; Platelet-derived endothelial cell growth factor (PD-ECGF); Platelet-derived growth factor-BB (PDGF-BB); Pleiotrophin (PTN); Progranulin; Proliferin; survivin; Transforming growth factor-alpha (TGF-alpha); Transforming growth factor-beta (TGF-beta); Tumor necrosis factor-alpha (TNF-alpha); Vascular endothelial growth factor (VEGF)/vascular permeability factor (VPF). In another embodiment, an angiogenic factor is an angiogenic protein. In one embodiment, a growth factor is an angiogenic protein. In one embodiment, an angiogenic protein for use in the compositions and methods of the present invention is Fibroblast growth factors (FGF); VEGF; VEGFR and Neuropilin 1 (NRP-1); Angiopoietin 1 (Ang1) and Tie2; Platelet-derived growth factor (PDGF; BB-homodimer) and PDGFR; Transforming growth factor-beta (TGF-β), endoglin and TGF-β receptors; monocyte chemotactic protein-1 (MCP-1); Integrins αVβ3, αVβ5 and α5β1; VE-cadherin and CD31; ephrin; plasminogen activators; plasminogen activator inhibitor-1; Nitric oxide synthase (NOS) and COX-2; AC133; or Id1/Id3. In one embodiment, an angiogenic protein for use in the compositions and methods of the present invention is an angiopoietin, which in one embodiment, is Angiopoietin 1, Angiopoietin 3, Angiopoietin 4 or Angiopoietin 6. In one embodiment, endoglin is also known as CD105; EDG; HHT1; ORW; or ORW1. In one embodiment, endoglin is a TGFbeta co-receptor.

In one embodiment, cancer vaccines disclosed herein generate effector T cells that are able to infiltrate the tumor, destroy tumor cells and eradicate the disease. In one embodiment, naturally occurring tumor infiltrating lymphocytes (TILs) are associated with better prognosis in several tumors, such as colon, ovarian and melanoma. In colon cancer, tumors without signs of micrometastasis have an increased infiltration of immune cells and a Th1 expression profile, which correlate with an improved survival of patients. Moreover, the infiltration of the tumor by T cells has been associated with success of immunotherapeutic approaches in both pre-clinical and human trials. In one embodiment, the infiltration of lymphocytes into the tumor site is dependent on the up-regulation of adhesion molecules in the endothelial cells of the tumor vasculature, generally by proinflammatory cytokines, such as IFN-γ, TNF-α and IL-1. Several adhesion molecules have been implicated in the process of lymphocyte infiltration into tumors, including intercellular adhesion molecule 1 (ICAM-1), vascular endothelial cell adhesion molecule 1 (V-CAM-1), vascular adhesion protein 1 (VAP-1) and E-selectin. However, these cell-adhesion molecules are commonly down-regulated in the tumor vasculature. Thus, in one embodiment, cancer vaccines disclosed herein increase TILs, up-regulate adhesion molecules (in one embodiment, ICAM-1, V-CAM-1, VAP-1, E-selectin, or a combination thereof), up-regulate proinflammatory cytokines (in one embodiment, IFN-γ, TNF-α, IL-1, or a combination thereof), or a combination thereof.

In one embodiment, the compositions and methods disclosed herein provide anti-angiogenesis therapy, which in one embodiment, may improve immunotherapy strategies. In one embodiment, the compositions and methods disclosed herein circumvent endothelial cell anergy in vivo by up-regulating adhesion molecules in tumor vessels and enhancing leukocyte-vessel interactions, which increases the number of tumor infiltrating leukocytes, such as CD8⁺ T cells. Interestingly, enhanced anti-tumor protection correlates with an increased number of activated CD4⁺ and CD8⁺ tumor-infiltrating T cells and a pronounced decrease in the number of regulatory T cells in the tumor upon VEGF blockade.

In one embodiment, delivery of anti-angiogenic antigen simultaneously with a tumor-associated antigen to a host afflicted by a tumor as described herein, will have a synergistic effect in impacting tumor growth and a more potent therapeutic efficacy.

In another embodiment, targeting pericytes through vaccination will lead to cytotoxic T lymphocyte (CTL) infiltration, destruction of pericytes, blood vessel destabilization and vascular inflammation, which in another embodiment is associated with up-regulation of adhesion molecules in the endothelial cells that are important for lymphocyte adherence and transmigration, ultimately improving the ability of lymphocytes to infiltrate the tumor tissue. In another embodiment, concomitant delivery of a tumor-specific antigen generate lymphocytes able to invade the tumor site and kill tumor cells.

In one embodiment, the platelet-derived growth factor receptor (PDGF-B/PDGFR-β) signaling is important to recruit pericytes to newly formed blood vessels. In another embodiment, inhibition of VEGFR-2 and PDGFR-β concomitantly induces endothelial cell apoptosis and regression of tumor blood vessels, in one embodiment, approximately 40% of tumor blood vessels.

In another embodiment, said recombinant Listeria strain is an auxotrophic Listeria strain. In another embodiment, said auxotrophic Listeria strain is a dal/dat mutant. In another embodiment, the nucleic acid molecule is stably maintained in the recombinant bacterial strain in the absence of antibiotic selection.

In one embodiment the attenuated strain is Lm dal(−)dat(−) (Lmdd). In another embodiment, the attenuated strains is Lm dal(−)dat(−)AactA (LmddA). LmddA is based on a Listeria vaccine vector which is attenuated due to the deletion of virulence gene actA and retains the plasmid for a desired heterologous antigen or trunctated LLO expression in vivo and in vitro by complementation of dal gene.

In another embodiment the attenuated strain is Lmdda. In another embodiment, the attenuated strain is LmΔactA. In another embodiment, the attenuated strain is LmΔPrfA. In another embodiment, the attenuated strain is LmΔPlcB. In another embodiment, the attenuated strain is LmΔPlcA. In another embodiment, the strain is the double mutant or triple mutant of any of the above-mentioned strains. In another embodiment, this strain exerts a strong adjuvant effect which is an inherent property of Listeria-based vaccines. In another embodiment, this strain is constructed from the EGD Listeria backbone. In another embodiment, the strain used in the invention is a Listeria strain that expresses a non-hemolytic LLO. In yet another embodiment the Listeria strain is a prfA mutant, actA mutant, a plcB deletion mutant, or a double mutant lacking both plcA and plcB. All these Listeria strain are contemplated for use in the methods provided herein. Each possibility represents a separate embodiment of the present invention.

In one embodiment, translocation of Listeria to adjacent cells is inhibited by the deletion of the actA gene and/or the inlC gene, which are involved in the process, thereby resulting in unexpectedly high levels of attenuation with increased immunogenicity and utility as a vaccine backbone.

In one embodiment, the recombinant Listeria strain provided herein is attenuated. In another embodiment, the recombinant Listeria lacks the actA virulence gene. In another embodiment, the recombinant Listeria lacks the prfA virulence gene. In another embodiment, the recombinant Listeria lacks the inlB gene. In another embodiment, the recombinant Listeria lacks both, the actA and inlB genes. In another embodiment, the recombinant Listeria strain provided herein comprise an inactivating mutation of the endogenous actA gene. In another embodiment, the recombinant Listeria strain provided herein comprise an inactivating mutation of the endogenous inlB gene. In another embodiment, the recombinant Listeria strain provided herein comprise an inactivating mutation of the endogenous inlC gene. In another embodiment, the recombinant Listeria strain provided herein comprise an inactivating mutation of the endogenous actA and inlB genes. In another embodiment, the recombinant Listeria strain provided herein comprise an inactivating mutation of the endogenous actA and inlC genes. In another embodiment, the recombinant Listeria strain provided herein comprise an inactivating mutation of the endogenous actA, inlB, and inlC genes. In another embodiment, the recombinant Listeria strain provided herein comprise an inactivating mutation of the endogenous actA, inlB, and inlC genes. In another embodiment, the recombinant Listeria strain provided herein comprise an inactivating mutation of the endogenous actA, inlB, and inlC genes. In another embodiment, the recombinant Listeria strain provided herein comprise an inactivating mutation in any single gene or combination of the following genes: actA, dal, dat, inlB, inlC, prfA, plcA, plcB.

In one embodiment, auxotrophic mutants useful as vaccine vectors may be generated in a number of ways. In another embodiment, D-alanine auxotrophic mutants can be generated, in one embodiment, via the disruption of both the dal gene and the dat gene to generate an attenuated auxotrophic strain of Listeria which requires exogenously added D-alanine for growth. In another embodiment, the auxotrophy can be complemented via expression of the dal gene from a plasmid or episome.

In another embodiment, the recombinant Listeria strain provided herein comprise an inactivating mutation of the endogenous D-alanine racemase (Dal) gene. In another embodiment, the recombinant Listeria strain provided herein comprise an inactivating mutation of the endogenous D-amino acid transferase (Dat) gene. In another embodiment, the recombinant Listeria strain provided herein comprise an inactivating mutation of the endogenous Dal and Dat genes. In another embodiment, the recombinant Listeria strain provided herein comprise an inactivating mutation of the endogenous Dal/Dat and actA genes. In another embodiment, the recombinant Listeria strain provided herein comprise an inactivating mutation of the endogenous Dal/Dat/actA and inlB genes. In another embodiment, the recombinant Listeria strain provided herein comprises an inactivating mutation of the endogenous prfA gene. In another embodiment, the recombinant Listeria strain provided herein comprise an inactivating mutation of the endogenous Dal/Dat, actA and prfA genes. In another embodiment, the inactivating mutation is a deletion mutation. In another embodiment, the inactivating mutation is a truncation. In another embodiment, the inactivating mutation is a replacement or substitution mutation.

In one emobodiment, methods of making mutations are known in the art and are contemplated for use in the present invention.

In one embodiment, the generation of AA strains of Listeria deficient in D-alanine, for example, may be accomplished in a number of ways that are well known to those of skill in the art, including deletion mutagenesis, insertion mutagenesis, and mutagenesis which results in the generation of frameshift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols presented herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, disclosed herein, may be used as targets for mutagenesis of Listeria.

In one embodiment, said auxotrophic Listeria strain comprises an episomal expression vector comprising a metabolic enzyme that complements the auxotrophy of said auxotrophic Listeria strain. In another embodiment, the term “episomal” and grammatical equivalents thereof, refer to extrachromosomal DNA that can replicate autonomously in the cytoplasm of a host cell. In another embodiment, the episome is a plasmid. In another embodiment the episome is an expression vector.

In another embodiment, the plasmid is an integrative plasmid and comprises sequences that allow it to be integrated into the chromosome of the host.

In another embodiment, the construct provided herein is contained in the Listeria strain in an episomal fashion. In another embodiment, the foreign antigen is expressed from a vector harbored by the recombinant Listeria strain. In another embodiment, said episomal expression vector lacks an antibiotic resistance marker. In one embodiment, an antigen of the methods and compositions disclosed herein is genetically fused to an oligopeptide comprising a PEST sequence. In another embodiment, said endogenous polypeptide comprising a PEST sequence is LLO. In another embodiment, said endogenous polypeptide comprising a PEST sequence is ActA.

In another embodiment, the metabolic enzyme complements an endogenous metabolic gene that is lacking in the remainder of the chromosome of the recombinant bacterial strain. In one embodiment, the endogenous metabolic gene is mutated in the chromosome. In another embodiment, the endogenous metabolic gene is deleted from the chromosome. In another embodiment, said metabolic enzyme is an amino acid metabolism enzyme. In another embodiment, said metabolic enzyme catalyzes a formation of an amino acid used for a cell wall synthesis in said recombinant Listeria strain. In another embodiment, said metabolic enzyme is an alanine racemase enzyme. In another embodiment, said metabolic enzyme is a D-amino acid transferase enzyme.

In another embodiment, the metabolic enzyme catalyzes the formation of an amino acid (AA) used in cell wall synthesis. In another embodiment, the metabolic enzyme catalyzes synthesis of an AA used in cell wall synthesis. In another embodiment, the metabolic enzyme is involved in synthesis of an AA used in cell wall synthesis. In another embodiment, the AA is used in cell wall biogenesis.

In another embodiment, the metabolic enzyme is a synthetic enzyme for D-glutamic acid, a cell wall component.

In another embodiment, the metabolic enzyme is encoded by an alanine racemase gene (dal) gene. In another embodiment, the dal gene encodes alanine racemase, which catalyzes the reaction L-alanine [symbol] D-alanine.

The dal gene of methods and compositions of the methods and compositions disclosed herein is encoded, in another embodiment, by the sequence:

atggtgacaggctggcatcgtccaacatggattgaaatagaccgcgcagcaattcgcgaaaatataaaaaatgaacaa aataaactcccggaaagtgtcgacttatgggcagtagtcaaagctaatgcatatggtcacggaattatcgaagttgctaggacggcga aagaagctggagcaaaaggtttctgcgtagccattttagatgaggcactggctcttagagaagctggatttcaagatgactttattcttgt gcttggtgcaaccagaaaagaagatgctaatctggcagccaaaaaccacatttcacttactgatttagagaagattggctagagaatct aacgctagaagcaacacttcgaattcatttaaaagtagatagcggtatggggcgtctcggtattcgtacgactgaagaagcacggcga attgaagcaaccagtactaatgatcaccaattacaactggaaggtatttacacgcattttgcaacagccgaccagctagaaactagttatt ttgaacaacaattagctaagttccaaacgattttaacgagtttaaaaaaacgaccaacttatgttcatacagccaattcagctgcttcattgt tacagccacaaatcgggtttgatgcgattcgctttggtatttcgatgtatggattaactccctccacagaaatcaaaactagcttgccgttt gagcttaaacctgcacttgcactctataccgagatggttcatgtgaaagaacttgcaccaggcgatagcgttagctacggagcaacttat acagcaacagagcgagaatgggttgcgacattaccaattggctatgcggatggattgattcgtcattacagtggtttccatgttttagtag acggtgaaccagctccaatcattggtcgagtttgtatggatcaaaccatcataaaactaccacgtgaatttcaaactggttcaaaagtaac gataattggcaaagatcatggtaacacggtaacagcagatgatgccgctcaatatttagatacaattaattatgaggtaacttgtttgttaa atgagcgcatacctagaaaatacatccattag (SEQ ID No: 24 GenBank Accession No: AF038438). In another embodiment, the nucleotide encoding dal is homologous to SEQ ID No: 24. In another embodiment, the nucleotide encoding dal is a variant of SEQ ID No: 24. In another embodiment, the nucleotide encoding dal is a fragment of SEQ ID No: 24. In another embodiment, the dal protein is encoded by any other dal gene known in the art.

In another embodiment, the dal protein has the sequence:

MVTGWHRPTWIEIDRAAIRENIKNEQNKLPESVDLWAVVKANAYGHGIIEV ARTAKEAGAKGFCVAILDEALALREAGFQDDFILVLGATRKEDANLAAKNHISLTVF REDWLENLTLEATLRIHLKVDSGMGRLGIRTTEEARRIEATSTNDHQLQLEGIYTHFA TADQLETSYFEQQLAKFQTILTSLKKRPTYVHTANSAASLLQPQIGFDAIRFGISMYG LTPSTEIKTSLPFELKPALALYTEMVHVKELAPGDSVSYGATYTATEREWVATLPIGY ADGLIRHYSGFHVLVDGEPAPIIGRVCMDQTIIKLPREFQTGSKVTIIGKDHGNTVTA DDAAQYLDTINYEVTCLLNERIPRKYIH (SEQ ID No: 25; GenBank Accession No: AF038428). In another embodiment, the dal protein is homologous to SEQ ID No: 25. In another embodiment, the dal protein is a variant of SEQ ID No: 25. In another embodiment, the dal protein is an isomer of SEQ ID No: 25. In another embodiment, the dal protein is a fragment of SEQ ID No: 25. In another embodiment, the dal protein is a fragment of a homologue of SEQ ID No: 25. In another embodiment, the dal protein is a fragment of a variant of SEQ ID No: 25. In another embodiment, the dal protein is a fragment of an isomer of SEQ ID No: 25.

In another embodiment, the dal protein is any other Listeria dal protein known in the art. In another embodiment, the dal protein is any other gram-positive dal protein known in the art. In another embodiment, the dal protein is any other dal protein known in the art.

In another embodiment, the dal protein of methods and compositions disclosed herein retains its enzymatic activity. In another embodiment, the dal protein retains 90% of wild-type activity. In another embodiment, the dal protein retains 80% of wild-type activity. In another embodiment, the dal protein retains 70% of wild-type activity. In another embodiment, the dal protein retains 60% of wild-type activity. In another embodiment, the dal protein retains 50% of wild-type activity. In another embodiment, the dal protein retains 40% of wild-type activity. In another embodiment, the dal protein retains 30% of wild-type activity. In another embodiment, the dal protein retains 20% of wild-type activity. In another embodiment, the dal protein retains 10% of wild-type activity. In another embodiment, the dal protein retains 5% of wild-type activity.

In another embodiment, the metabolic enzyme is encoded by a D-amino acid aminotransferase gene (dat). D-glutamic acid synthesis is controlled in part by the dat gene, which is involved in the conversion of D-glu+pyr to alpha-ketoglutarate+D-ala, and the reverse reaction.

In another embodiment, a dat gene utilized in the present invention has the sequence set forth in GenBank Accession Number AF038439. In another embodiment, the dat gene is any another dat gene known in the art.

The dat gene of methods and compositions of the methods and compositions disclosed herein is encoded, in another embodiment, by the sequence:

atgaaagtattagtaaataaccatttagttgaaagagaagatgccacagttgacattgaagaccgcggatatcagtttggt gatggtgtatatgaagtagttcgtctatataatggaaaattctttacttataatgaacacattgatcgcttatatgctagtgcagcaaaaattg acttagttattccttattccaaagaagagctacgtgaattacttgaaaaattagttgccgaaaataatatcaatacagggaatgtctatttac aagtgactcgtggtgttcaaaacccacgtaatcatgtaatccctgatgatttccctctagaaggcgttttaacagcagcagctcgtgaagt acctagaaacgagcgtcaattcgttgaaggtggaacggcgattacagaagaagatgtgcgctggttacgctgtgatattaagagcttaa accttttaggaaatattctagcaaaaaataaagcacatcaacaaaatgctttggaagctattttacatcgcggggaacaagtaacagaat gttctgcttcaaacgtttctattattaaagatggtgtattatggacgcatgcggcagataacttaatcttaaatggtatcactcgtcaagttat cattgatgttgcgaaaaagaatggcattcctgttaaagaagcggatttcactttaacagaccttcgtgaagcggatgaagtgttcatttca agtacaactattgaaattacacctattacgcatattgacggagttcaagtagctgacggaaaacgtggaccaattacagcgcaacttcat caatattttgtagaagaaatcactcgtgcatgtggcgaattagagtttgcaaaataa (SEQ ID No: 26; GenBank Accession No: AF038439). In another embodiment, the nucleotide encoding dat is homologous to SEQ ID No: 26. In another embodiment, the nucleotide encoding dat is a variant of SEQ ID No: 26. In another embodiment, the nucleotide encoding dat is a fragment of SEQ ID No: 26. In another embodiment, the dat protein is encoded by any other dat gene known in the art.

In another embodiment, the dat protein has the sequence:

MKVLVNNHLVEREDATVDIEDRGYQFGDGVYEVVRLYNGKFFTYNEHIDR LYASAAKIDLVIPYSKEELRELLEKLVAENNINTGNVYLQVTRGVQNPRNHVIPDDFP LEGVLTAAAREVPRNERQFVEGGTAITEEDVRWLRCDIKSLNLLGNILAKNKAHQQ NALEAILHRGEQVTECSASNVSIIKDGVLWTHAADNLILNGITRQVIIDVAKKNGIPV KEADFTLTDLREADEVFISSTTIEITPITHIDGVQVADGKRGPITAQLHQYFVEEITRAC GELEFAK (SEQ ID No: 27; GenBank Accession No: AF038439). In another embodiment, the dat protein is homologous to SEQ ID No: 27. In another embodiment, the dat protein is a variant of SEQ ID No: 27. In another embodiment, the dat protein is an isomer of SEQ ID No: 27. In another embodiment, the dat protein is a fragment of SEQ ID No: 27. In another embodiment, the dat protein is a fragment of a homologue of SEQ ID No: 27. In another embodiment, the dat protein is a fragment of a variant of SEQ ID No: 27. In another embodiment, the dat protein is a fragment of an isomer of SEQ ID No: 27.

In another embodiment, the dat protein is any other Listeria dat protein known in the art. In another embodiment, the dat protein is any other gram-positive dat protein known in the art. In another embodiment, the dat protein is any other dat protein known in the art.

In another embodiment, the dat protein of methods and compositions of the methods and compositions disclosed herein retains its enzymatic activity. In another embodiment, the dat protein retains 90% of wild-type activity. In another embodiment, the dat protein retains 80% of wild-type activity. In another embodiment, the dat protein retains 70% of wild-type activity. In another embodiment, the dat protein retains 60% of wild-type activity. In another embodiment, the dat protein retains 50% of wild-type activity. In another embodiment, the dat protein retains 40% of wild-type activity. In another embodiment, the dat protein retains 30% of wild-type activity. In another embodiment, the dat protein retains 20% of wild-type activity. In another embodiment, the dat protein retains 10% of wild-type activity. In another embodiment, the dat protein retains 5% of wild-type activity.

In another embodiment, the metabolic enzyme is encoded by dga. D-glutamic acid synthesis is also controlled in part by the dga gene, and an auxotrophic mutant for D-glutamic acid synthesis will not grow in the absence of D-glutamic acid (Pucci et al, 1995, J Bacteriol. 177: 336-342). In another rembodiment, the recombinant Listeria is auxotrophic for D-glutamic acid. A further example includes a gene involved in the synthesis of diaminopimelic acid. Such synthesis genes encode beta-semialdehyde dehydrogenase, and when inactivated, renders a mutant auxotrophic for this synthesis pathway (Sizemore et al, 1995, Science 270: 299-302). In another embodiment, the dga protein is any other Listeria dga protein known in the art. In another embodiment, the dga protein is any other gram-positive dga protein known in the art.

In another embodiment, the metabolic enzyme is encoded by an alr (alanine racemase) gene. In another embodiment, the metabolic enzyme is any other enzyme known in the art that is involved in alanine synthesis. In another embodiment, the metabolic enzyme is any other enzyme known in the art that is involved in L-alanine synthesis. In another embodiment, the metabolic enzyme is any other enzyme known in the art that is involved in D-alanine synthesis. In another rembodiment, the recombinant Listeria is auxotrophic for D-alanine. Bacteria auxotrophic for alanine synthesis are well known in the art, and are described in, for example, E. coli (Strych et al, 2002, J. Bacteriol. 184:4321-4325), Corynebacterium glutamicum (Tauch et al, 2002, J. Biotechnol 99:79-91), and Listeria monocytogenes (Frankel et al, U.S. Patent 6,099,848)), Lactococcus species, and Lactobacillus species, (Bron et al, 2002, Appl Environ Microbiol, 68: 5663-70). In another embodiment, any D-alanine synthesis gene known in the art is inactivated.

In another embodiment, the metabolic enzyme is an amino acid aminotransferase.

In another embodiment, the metabolic enzyme is encoded by serC, a phosphoserine aminotransferase. In another embodiment, the metabolic enzyme is encoded by asd (aspartate beta-semialdehyde dehydrogenase), involved in synthesis of the cell wall constituent diaminopimelic acid. In another embodiment, the metabolic enzyme is encoded by gsaB-glutamate-1-semialdehyde aminotransferase, which catalyzes the formation of 5-aminolevulinate from (S)-4-amino-5-oxopentanoate. In another embodiment, the metabolic enzyme is encoded by HemL, which catalyzes the formation of 5-aminolevulinate from (S)-4-amino-5-oxopentanoate. In another embodiment, the metabolic enzyme is encoded by aspB, an aspartate aminotransferase that catalyzes the formation of oxalozcetate and L-glutamate from L-aspartate and 2-oxoglutarate. In another embodiment, the metabolic enzyme is encoded by argF-1, involved in arginine biosynthesis. In another embodiment, the metabolic enzyme is encoded by aroE, involved in amino acid biosynthesis. In another embodiment, the metabolic enzyme is encoded by aroB, involved in 3-dehydroquinate biosynthesis. In another embodiment, the metabolic enzyme is encoded by aroD, involved in amino acid biosynthesis. In another embodiment, the metabolic enzyme is encoded by aroC, involved in amino acid biosynthesis. In another embodiment, the metabolic enzyme is encoded by hisB, involved in histidine biosynthesis. In another embodiment, the metabolic enzyme is encoded by hisD, involved in histidine biosynthesis. In another embodiment, the metabolic enzyme is encoded by hisG, involved in histidine biosynthesis. In another embodiment, the metabolic enzyme is encoded by metX, involved in methionine biosynthesis. In another embodiment, the metabolic enzyme is encoded by proB, involved in proline biosynthesis. In another embodiment, the metabolic enzyme is encoded by argR, involved in arginine biosynthesis. In another embodiment, the metabolic enzyme is encoded by argJ, involved in arginine biosynthesis. In another embodiment, the metabolic enzyme is encoded by thiI, involved in thiamine biosynthesis. In another embodiment, the metabolic enzyme is encoded by LMOf2365_1652, involved in tryptophan biosynthesis. In another embodiment, the metabolic enzyme is encoded by aroA, involved in tryptophan biosynthesis. In another embodiment, the metabolic enzyme is encoded by ilvD, involved in valine and isoleucine biosynthesis. In another embodiment, the metabolic enzyme is encoded by ilvC, involved in valine and isoleucine biosynthesis. In another embodiment, the metabolic enzyme is encoded by leuA, involved in leucine biosynthesis. In another embodiment, the metabolic enzyme is encoded by dapF, involved in lysine biosynthesis. In another embodiment, the metabolic enzyme is encoded by thrB, involved in threonine biosynthesis (all GenBank Accession No. NC_002973).

In another embodiment, the metabolic enzyme is a tRNA synthetase. In another embodiment, the metabolic enzyme is encoded by the trpS gene, encoding tryptophanyltRNA synthetase. In another embodiment, the metabolic enzyme is any other tRNA synthetase known in the art.

In another embodiment, a recombinant Listeria strain disclosed herein has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the passaging attenuates the strain, or in another embodiment, makes the strain less virulent. Methods for passaging a recombinant Listeria strain through an animal host are well known in the art, and are described, for example, in United States Patent Application Serial No. 10/541,614.

The recombinant Listeria strain of the methods and compositions disclosed herein is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art. Each possibility represents a separate embodiment disclosed herein. In another embodiment, the sequences of Listeria proteins for use in the methods and compositions disclosed herein are from any of the above-described strains.

In one embodiment, a Listeria monocytogenes strain disclosed herein is the EGD strain, the 10403S strain, the NICPBP 54002 strain, the S3 strain, the NCTC 5348 strain, the NICPBP 54006 strain, the M7 strain, the S19 strain, or another strain of Listeria monocytogenes which is known in the art.

In another embodiment, the recombinant Listeria strain is a vaccine strain, which in one embodiment, is a bacterial vaccine strain.

In another embodiment, the recombinant Listeria strain utilized in methods of the present invention has been stored in a frozen cell bank. In another embodiment, the recombinant Listeria strain has been stored in a lyophilized cell bank.

In another embodiment, the cell bank of methods and compositions of the present invention is a master cell bank. In another embodiment, the cell bank is a working cell bank. In another embodiment, the cell bank is Good Manufacturing Practice (GMP) cell bank. In another embodiment, the cell bank is intended for production of clinical-grade material. In another embodiment, the cell bank conforms to regulatory practices for human use. In another embodiment, the cell bank is any other type of cell bank known in the art.

“Good Manufacturing Practices” are defined, in another embodiment, by (21 CFR 210-211) of the United States Code of Federal Regulations. In another embodiment, “Good Manufacturing Practices” are defined by other standards for production of clinical-grade material or for human consumption; e.g. standards of a country other than the United States.

In another embodiment, a recombinant Listeria strain utilized in methods of the present invention is from a batch of vaccine doses.

In another embodiment, a recombinant Listeria strain utilized in methods of the present invention is from a frozen stock produced by a method disclosed herein.

In another embodiment, a recombinant Listeria strain utilized in methods of the present invention is from a lyophilized stock produced by a method disclosed herein.

In another embodiment, a cell bank, frozen stock, or batch of vaccine doses of the present invention exhibits viability upon thawing of greater than 90%. In another embodiment, the thawing follows storage for cryopreservation or frozen storage for 24 hours. In another embodiment, the storage is for 2 days. In another embodiment, the storage is for 3 days. In another embodiment, the storage is for 4 days. In another embodiment, the storage is for 1 week. In another embodiment, the storage is for 2 weeks. In another embodiment, the storage is for 3 weeks. In another embodiment, the storage is for 1 month. In another embodiment, the storage is for 2 months. In another embodiment, the storage is for 3 months. In another embodiment, the storage is for 5 months. In another embodiment, the storage is for 6 months. In another embodiment, the storage is for 9 months. In another embodiment, the storage is for 1 year.

In another embodiment, a cell bank, frozen stock, or batch of vaccine doses of the present invention is cryopreserved by a method that comprises growing a culture of the Listeria strain in a nutrient media, freezing the culture in a solution comprising glycerol, and storing the Listeria strain at below −20 degrees Celsius. In another embodiment, the temperature is about −70 degrees Celsius. In another embodiment, the temperature is about ⁻70-⁻80 degrees Celsius.

In another embodiment of methods and compositions of the present invention, the culture (e.g. the culture of a Listeria vaccine strain that is used to produce a batch of Listeria vaccine doses) is inoculated from a cell bank. In another embodiment, the culture is inoculated from a frozen stock. In another embodiment, the culture is inoculated from a starter culture. In another embodiment, the culture is inoculated from a colony. In another embodiment, the culture is inoculated at mid-log growth phase. In another embodiment, the culture is inoculated at approximately mid-log growth phase. In another embodiment, the culture is inoculated at another growth phase.

In another embodiment of methods and compositions of the present invention, the solution used for freezing contains glycerol in an amount of 2-20%. In another embodiment, the amount is 2%. In another embodiment, the amount is 20%. In another embodiment, the amount is 1%. In another embodiment, the amount is 1.5%. In another embodiment, the amount is 3%. In another embodiment, the amount is 4%. In another embodiment, the amount is 5%. In another embodiment, the amount is 2%. In another embodiment, the amount is 2%. In another embodiment, the amount is 7%. In another embodiment, the amount is 9%. In another embodiment, the amount is 10%. In another embodiment, the amount is 12%. In another embodiment, the amount is 14%. In another embodiment, the amount is 16%. In another embodiment, the amount is 18%. In another embodiment, the amount is 222%. In another embodiment, the amount is 25%. In another embodiment, the amount is 30%. In another embodiment, the amount is 35%. In another embodiment, the amount is 40%.

In another embodiment, the solution used for freezing contains another colligative additive or additive with anti-freeze properties, in place of glycerol. In another embodiment, the solution used for freezing contains another colligative additive or additive with anti-freeze properties, in addition to glycerol. In another embodiment, the additive is mannitol. In another embodiment, the additive is DMSO. In another embodiment, the additive is sucrose. In another embodiment, the additive is any other colligative additive or additive with anti-freeze properties that is known in the art.

In one embodiment, a vaccine is a composition which elicits an immune response to an antigen or polypeptide in the composition as a result of exposure to the composition. In another embodiment, the vaccine additionally comprises an adjuvant, cytokine, chemokine, or combination thereof. In another embodiment, the vaccine or composition additionally comprises antigen presenting cells (APCs), which in one embodiment are autologous, while in another embodiment, they are allogeneic to the subject.

In one embodiment, a “vaccine” is a composition which elicits an immune response in a host to an antigen or polypeptide in the composition as a result of exposure to the composition. In one embodiment, the immune response is to a particular antigen or to a particular epitope on the antigen. In one embodiment, the vaccine may be a peptide vaccine, in another embodiment, a DNA vaccine. In another embodiment, the vaccine may be contained within and, in another embodiment, delivered by, a cell, which in one embodiment is a bacterial cell, which in one embodiment, is a Listeria. In one embodiment, a vaccine may prevent a subject from contracting or developing a disease or condition, wherein in another embodiment, a vaccine may be therapeutic to a subject having a disease or condition. In one embodiment, a vaccine of the present invention comprises a composition of the present invention and an adjuvant, cytokine, chemokine, or combination thereof.

In another embodiment, the present invention provides an immunogenic composition comprising a recombinant Listeria of the present invention. In another embodiment, the immunogenic composition of methods and compositions of the present invention comprises a recombinant vaccine vector of the present invention. In another embodiment, the immunogenic composition comprises a plasmid of the present invention. In another embodiment, the immunogenic composition comprises an adjuvant. In one embodiment, a vector of the present invention may be administered as part of a vaccine composition.

In another embodiment, a vaccine of the present invention is delivered with an adjuvant. In one embodiment, the adjuvant favors a predominantly Th1-mediated immune response. In another embodiment, the adjuvant favors a Th1-type immune response. In another embodiment, the adjuvant favors a Th1-mediated immune response. In another embodiment, the adjuvant favors a cell-mediated immune response over an antibody-mediated response. In another embodiment, the adjuvant is any other type of adjuvant known in the art. In another embodiment, the immunogenic composition induces the formation of a T cell immune response against the target protein.

In another embodiment, the adjuvant is MPL. In another embodiment, the adjuvant is QS21. In another embodiment, the adjuvant is a TLR agonist. In another embodiment, the adjuvant is a TLR4 agonist. In another embodiment, the adjuvant is a TLR9 agonist. In another embodiment, the adjuvant is Resiquimod®. In another embodiment, the adjuvant is imiquimod. In another embodiment, the adjuvant is a CpG oligonucleotide. In another embodiment, the adjuvant is a cytokine or a nucleic acid encoding same. In another embodiment, the adjuvant is a chemokine or a nucleic acid encoding same. In another embodiment, the adjuvant is IL-12 or a nucleic acid encoding same. In another embodiment, the adjuvant is IL-6 or a nucleic acid encoding same. In another embodiment, the adjuvant is a lipopolysaccharide. In another embodiment, the adjuvant is as described in Fundamental Immunology, 5th ed (August 2003): William E. Paul (Editor); Lippincott Williams & Wilkins Publishers; Chapter 43: Vaccines, GJV Nossal, which is hereby incorporated by reference. In another embodiment, the adjuvant is any other adjuvant known in the art. Each possibility represents a separate embodiment of the methods and compositions disclosed herein.

In one embodiment, provided herein is a method of inducing an immune response to an antigen in a subject comprising administering a recombinant Listeria strain to said subject. In one embodiment, provided herein is a method of inducing an anti-angiogenic immune response to an antigen in a subject comprising administering a recombinant Listeria strain to said subject. In another embodiment, said recombinant Listeria strain comprises a first and second nucleic acid molecule. In another embodiment, each said nucleic acid molecule encodes a heterologous antigen. In yet another embodiment, said first nucleic acid molecule is operably integrated into the Listeria genome as an open reading frame with an endogenous polypeptide comprising a PEST sequence.

In one embodiment, provided herein is a method of treating, suppressing, or inhibiting at least one cancer in a subject comprising administering a recombinant Listeria strain to said subject. In another embodiment, said recombinant Listeria strain comprises a first and second nucleic acid molecule. In another embodiment, each said nucleic acid molecule encoding a heterologous antigen. In yet another embodiment, said first nucleic acid molecule is operably integrated into the Listeria genome as an open reading frame with a nucleic acid sequence encoding an endogenous polypeptide comprising a PEST sequence. In another embodiment, at least one of said antigens is expressed by at least one cell of said cancer cells.

In one embodiment, provided herein is a method of delaying the onset to a cancer in a subject comprising administering a recombinant Listeria strain to said subject. In another embodiment, provided herein is a method of delaying the progression to a cancer in a subject comprising administering a recombinant Listeria strain to said subject. In another embodiment, provided herein is a method of extending the remission to a cancer in a subject comprising administering a recombinant Listeria strain to said subject. In another embodiment, provided herein is a method of decreasing the size of an existing tumor in a subject comprising administering a recombinant Listeria strain to said subject. In another embodiment, provided herein is a method of preventing the growth of an existing tumor in a subject comprising administering a recombinant Listeria strain to said subject. In another embodiment, provided herein is a method of preventing the growth of new or additional tumors in a subject comprising administering a recombinant Listeria strain to said subject.

In one embodiment, cancer or tumors may be prevented in specific populations known to be susceptible to a particular cancer or tumor. In one embodiment, such susceptibilty may be due to environmental factors, such as smoking, which in one embodiment, may cause a population to be subject to lung cancer, while in another embodiment, such susceptbility may be due to genetic factors, for example a population with BRCA1/2 mutations may be susceptible, in one embodiment, to breast cancer, and in another embodiment, to ovarian cancer. In another embodiment, one or more mutations on chromosome 8q24, chromosome 17q12, and chromosome 17q24.3 may increase susceptibility to prostate cancer, as is known in the art. Other genetic and environmental factors contributing to cancer susceptibility are known in the art.

In one embodiment, the recombinant Listeria strain is administered to the subject at a dose of 1×10⁶-1×10⁷ CFU. In another embodiment, the recombinant Listeria strain is administered to the subject at a dose of 1×10⁷-1×10⁸ CFU. In another embodiment, the recombinant Listeria strain is administered to the subject at a dose of 1×10⁸-3.31×10¹⁰ CFU. In another embodiment, the recombinant Listeria strain is administered to the subject at a dose of 1×10⁹-3.31×10¹⁰ CFU. In another embodiment, the dose is 5-500×10⁸ CFU. In another embodiment, the dose is 7-500×10⁸ CFU. In another embodiment, the dose is 10-500×10⁸ CFU. In another embodiment, the dose is 20-500×10⁸ CFU. In another embodiment, the dose is 30-500×10⁸ CFU. In another embodiment, the dose is 50-500×10⁸ CFU. In another embodiment, the dose is 70-500×10⁸ CFU. In another embodiment, the dose is 100-500×10⁸ CFU. In another embodiment, the dose is 150-500×10⁸ CFU. In another embodiment, the dose is 5-300×10⁸ CFU. In another embodiment, the dose is 5-200×10⁸ CFU. In another embodiment, the dose is 5-15×10⁸ CFU. In another embodiment, the dose is 5-100×10⁸ CFU. In another embodiment, the dose is 5-70×10⁸ CFU. In another embodiment, the dose is 5-50×10⁸ CFU. In another embodiment, the dose is 5-30×10⁸ CFU. In another embodiment, the dose is 5-20×10⁸ CFU. In another embodiment, the dose is 1-30×10⁹ CFU. In another embodiment, the dose is 1-20×10⁹CFU. In another embodiment, the dose is 2-30×10⁹ CFU. In another embodiment, the dose is 1-10×10⁹ CFU. In another embodiment, the dose is 2-10×10⁹ CFU. In another embodiment, the dose is 3-10×10⁹ CFU. In another embodiment, the dose is 2-7×10⁹ CFU. In another embodiment, the dose is 2-5×10⁹ CFU. In another embodiment, the dose is 3-5×10⁹ CFU.

In another embodiment, the dose is 1×10⁷ organisms. In another embodiment, the dose is 1.5×10⁷ organisms. In another embodiment, the dose is 2×10⁸ organisms. In another embodiment, the dose is 3×10⁷ organisms. In another embodiment, the dose is 4×10⁷ organisms. In another embodiment, the dose is 5×10⁷ organisms. In another embodiment, the dose is 6×10⁷ organisms. In another embodiment, the dose is 7×10⁷ organisms. In another embodiment, the dose is 8×10⁷ organisms. In another embodiment, the dose is 10×10⁷ organisms. In another embodiment, the dose is 1.5×10⁸ organisms. In another embodiment, the dose is 2×10⁸ organisms. In another embodiment, the dose is 2.5×10⁸ organisms. In another embodiment, the dose is 3×10⁸ organisms. In another embodiment, the dose is 3.3×10⁸ organisms. In another embodiment, the dose is 4×10⁸ organisms. In another embodiment, the dose is 5×10⁸ organisms.

In another embodiment, the dose is 1×10⁹ organisms. In another embodiment, the dose is 1.5×10⁹ organisms. In another embodiment, the dose is 2×10⁹ organisms. In another embodiment, the dose is 3×10⁹ organisms. In another embodiment, the dose is 4×10⁹ organisms. In another embodiment, the dose is 5×10⁹ organisms. In another embodiment, the dose is 6×10⁹ organisms. In another embodiment, the dose is 7×10⁹ organisms. In another embodiment, the dose is 8×10⁹ organisms. In another embodiment, the dose is 10×10⁹ organisms. In another embodiment, the dose is 1.5×10¹⁰ organisms. In another embodiment, the dose is 2×10¹⁰ organisms. In another embodiment, the dose is 2.5×10¹⁰ organisms. In another embodiment, the dose is 3×10¹⁰ organisms. In another embodiment, the dose is 3.3×10¹⁰ organisms. In another embodiment, the dose is 4×10¹⁰ organisms. In another embodiment, the dose is 5×10¹⁰ organisms.

In another embodiment, a method of the present invention further comprises boosting the subject with a recombinant Listeria strain provided herein. In another embodiment, a method of the present invention comprises the step of administering a booster dose of vaccine comprising the recombinant Listeria strain provided herein.

It will be appreciated by the skilled artisan that the term “Boosting” may encompass administering an additional vaccine or immunogenic composition or recombinant Listeria strain dose to a subject. In another embodiment of methods of the present invention, 2 boosts (or a total of 3 inoculations) are administered. In another embodiment, 3 boosts are administered. In another embodiment, 4 boosts are administered. In another embodiment, 5 boosts are administered. In another embodiment, 6 boosts are administered. In another embodiment, more than 6 boosts are administered.

In another embodiment, the recombinant Listeria strain used in the booster inoculation is the same as the strain used in the initial “priming” inoculation. In another embodiment, the booster strain is different from the priming strain. In another embodiment, the booster dose is an alternate form of said immunogenic composition. In another embodiment, the same doses are used in the priming and boosting inoculations. In another embodiment, a larger dose is used in the booster. In another embodiment, a smaller dose is used in the booster. In another embodiment, the methods of the present invention further comprise the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost vaccine is experimentally determined by the skilled artisan. In another embodiment, the period between a prime and a boost vaccine is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost vaccine is administered 8-10 weeks after the prime vaccine.

In another embodiment, a method of the present invention further comprises boosting the human subject with a recombinant Listeria strain provided herein. In another embodiment, a method of the present invention comprises the step of administering a booster dose of an immunogenic composition comprising the recombinant Listeria strain provided herein. In another embodiment, the booster dose is an alternate form of said immunogenic composition. In another embodiment, the methods of the present invention further comprise the step of administering to the subject a booster immunogenic composition. In one embodiment, the booster dose follows a single priming dose of said immunogenic composition. In another embodiment, a single booster dose is administered after the priming dose. In another embodiment, two booster doses are administered after the priming dose. In another embodiment, three booster doses are administered after the priming dose. In one embodiment, the period between a prime and a boost dose of an immunogenic composition comprising the recombinant Listeria provided herein is experimentally determined by the skilled artisan. In another embodiment, the dose is experimentally determined by a skilled artisan. In another embodiment, the period between a prime and a boost dose is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost dose is administered 8-10 weeks after the prime dose of the immunogenic composition.

Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Vaccine 20:1226-31 (2002); Tanghe, A., Infect. Immun. 69:3041-7 (2001). Providing antigen in different forms in the prime and the boost injections appears to maximize the immune response to the antigen. DNA vaccine priming followed by boosting with protein in adjuvant or by viral vector delivery of DNA encoding antigen appears to be the most effective way of improving antigen specific antibody and CD4+ T-cell responses or CD8+ T-cell responses respectively. Shiver J. W. et al., Nature 415: 331-5 (2002); Gilbert, S. C. et al., Vaccine 20:1039-45 (2002); Billaut-Mulot, O. et al., Vaccine 19:95-102 (2000); Sin, J. I. et al., DNA Cell Biol. 18:771-9 (1999). Recent data from monkey vaccination studies suggests that adding CRL1005 poloxamer (12 kDa, 5% POE), to DNA encoding the HIV gag antigen enhances T-cell responses when monkeys are vaccinated with an HIV gag DNA prime followed by a boost with an adenoviral vector expressing HIV gag (Ad5-gag). The cellular immune responses for a DNA/poloxamer prime followed by an Ad5-gag boost were greater than the responses induced with a DNA (without poloxamer) prime followed by Ad5-gag boost or for Ad5-gag only. Shiver, J. W. et al. Nature 415:331-5 (2002). U.S. Patent Appl. Publication No. US 2002/0165172 Al describes simultaneous administration of a vector construct encoding an immunogenic portion of an antigen and a protein comprising the immunogenic portion of an antigen such that an immune response is generated. The document is limited to hepatitis B antigens and HIV antigens. Moreover, U.S. Pat. No. 6,500,432 is directed to methods of enhancing an immune response of nucleic acid vaccination by simultaneous administration of a polynucleotide and polypeptide of interest. According to the patent, simultaneous administration means administration of the polynucleotide and the polypeptide during the same immune response, preferably within 0-10 or 3-7 days of each other. The antigens contemplated by the patent include, among others, those of Hepatitis (all forms), HSV, HIV, CMV, EBV, RSV, VZV, HPV, polio, influenza, parasites (e.g., from the genus Plasmodium), and pathogenic bacteria (including but not limited to M. tuberculosis, M. leprae, Chlamydia, Shigella, B. burgdorferi, enterotoxigenic E. coli, S. typhosa, H. pylori, V. cholerae, B. pertussis, etc.). All of the above references are herein incorporated by reference in their entireties.

In one embodiment, the nucleic acid molecule encodes a survivin and the method is for treating, inhibiting or suppressing lymphoma. In another embodiment, the nucleic acid molecule encodes survivin and the method is for treating, inhibiting or suppressing breast cancer or any other type of cancer provided herein. In another embodiment, the nucleic acid molecule encodes survivin and the method is treating, inhibiting, or suppressing metastasis of lymphoma, which in one embodiment, comprises metastasis to bone, and in another embodiment, comprises metastasis to other organs.

In one embodiment, the lymphoma is a B-cell lymphoma, a diffuse large B-cell lymphoma (DLBCL), a hodgkin lymphoma (HL), a non-hodgkin lymphoma (NHL), or a combination thereof.

The cancer that is the target of methods and compositions disclosed herein is, in another embodiment, a melanoma. In another embodiment, the cancer is a sarcoma. In another embodiment, the cancer is a carcinoma. In another embodiment, the cancer is a mesothelioma (e.g. malignant mesothelioma). In another embodiment, the cancer is a glioma. In another embodiment, the cancer is a germ cell tumor. In another embodiment, the cancer is a choriocarcinoma.

In another embodiment, the cancer is pancreatic cancer. In another embodiment, the cancer is ovarian cancer. In another embodiment, the cancer is lymphoma. In another embodiment, the cancer is gastric cancer. In another embodiment, the cancer is a carcinomatous lesion of the pancreas. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non small-cell lung carcinoma. In another embodiment, the cancer is an endometrial carcinoma. In another embodiment, the cancer is a bladder cancer. In another embodiment, the cancer is a head and neck cancer. In another embodiment, the cancer is anal cancer. In another embodiment, the cancer is esophageal cancer. In another embodiment, the cancer is gastric cancer. In another embodiment, the cancer is a prostate carcinoma.

In another embodiment, the cancer is a non-small cell lung cancer (NSCLC). In another embodiment, the cancer is a colon cancer. In another embodiment, the cancer is a lung cancer. In another embodiment, the cancer is an ovarian cancer. In another embodiment, the cancer is a uterine cancer. In another embodiment, the cancer is a thyroid cancer. In another embodiment, the cancer is a hepatocellular carcinoma. In another embodiment, the cancer is a thyroid cancer. In another embodiment, the cancer is a liver cancer. In another embodiment, the cancer is a renal cancer. In another embodiment, the cancer is a kaposis. In another embodiment, the cancer is a sarcoma. In another embodiment, the cancer is another carcinoma or sarcoma.

In one embodiment, the compositions and methods disclosed herein can be used to treat solid tumors related to or resulting from any of the cancers as described herein. In another embodiment, the tumor is a Wilms' tumor. In another embodiment, the tumor is a desmoplastic small round cell tumor.

In another embodiment, the vaccine is tested in human subjects, and efficacy is monitored using methods well known in the art, e.g. directly measuring CD4⁺ and CD8⁺ T cell responses, or measuring disease progression, e.g. by determining the number or size of tumor metastases, or monitoring disease symptoms (cough, chest pain, weight loss, etc). Methods for assessing the efficacy of a prostate cancer vaccine in human subjects are well known in the art, and are described, for example, in Uenaka A et al (T cell immunomonitoring and tumor responses in patients immunized with a complex of cholesterol-bearing hydrophobized pullulan (CHP) and NY-ESO-1 protein. Cancer Immun 2007 Apr 19;7:9) and Thomas-Kaskel AK et al (Vaccination of advanced prostate cancer patients with PSCA and PSA peptide-loaded dendritic cells induces DTH responses that correlate with superior overall survival. Int J Cancer. 2006 Nov 15;119(10):2428-34).

In one embodiment, provided herein is a recombinant Listeria strain comprising a nucleic acid molecule operably integrated into the Listeria genome. In another embodiment said nucleic acid molecule encodes (a) an endogenous polypeptide comprising a PEST sequence and (b) a polypeptide comprising an antigen in an open reading frame.

In one embodiment, provided herein is a method of treating, suppressing, or inhibiting at least one tumor in a subject, comprising administering a recombinant Listeria strain to said subject. In another embodiment, said recombinant Listeria strain comprises a first and second nucleic acid molecule. In another embodiment, each of said nucleic acid molecule encodes a heterologous antigen. In another embodiment, said first nucleic acid molecule is operably integrated into the Listeria genome as an open reading frame with a native polypeptide comprising a PEST sequence and wherein said antigen is expressed by at least one cell of said tumor. In another embodiment, both the first and second nucleic acid molecules are episomally expressed.

In one embodiment, the term “antigen” refers to a substance that when placed in contact with an organism, results in a detectable immune response from the organism. An antigen may be a lipid, peptide, protein, carbohydrate, nucleic acid, or combinations and variations thereof.

In one embodiment, “variant” refers to an amino acid or nucleic acid sequence (or in other embodiments, an organism or tissue) that is different from the majority of the population but is still sufficiently similar to the common mode to be considered to be one of them, for example splice variants.

In one embodiment, “isoform” refers to a version of a molecule, for example, a protein, with only slight differences compared to another isoform, or version, of the same protein. In one embodiment, isoforms may be produced from different but related genes, or in another embodiment, may arise from the same gene by alternative splicing. In another embodiment, isoforms are caused by single nucleotide polymorphisms.

In one embodiment, “fragment” refers to a protein or polypeptide that is shorter or comprises fewer amino acids than the full length protein or polypeptide. In another embodiment, fragment refers to a nucleic acid that is shorter or comprises fewer nucleotides than the full length nucleic acid. In another embodiment, the fragment is an N-terminal fragment. In another embodiment, the fragment is a C-terminal fragment. In one embodiment, the fragment is an intrasequential section of the protein, peptide, or nucleic acid. In one embodiment, the fragment is a functional fragment. In another embodiment, the fragment is an immunogenic fragment. In one embodiment, a fragment has 10-20 nucleic or amino acids, while in another embodiment, a fragment has more than 5 nucleic or amino acids, while in another embodiment, a fragment has 100-200 nucleic or amino acids, while in another embodiment, a fragment has 100-500 nucleic or amino acids, while in another embodiment, a fragment has 50-200 nucleic or amino acids, while in another embodiment, a fragment has 10-250 nucleic or amino acids.

In one embodiment, “immunogenicity” or “immunogenic” is used herein to refer to the innate ability of a protein, peptide, nucleic acid, antigen or organism to elicit an immune response in an animal when the protein, peptide, nucleic acid, antigen or organism is administered to the animal. Thus, “enhancing the immunogenicity” in one embodiment, refers to increasing the ability of a protein, peptide, nucleic acid, antigen or organism to elicit an immune response in an animal when the protein, peptide, nucleic acid, antigen or organism is administered to an animal. The increased ability of a protein, peptide, nucleic acid, antigen or organism to elicit an immune response can be measured by, in one embodiment, a greater number of antibodies to a protein, peptide, nucleic acid, antigen or organism, a greater diversity of antibodies to an antigen or organism, a greater number of T-cells specific for a protein, peptide, nucleic acid, antigen or organism, a greater cytotoxic or helper T-cell response to a protein, peptide, nucleic acid, antigen or organism, and the like.

In one embodiment, a “homologue” refers to a nucleic acid or amino acid sequence which shares a certain percentage of sequence identity with a particular nucleic acid or amino acid sequence. In one embodiment, a sequence useful in the composition and methods disclosed herein may be a homologue of a particular LLO sequence or N-terminal fragment thereof, ActA sequence or N-terminal fragment thereof, or PEST sequence described herein or known in the art. In one embodiment, such a homolog maintains In another embodiment, a sequence useful in the composition and methods disclosed herein may be a homologue of an antigenic polypeptide, which in one embodiment, is survivin or a fragment thereof. In one embodiment, a homolog of a polypeptide and, in one embodiment, the nucleic acid encoding such a homolog, of the present invention maintains the functional characteristics of the parent polypeptide. For example, in one embodiment, a homolog of an antigenic polypeptide of the present invention maintains the antigenic characteristic of the parent polypeptide. In another embodiment, a sequence useful in the composition and methods disclosed herein may be a homologue of any sequence described herein. In one embodiment, a homologue shares at least 68% identity with a particular sequence. In another embodiment, a homologue shares at least 70% identity with a particular sequence. In another embodiment, a homologue shares at least 72% identity with a particular sequence. In another embodiment, a homologue shares at least 75% identity with a particular sequence. In another embodiment, a homologue shares at least 78% identity with a particular sequence. In another embodiment, a homologue shares at least 80% identity with a particular sequence. In another embodiment, a homologue shares at least 82% identity with a particular sequence. In another embodiment, a homologue shares at least 83% identity with a particular sequence. In another embodiment, a homologue shares at least 85% identity with a particular sequence. In another embodiment, a homologue shares at least 87% identity with a particular sequence. In another embodiment, a homologue shares at least 88% identity with a particular sequence. In another embodiment, a homologue shares at least 90% identity with a particular sequence. In another embodiment, a homologue shares at least 92% identity with a particular sequence. In another embodiment, a homologue shares at least 93% identity with a particular sequence. In another embodiment, a homologue shares at least 95% identity with a particular sequence. In another embodiment, a homologue shares at least 96% identity with a particular sequence. In another embodiment, a homologue shares at least 97% identity with a particular sequence. In another embodiment, a homologue shares at least 98% identity with a particular sequence. In another embodiment, a homologue shares at least 99% identity with a particular sequence. In another embodiment, a homologue shares 100% identity with a particular sequence.

In one embodiment, it is to be understood that a homolog of any of the sequences disclosed herein and/or as described herein is considered to be a part of the invention.

In one embodiment, “functional” within the meaning of the invention, is used herein to refer to the innate ability of a protein, peptide, nucleic acid, fragment or a variant thereof to exhibit a biological activity or function. In one embodiment, such a biological function is its binding property to an interaction partner, e.g., a membrane-associated receptor, and in another embodiment, its trimerization property. In the case of functional fragments and the functional variants of the invention, these biological functions may in fact be changed, e.g., with respect to their specificity or selectivity, but with retention of the basic biological function.

In one embodiment, “treating” refers to both therapeutic treatment and prophylactic or preventative measures, wherein the object is to prevent or lessen the targeted pathologic condition or disorder as described herein. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” or “impeding” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, symptoms are primary, while in another embodiment, symptoms are secondary. In one embodiment, “primary” refers to a symptom that is a direct result of a particular disease or disorder, while in one embodiment, “secondary” refers to a symptom that is derived from or consequent to a primary cause. In one embodiment, the compounds for use in the present invention treat primary or secondary symptoms or secondary complications. In another embodiment, “symptoms” may be any manifestation of a disease or pathological condition.

In one embodiment, the compositions for use in the methods disclosed herein are administered intravenously. In another embodiment, the vaccine is administered orally, whereas in another embodiment, the vaccine is administered parenterally (e.g., subcutaneously, intramuscularly, and the like).

Further, in another embodiment, the compositions or vaccines are administered as a suppository, for example a rectal suppository or a urethral suppository. Further, in another embodiment, the pharmaceutical compositions are administered by subcutaneous implantation of a pellet. In a further embodiment, the pellet provides for controlled release of an agent over a period of time. In yet another embodiment, the pharmaceutical compositions are administered in the form of a capsule.

In one embodiment, the route of administration may be parenteral. In another embodiment, the route may be intra-ocular, conjunctival, topical, transdermal, intradermal, subcutaneous, intraperitoneal, intravenous, intra-arterial, vaginal, rectal, intratumoral, parcanceral, transmucosal, intramuscular, intravascular, intraventricular, intracranial, inhalation (aerosol), nasal aspiration (spray), intranasal (drops), sublingual, oral, aerosol or suppository or a combination thereof. For intranasal administration or application by inhalation, solutions or suspensions of the compounds mixed and aerosolized or nebulized in the presence of the appropriate carrier suitable. Such an aerosol may comprise any agent described herein. In one embodiment, the compositions as set forth herein may be in a form suitable for intracranial administration, which in one embodiment, is intrathecal and intracerebroventricular administration. In one embodiment, the regimen of administration will be determined by skilled clinicians, based on factors such as exact nature of the condition being treated, the severity of the condition, the age and general physical condition of the patient, body weight, and response of the individual patient, etc.

In one embodiment, parenteral application, particularly suitable are injectable, sterile solutions, preferably oily or aqueous solutions, as well as suspensions, emulsions, or implants, including suppositories and enemas. Ampoules are convenient unit dosages. Such a suppository may comprise any agent described herein.

In one embodiment, sustained or directed release compositions can be formulated, e.g., liposomes or those wherein the active compound is protected with differentially degradable coatings, e.g., by microencapsulation, multiple coatings, etc. Such compositions may be formulated for immediate or slow release. It is also possible to freeze-dry the new compounds and use the lyophilisates obtained, for example, for the preparation of products for injection.

In one embodiment, for liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

In one embodiment, compositions of this invention are pharmaceutically acceptable. In one embodiment, the term “pharmaceutically acceptable” refers to any formulation which is safe, and provides the appropriate delivery for the desired route of administration of an effective amount of at least one compound for use in the present invention. This term refers to the use of buffered formulations as well, wherein the pH is maintained at a particular desired value, ranging from pH 4.0 to pH 9.0, in accordance with the stability of the compounds and route of administration.

In one embodiment, a composition of or used in the methods of this invention may be administered alone or within a composition. In another embodiment, compositions of this invention admixture with conventional excipients, i.e., pharmaceutically acceptable organic or inorganic carrier substances suitable for parenteral, enteral (e.g., oral) or topical application which do not deleteriously react with the active compounds may be used. In one embodiment, suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. In another embodiment, the pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. In another embodiment, they can also be combined where desired with other active agents, e.g., vitamins.

In one embodiment, the compositions for use of the methods and compositions disclosed herein may be administered with a carrier/diluent. Solid carriers/diluents include, but are not limited to, a gum, a starch (e.g., corn starch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

In one embodiment, the compositions of the methods and compositions disclosed herein may comprise the composition of this invention and one or more additional compounds effective in preventing or treating cancer. In some embodiments, the additional compound may comprise a compound useful in chemotherapy, which in one embodiment, is Cisplatin. In another embodiment, Ifosfamide, Fluorouracilor5-FU, Irinotecan, Paclitaxel (Taxol), Docetaxel, Gemcitabine, Topotecan or a combination thereof, may be administered with a composition disclosed herein for use in the methods disclosed herein. In another embodiment, Amsacrine, Bleomycin, Busulfan, Capecitabine, Carboplatin, Carmustine, Chlorambucil, Cisplatin, Cladribine, Clofarabine, Crisantaspase, Cyclophosphamide, Cytarabine, Dacarbazine, Dactinomycin, Daunorubicin, Docetaxel, Doxorubicin, Epirubicin, Etoposide, Fludarabine, Fluorouracil, Gliadelimplants, Hydroxycarbamide, Idarubicin, Ifosfamide, Irinotecan, Leucovorin, Liposomaldoxorubicin, Liposomaldaunorubicin, Lomustine, Melphalan, Mercaptopurine, Mesna, Methotrexate, Mitomycin, Mitoxantrone, Oxaliplatin, Paclitaxel, Pemetrexed, Pentostatin, Procarbazine, Raltitrexed, Satraplatin, Streptozocin, Tegafur-uracil, Temozolomide, Teniposide, Thiotepa, Tioguanine, Topotecan, Treosulfan, Vinblastine, Vincristine, Vindesine, Vinorelbine, or a combination thereof, may be administered with a composition disclosed herein for use in the methods disclosed herein.

In another embodiment, fusion proteins disclosed herein are prepared by a process comprising subcloning of appropriate sequences, followed by expression of the resulting nucleotide. In another embodiment, subsequences are cloned and the appropriate subsequences cleaved using appropriate restriction enzymes. The fragments are then ligated, in another embodiment, to produce the desired DNA sequence. In another embodiment, DNA encoding the fusion protein is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The insert is then ligated into a plasmid. In another embodiment, a similar strategy is used to produce a protein wherein an HMW-MAA fragment is embedded within a heterologous peptide.

In one embodiment, the present invention also provides a recombinant Listeria comprising a nucleic acid molecule encoding a heterologous antigenic polypeptide or fragment thereof, wherein said nucleic acid molecule is operably integrated into the Listeria genome as an open reading frame with an endogenous polypeptide comprising a PEST sequence.

In one embodiment, provided herein is a recombinant Listeria expressing a heterologous antigens comprising ant antigen that is operably integrated in the genome as an open reading frame with a polypeptide or fragment thereof comprising a PEST sequence. In another embodiment, provided herein is a recombinant Listeria expressing a heterologous antigen from an episomal plasmid fused to a polypeptide or fragment thereof comprising a PEST sequence In another embodiment, the the antigen and polypeptide comprising a PEST sequence are expressed from a episomal vector present in the cytoplasm of the recombinant Listeria. In another embodiment, said polypeptide or fragment thereof is ActA, or LLO. In another embodiment, said antigen is survivin, or any other antigen provided herein. In another embodiment, said fragment is an immunogenic fragment. In yet another embodiment, said episomal expression vector lacks an antibiotic resistance marker.

In another embodiment, gene or protein expression is determined by methods that are well known in the art which in another embodiment comprise real-time PCR, northern blotting, immunoblotting, etc. In another embodiment, said first or second antigen's expression is controlled by an inducible system, while in another embodiment, said first or second antigen's expression is controlled by a constitutive promoter. In another embodiment, inducible expression systems are well known in the art.

Methods for transforming bacteria are well known in the art, and include calcium-chloride competent cell-based methods, electroporation methods, bacteriophage-mediated transduction, chemical, and physical transformation techniques (de Boer et al, 1989, Cell 56:641-649; Miller et al, 1995, FASEB J., 9:190-199; Sambrook et al. 1989, Molecular Cloning: A Laboratory Manual, Cold Spring Harbor Laboratory, New York; Ausubel et al., 1997, Current Protocols in Molecular Biology, John Wiley & Sons, New York; Gerhardt et al., eds., 1994, Methods for General and Molecular Bacteriology, American Society for Microbiology, Washington, DC; Miller, 1992, A Short Course in Bacterial Genetics, Cold Spring Harbor Laboratory Press, Cold Spring Harbor, N.Y.) In another embodiment, the Listeria vaccine strain disclosed herein is transformed by electroporation.

In another embodiment, provided herein is a method of inhibiting the onset of cancer, said method comprising the step of administering a recombinant Listeria composition that expresses the heterologous antigen provided herein and which is specifically expressed by or in said cancer.

In one embodiment, provided herein is a method of treating a tumor in a subject, said method comprising the step of administering a recombinant Listeria composition that expresses the heterologous antigen provided herein.

In another embodiment, provided herein is a method of ameliorating symptoms that are associated with a cancer in a subject, said method comprising the step of administering a recombinant Listeria composition that expresses the heterologous antigen provided herein.

In one embodiment, provided herein is a method of protecting a subject from cancer, said method comprising the step of administering a recombinant Listeria composition that expresses the heterologous antigen provided herein.

In another embodiment, provided herein is a method of delaying onset of cancer, said method comprising the step of administering a recombinant Listeria composition that expresses the heterologous antigen provided herein. In another embodiment, provided herein is a method of treating metastatic cancer, said method comprising the step of administering a recombinant Listeria composition that expresses the heterologous antigen provided herein. In another embodiment, provided herein is a method of preventing metastatic canceror micrometastatis, said method comprising the step of administering a recombinant Listeria composition that expresses the heterologous antigen provided herein. In another embodiment, the recombinant Listeria composition is administered orally, intravenously, or parenterally.

In another embodiment of the methods and compositions disclosed herein, “nucleic acids” or “nucleotide” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed.

The terms “polypeptide,” “peptide” and “recombinant peptide” refer, in another embodiment, to a peptide or polypeptide of any length. In another embodiment, a peptide or recombinant peptide disclosed herein has one of the lengths enumerated above for a survivin fragment. Each possibility represents a separate embodiment of the methods and compositions disclosed herein. In one embodiment, the term “peptide” refers to native peptides (either degradation products, synthetically synthesized peptides or recombinant peptides) and/or peptidomimetics (typically, synthetically synthesized peptides), such as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, ═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

In one embodiment, “antigenic polypeptide” is used herein to refer to a polypeptide, peptide or recombinant peptide as described hereinabove that is processed and presented on MHC class I and/or class II molecules present in a subject's cells leading to the mounting of an immune response when present in, or, in another embodiment, detected by, the host.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), *-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time. Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides disclosed herein may also include one or more modified amino acids or one or more non-amino acid monomers (e.g. fatty acids, complex carbohydrates etc).

In one embodiment, the term “oligonucleotide” is interchangeable with the term “nucleic acid”, and may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

“Stably maintained” refers, in another embodiment, to maintenance of a nucleic acid molecule or plasmid in the absence of selection (e.g. antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than 500 generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro.

In one embodiment, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

The term “nucleic acid” or “nucleic acid sequence” refers to a deoxyribonucleotide or ribonucleotide oligonucleotide in either single- or double-stranded form. The term encompasses nucleic acids, i.e., oligonucleotides, containing known analogues of natural nucleotides which have similar or improved binding properties, for the purposes desired, as the reference nucleic acid. The term also includes nucleic acids which are metabolized in a manner similar to naturally occurring nucleotides or at rates that are improved thereover for the purposes desired. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see, e.g., Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Mulligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., in WO 97/03211; WO 96/39154; Mata (1997) Toxicol. Appi. Pharmacol. 144:189-197. Other synthetic backbones encompasses by the term include methyl-phosphonate linkages or alternating methyiphosphonate and phosphodiester linkages (Strauss-Soukup (1997) Biochemistry 36:8692-8698), and benzylphosphonate linkages (Samstag (1996) Antisense Nucleic Acid Drug Dev. 6:153-156). The term nucleic acid is used interchangeably with gene, cDNA, mRNA, oligonucleotide primer, probe and amplification product.

In one embodiment of the methods and compositions disclosed herein, the term “recombination site” or “site-specific recombination site” refers to a sequence of bases in a nucleic acid molecule that is recognized by a recombinase (along with associated proteins, in some cases) that mediates exchange or excision of the nucleic acid segments flanking the recombination sites. The recombinases and associated proteins are collectively referred to as “recombination proteins” see, e.g., Landy, A., (Current Opinion in Genetics & Development) 3:699-707; 1993).

A “phage expression vector” or “phagemid” refers to any phage-based recombinant expression system for the purpose of expressing a nucleic acid sequence of the methods and compositions disclosed herein in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

In one embodiment, the term “operably linked” as used herein means that the transcriptional and translational regulatory nucleic acid, is positioned relative to any coding sequences in such a manner that transcription is initiated. Generally, this will mean that the promoter and transcriptional initiation or start sequences are positioned 5′ to the coding region.

In one embodiment, an “open reading frame” or “ORF” is a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, in one embodiment, a nucleic acid molecule operably integrated into a genome as an open reading frame with an endogenous polypeptide is a nucleic acid molecule that has integrated into a genome in the same open reading frame as an endogenous polypeptide.

In one embodiment, the present invention provides a fusion polypeptide comprising a linker sequence. In one embodiment, a “linker sequence” refers to an amino acid sequence that joins two heterologous polypeptides, or fragments or domains thereof. In general, as used herein, a linker is an amino acid sequence that covalently links the polypeptides to form a fusion polypeptide. A linker typically includes the amino acids translated from the remaining recombination signal after removal of a reporter gene from a display vector to create a fusion protein comprising an amino acid sequence encoded by an open reading frame and the display protein. As appreciated by one of skill in the art, the linker can comprise additional amino acids, such as glycine and other small neutral amino acids.

In one embodiment, “endogenous” as used herein describes an item that has developed or originated within the reference organism or arisen from causes within the reference organism. In another embodiment, endogenous refers to native.

It will be appreciated by a skilled artisan that the term “heterologous” encompasses a nucleic acid, amino acid, peptide, polypeptide, or protein derived from a different species than the reference species. Thus, for example, a Listeria strain expressing a heterologous polypeptide, in one embodiment, would express a polypeptide that is not native or endogenous to the Listeria strain, or in another embodiment, a polypeptide that is not normally expressed by the Listeria strain, or in another embodiment, a polypeptide from a source other than the Listeria strain. In another embodiment, heterologous may be used to describe something derived from a different organism within the same species. In another embodiment, the heterologous antigen is expressed by a recombinant strain of Listeria, and is processed and presented to cytotoxic T-cells upon infection of mammalian cells by the recombinant strain. In another embodiment, the heterologous antigen expressed by Listeria species need not precisely match the corresponding unmodified antigen or protein in the tumor cell or infectious agent so long as it results in a T-cell response that recognizes the unmodified antigen or protein which is naturally expressed in the mammal The term heterologous antigen may be referred to herein as “antigenic polypeptide”, “heterologous protein”, “heterologous protein antigen”, “protein antigen”, “antigen”, and the like.

It will be appreciated by the skilled artisan that the term “episomal expression vector” ecompsasses a nucleic acid vector which may be linear or circular, and which is usually double-stranded in form and is extrachromosomal in that it is present in the cytoplasm of a host bacteria or cell as opposed to being integrated into the bacteria's or cell's genome. In one embodiment, an episomal expression vector comprises a gene of interest. In another embodiment, episomal vectors persist in multiple copies in the bacterial cytoplasm, resulting in amplification of the gene of interest, and, in another embodiment, viral trans-acting factors are supplied when necessary. In another embodiment, the episomal expression vector may be referred to as a plasmid herein. In another embodiment, an “integrative plasmid” comprises sequences that target its insertion or the insertion of the gene of interest carried within into a host genome. In another embodiment, an inserted gene of interest is not interrupted or subjected to regulatory constraints which often occur from integration into cellular DNA. In another embodiment, the presence of the inserted heterologous gene does not lead to rearrangement or interruption of the cell's own important regions. In another embodiment, in stable transfection procedures, the use of episomal vectors often results in higher transfection efficiency than the use of chromosome-integrating plasmids (Belt, P.B.G.M., et al (1991) Efficient cDNA cloning by direct phenotypic correction of a mutant human cell line (HPRT2) using an Epstein-Barr virus-derived cDNA expression vector. Nucleic Acids Res. 19, 4861-4866; Mazda, 0., et al. (1997) Extremely efficient gene transfection into lympho-hematopoietic cell lines by Epstein-Barr virus-based vectors. J. Immunol. Methods 204, 143-151) In one embodiment, the episomal expression vectors of the methods and compositions disclosed herein may be delivered to cells in vivo, ex vivo, or in vitro by any of a variety of the methods employed to deliver DNA molecules to cells. The vectors may also be delivered alone or in the form of a pharmaceutical composition that enhances delivery to cells of a subject.

In one embodiment, the term “fused” refers to operable linkage by covalent bonding. In one embodiment, the term includes recombinant fusion (of nucleic acid sequences or open reading frames thereof). In another embodiment, the term includes chemical conjugation.

“Transforming,” in one embodiment, refers to engineering a bacterial cell to take up a plasmid or other heterologous DNA molecule. In another embodiment, “transforming” refers to engineering a bacterial cell to express a gene of a plasmid or other heterologous DNA molecule.

In another embodiment, conjugation is used to introduce genetic material and/or plasmids into bacteria. Methods for conjugation are well known in the art, and are described, for example, in Nikodinovic J et al (A second generation snp-derived Escherichia coli-Streptomyces shuttle expression vector that is generally transferable by conjugation. Plasmid. 2006 Nov;56(3):223-7) and Auchtung JM et al (Regulation of a Bacillus subtilis mobile genetic element by intercellular signaling and the global DNA damage response. Proc Natl Acad Sci U S A. 2005 Aug 30;102(35):12554-9).

“Metabolic enzyme” refers, in another embodiment, to an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient.

In one embodiment, the term “attenuation,” as used herein, is meant a diminution in the ability of the bacterium to cause disease in an animal. In other words, the pathogenic characteristics of the attenuated Listeria strain have been lessened compared with wild-type Listeria, although the attenuated Listeria is capable of growth and maintenance in culture. Using as an example the intravenous inoculation of Balb/c mice with an attenuated Listeria, the lethal dose at which 50% of inoculated animals survive (LD₅₀) is preferably increased above the LD₅₀ of wild-type Listeria by at least about 10-fold, more preferably by at least about 100-fold, more preferably at least about 1,000 fold, even more preferably at least about 10,000 fold, and most preferably at least about 100,000-fold. An attenuated strain of Listeria is thus one which does not kill an animal to which it is administered, or is one which kills the animal only when the number of bacteria administered is vastly greater than the number of wild type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. The attenuated strains of the present invention are therefore environmentally safe in that they are incapable of uncontrolled replication.

In one embodiment, the Listeria disclosed herein expresses a heterologous polypeptide, as described herein, in another embodiment, the Listeria disclosed herein secretes a heterologous polypeptide, as described herein, and in another embodiment, the Listeria disclosed herein expresses and secretes a heterologous polypeptide, as described herein. In another embodiment, the Listeria disclosed herein comprises a heterologous polypeptide, and in another embodiment, comprises a nucleic acid that encodes a heterologous polypeptide.

In one embodiment, Listeria strains disclosed herein may be used in the preparation of vaccines. In one embodiment, Listeria strains disclosed herein may be used in the preparation of peptide vaccines. Methods for preparing peptide vaccines are well known in the art and are described, for example, in EP1408048, United States Patent Application Number 20070154953, and OGASAWARA et al (Proc. Nati. Acad. Sci. USA Vol. 89, pp. 8995-8999, October 1992). In one embodiment, peptide evolution techniques are used to create an antigen with higher immunogenicity. Techniques for peptide evolution are well known in the art and are described, for example in U.S. Pat. No. 6,773,900.

In one embodiment, the vaccines of the methods and compositions disclosed herein may be administered to a host vertebrate animal, preferably a mammal, and more preferably a human, either alone or in combination with a pharmaceutically acceptable carrier. In another embodiment, the vaccine is administered in an amount effective to induce an immune response to the Listeria strain itself or to a heterologous antigen which the Listeria species has been modified to express. In another embodiment, the amount of vaccine to be administered may be routinely determined by one of skill in the art when in possession of the present disclosure. In another embodiment, a pharmaceutically acceptable carrier may include, but is not limited to, sterile distilled water, saline, phosphate buffered solutions or bicarbonate buffered solutions. In another embodiment, the pharmaceutically acceptable carrier selected and the amount of carrier to be used will depend upon several factors including the mode of administration, the strain of Listeria and the age and disease state of the vaccinee. In another embodiment, administration of the vaccine may be by an oral route, or it may be parenteral, intranasal, intramuscular, intravascular, intrarectal, intraperitoneal, or any one of a variety of well-known routes of administration. In another embodiment, the route of administration may be selected in accordance with the type of infectious agent or tumor to be treated.

In one embodiment, the present invention provides a recombinant Listeria strain comprising a nucleic acid molecule encoding a heterologous antigenic polypeptide or fragment thereof, wherein said nucleic acid molecule is operably integrated into the Listeria genome in an open reading frame with an endogenous PEST-containing gene. In another embodiment, the nucleic acid molecule is expressed from an episomal plasmid.

In another embodiment, the present invention provides a method of inducing an immune response to an antigen in a subject comprising administering a recombinant Listeria strain comprising a nucleic acid molecule encoding a heterologous antigenic polypeptide or fragment thereof, wherein said nucleic acid molecule is operably integrated into the Listeria genome in an open reading frame with an endogenous PEST-containing gene. In another embodiment, the nucleic acid molecule is expressed from an episomal plasmid.

In another embodiment, the present invention provides a method of treating, suppressing, or inhibiting a cancer in a subject comprising administering a recombinant Listeria strain comprising a nucleic acid molecule encoding a heterologous antigenic polypeptide or fragment thereof, wherein said nucleic acid molecule is operably integrated into the Listeria genome in an open reading frame with an endogenous PEST-containing gene. In another embodiment, the nucleic acid molecule is expressed from an episomal plasmid.

In another embodiment, the present invention provides a method of treating, suppressing, or inhibiting at least one tumor in a subject comprising administering a recombinant Listeria strain comprising a nucleic acid molecule encoding a heterologous antigenic polypeptide or fragment thereof, wherein said nucleic acid molecule is operably integrated into the Listeria genome in an open reading frame with an endogenous PEST-containing gene. In another embodiment, the nucleic acid molecule is expressed from an episomal plasmid.

In another embodiment, the present invention provides a method of producing a recombinant Listeria strain expressing an antigen, the method comprising genetically fusing a nucleic acid encoding an antigen into the Listeria genome in an open reading frame with an endogenous PEST-containing gene; and expressing said antigen under conditions conducive to antigenic expression in said recombinant Listeria strain. In another embodiment, the nucleic acid molecule is expressed from an episomal plasmid.

In another embodiment, the present invention provides any of the methods described hereinabove using a recombinant Listeria strain comprising a nucleic acid molecule encoding a heterologous antigenic polypeptide or fragment thereof, wherein said nucleic acid molecule is operably integrated into the Listeria genome in an open reading frame with an endogenous PEST-containing gene. In another embodiment, the nucleic acid molecule is expressed from an episomal plasmid in an open reading frame with an endogenous PEST-containing gene.

In another embodiment, the present invention provides a kit for conveniently practicing the methods disclosed herein comprising one or more Listeria strains disclosed herein, an applicator, and instructional material that describes how to use the kit components in practicing the methods disclosed herein.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, pets mice and humans. The subject may also include livestock. In one embodiment, the term “subject” does not exclude an individual that is healthy in all respects and does not have or show signs of disease or disorder.

The following examples are presented in order to more fully illustrate the preferred embodiments of the invention. They should in no way be construed, however, as limiting the broad scope of the invention.

### EXAMPLES

A recombinant Lm that secretes PSA fused to tLLO (Lm-LLO-PSA) was developed. This strain elicits a potent PSA-specific immune response associated with regression of tumors in a mouse model for prostate cancer, wherein the expression of tLLO-PSA is derived from a plasmid based on pGG55 (Table 1), which confers antibiotic resistance to the vector. We recently developed a new strain for the PSA vaccine based on the pADV142 plasmid, which has no antibiotic resistance markers, and referred as LmddA -142 (Table 1). This new strain is 10 times more attenuated than Lm-LLO-PSA. In addition, LmddA-142 was slightly more immunogenic and significantly more efficacious in regressing PSA expressing tumors than the Lm-LLO-PSA.

TABLE 1
Plasmids and strains

| Plasmids | Features |
|---|---|
| pGG55 | pAM401/pGB354 shuttle plasmid with gram(−) and |
| | gram(+) cm resistance, LLO-E7 expression cassette and a |
| | copy of Lm prfA gene |
| pTV3 | Derived from pGG55 by deleting cm genes and inserting |
| | the Lm dal gene |
| pADV119 | Derived from pTV3 by deleting the prfA gene |
| pADV134 | Derived from pADV119 by replacing the Lm dal gene |
| | by the Bacillus dal gene |
| pADV142 | Derived from pADV134 by replacing HPV16 e7 with |
| | klk3 |
| pADV168 | Derived from pADV134 by replacing HPV16 e7 with |
| | hmw-maa₂₁₆₀₋₂₂₅₈ |
| Strains | Genotype |
| 10403S | Wild-type Listeria monocytogenes:: str |
| XFL-7 | 10403S prfA⁽⁻⁾ |
| Lmdd | 10403S dal⁽⁻⁾ dat⁽⁻⁾ |
| LmddA | 10403S dal⁽⁻⁾ dat⁽⁻⁾ actA⁽⁻⁾ |
| LmddA-134 | 10403S dal⁽⁻⁾ dat⁽⁻⁾ actA⁽⁻⁾ pADV134 |
| LmddA-142 | 10403S dal⁽⁻⁾ dat⁽⁻⁾ actA⁽⁻⁾ pADV142 |
| Lmdd-143 | 10403S dal⁽⁻⁾ dat⁽⁻⁾ with klk3 fused to the hly gene in the |
| | chromosome |
| LmddA-143 | 10403S dal⁽⁻⁾ dat⁽⁻⁾ actA⁽⁻⁾ with klk3 fused to the hly gene |
| | in the chromosome |
| LmddA-168 | 10403S dal⁽⁻⁾ dat⁽⁻⁾ actA⁽⁻⁾ pADV168 |
| Lmdd-143/134 | Lmdd-143 pADV134 |
| LmddA- | LmddA-143 pADV134 |
| 143/134 | |
| Lmdd-143/168 | Lmdd-143 pADV168 |
| LmddA- | LmddA-143 pADV168 |
| 143/168 | |

The sequence of the plasmid pAdv142 (6523 bp) was as follows:

(SEQ ID NO: 28)
cggagtgtatactggcttactatgttggcactgatgagggtgtcagtgaa
gtgcttcatgtggcaggagaaaaaaggctgcaccggtgcgtcagcagaat
atgtgatacaggatatattccgcacctcgctcactgactcgctacgctcg
gtcgttcgactgcggcgagcggaaatggcttacgaacggggcggagattt
cctggaagatgccaggaagatacttaacagggaagtgagagggccgcggc
aaagccgataccataggctccgcccccctgacaagcatcacgaaatctga
cgctcaaatcagtggtggcgaaacccgacaggactataaagataccaggc
gtttccccctggcggctccctcgtgcgctctcctgttcctgcctttcggt
ttaccggtgtcattccgctgttatggccgcgtagtctcattccacgcctg
acactcagaccgggtaggcagttcgctccaagctggactgtatgcacgaa
ccccccgttcagtccgaccgctgcgccttatccggtaactatcgtcttga
gtccaacccggaaagacatgcaaaagcaccactggcagcagccactggta
attgatttagaggagttagtcttgaagtcatgcgccggttaaggctaaac
tgaaaggacaagttttggtgactgcgctcctccaagccagttacctcggt
tcaaagagttggtagctcagagaaccttcgaaaaaccgccctgcaaggcg
gttttttcgttttcagagcaagagattacgcgcagaccaaaacgatctca
agaagatcatcttattaatcagataaaatatactagccctcattgattag
tatattcctatcttaaagttactatatgtggaggcattaacatagttaat
gacgtcaaaaggatagcaagactagaataaagctataaagcaagcatata
atattgcgatcatattagaagcgaatttcgccaatattataattatcaaa
agagaggggtggcaaacggtataggcattattaggttaaaaaatgtagaa
ggagagtgaaacccatgaaaaaaataatgctagtattattacacttatat
tagttagtctaccaattgcgcaacaaactgaagcaaaggatgcatctgca
ttcaataaagaaaattcaatttcatccatggcaccaccagcatctccgcc
tgcaagtcctaagacgccaatcgaaaagaaacacgcggatgaaatcgata
agtatatacaaggattggattacaataaaaacaatgtattagtataccac
ggagatgcagtgacaaatgtgccgccaagaaaaggttacaaagatggaaa
tgaatatattgagtggagaaaaagaagaaatccatcaatcaaaataatgc
agacattcaagagtgaatgcaatttcgagcctaacctatccaggtgctct
cgtaaaagcgaattcggaattagtagaaaatcaaccagatgactccctgt
aaaacgtgattcattaacactcagcattgatttgccaggtatgactaatc
aagacaataaaatagagtaaaaaatgccactaaatcaaacgttaacaacg
cagtaaatacattagtggaaagatggaatgaaaaatatgctcaagcttat
ccaaatgtaagtgcaaaaattgattatgatgacgaaatggcttacagtga
atcacaattaattgcgaaataggtacagcatttaaagctgtaaataatag
cttgaatgtaaacttcggcgcaatcagtgaagggaaaatgcaagaagaag
tcattagattaaacaaatttactataacgtgaatgttaatgaacctacaa
gaccaccagattatcggcaaagctgttactaaagagcagagcaagcgctt
ggagtgaatgcagaaaatcctcctgcatatatctcaagtgtggcgtatgg
ccgtcaagtttatttgaaattatcaactaattcccatagtactaaagtaa
aagctgcttttgatgctgccgtaagcggaaaatctgtctcaggtgatgta
gaactaacaaatatcatcaaaaattcaccacaaagccgtaatttacggag
gaccgcaaaagatgaagttcaaatcatcgacggcaacctcggagacttac
gcgatattagaaaaaaggcgctactataatcgagaaacaccaggagaccc
attgcttatacaacaaacttcctaaaagacaatgaattagctgttattaa
aaacaactcagaatatattgaaacaacttcaaaagcttatacagatggaa
aaattaacatcgatcactctggaggatacgttgctcaattcaacatactt
gggatgaagtaaattatgatctcgagattgtgggaggctgggagtgcgag
aagcattcccaaccctggcaggtgcttgtggcctctcgtggcagggcagt
ctgcggcggtgactggtgcacccccagtgggtcctcacagctgcccactg
catcaggaacaaaagcgtgatcttgctgggtcggcacagcctgatcatcc
tgaagacacaggccaggtatttca2gtcagccacagcacccacacccgct
ctacgatatgagcctcctgaagaatcgattcctcaggccaggtgatgact
ccagccacgacctcatgctgctccgcctgtcagagcctgccgagctcacg
gatgctgtgaaggtcatggacctgcccacccaggagccagcactggggac
cacctgctacgcctcaggctggggcagcattgaaccagaggagacttgac
cccaaagaaacttcagtgtgtggacctccatgttatttccaatgacgtgt
gtgcgcaagttcaccctcagaaggtgaccaagttcatgctgtgtgctgga
cgctggacagggggcaaaagcacctgctcgggtgattctgggggcccact
tgtctgttatggtgtgcttcaaggtatcacgtcatggggcagtgaaccat
gtgccctgcccgaaaggccaccctgtacaccaaggtggtgcattaccgga
agtggatcaaggacaccatcgtggccaaccccTAAcccgggccactaact
caacgctagtagtggatttaatcccaaatgagccaacagaaccagaacca
gaaacagaacaagtaacattggagttagaaatggaagaagaaaaaagcaa
tgatttcgtgtgaataatgcacgaaatcattgcttattatttaaaaagcg
atatactagatataacgaaacaacgaactgaataaagaatacaaaaaaag
agccacgaccagttaaagcctgagaaactttaactgcgagccttaattga
ttaccaccaatcaattaaagaagtcgagacccaaaataggtaaagtattt
aattactttattaatcagatacttaaatatctgtaaacccattatatcgg
gatttgaggggatttcaagtattaagaagataccaggcaatcaattaaga
aaaacttagttgattgccattagagtgattcaactagatcgtagcactaa
ctaattaattacgtaagaaaggagaacagctgaatgaatatcccattgag
tagaaactgtgcttcatgacggcagttaaagtacaaatttaaaaatagta
aaattcgctcaatcactaccaagccaggtaaaagtaaaggggctattttt
gcgtatcgctcaaaaaaaagcatgattggcggacgtggcgttgactgact
tccgaagaagcgattcacgaaaatcaagatacatttacgcattggacacc
aaacgatatcgttatggtacgtatgcagacgaaaaccgttcatacactaa
aggacattctgaaaacaatttaagacaaatcaataccactttattgatta
gatattcacacggaaaaagaaactatttcagcaagcgatatataacaaca
gctattgatttaggattatgcctacgttaattatcaaatctgataaaggt
tatcaagcatattagattagaaacgccagtctatgtgacttcaaaatcag
aatttaaatctgtcaaagcagccaaaataatctcgcaaaatatccgagaa
tattaggaaagtattgccagttgatctaacgtgcaatcattagggattgc
tcgtataccaagaacggacaatgtagaattttttgatcccaattaccgtt
attctttcaaagaatggcaagattggtctttcaaacaaacagataataag
ggctttactcgttcaagtctaacggattaagcggtacagaaggcaaaaaa
caagtagatgaaccctggataatctcttattgcacgaaacgaaattacag
gagaaaagggatagtagggcgcaatagcgttatgataccctctattagcc
tactttagttcaggctattcaatcgaaacgtgcgaatataatatgatgag
ataataatcgattagatcaacccttagaagaaaaagaagtaatcaaaatt
gttagaagtgcctattcagaaaactatcaaggggctaatagggaatacat
taccattattgcaaagcagggtatcaagtgatttaaccagtaaagattta
tagtccgtcaagggtggtttaaattcaagaaaaaaagaagcgaacgtcaa
cgtgttcatttgtcagaatggaaagaagatttaatggcttatattagcga
aaaaagcgatgtatacaagccttatttagcgacgaccaaaaaagagatta
gagaagtgctaggcattcctgaacggacattagataaattgctgaaggta
ctgaaggcgaatcaggaaattactttaagattaaaccaggaagaaatggt
ggcattcaacttgctagtgttaaatcattgagctatcgatcattaaatta
aaaaaagaagaacgagaaagctatataaaggcgctgacagcttcgataat
ttagaacgtacatttattcaagaaactctaaacaaattggcagaacgccc
caaaacggacccacaactcgatttgatagctacgatacaggctgaaaata
aaacccgcactatgccattacatttatatctatgatacgtgatgatacta
gctggctagcttaattgcttatatttacctgcaataaaggatacttactt
ccattatactcccattttccaaaaacatacggggaacacgggaacttatt
gtacaggccacctcatagttaatggatcgagccacctgcaatctcatcca
tggaaatatattcatccccctgccggcctattaatgtgacttagtgcccg
gcggatattcctgatccagctccaccataaattggtccatgcaaattcgg
ccggcaattacaggcgattcccttcacaaggatgtcggtccattcaatta
cggagccagccgtccgcatagcctacaggcaccgtcccgatccatgtgta
ttaccgctgtgtactcggctccgtagctgacgctctcgccattctgatca
gatgacatgtgacagtgtcgaatgcagggtaaatgccggacgcagctgaa
acggtatctcgtccgacatgtcagcagacgggcgaaggccatacatgccg
atgccgaatctgactgcattaaaaaagccattacagccggagtccagcgg
cgctgacgcgcagtggaccattagattcataacggcagcggagcaatcag
ctattaaagcgctcaaactgcattaagaaatagcctctactattcatccg
ctgtcgcaaaatgggtaaatacccattgcactttaaacgagggagcggtc
aagaattgccatcacgactgaacttcacctctgatttacaccaagtctga
catccccgtatcgaccacagatgaaaatgaagagaaccttttttcgtgtg
gcgggctgcctcctgaagccattcaacagaataacctgttaaggtcacgt
catactcagcagcgattgccacatactccgggggaaccgcgccaagcacc
aatataggcgcatcaatccattagcgcagtgaaatcgcttcatccaaaat
ggccacggccaagcatgaagcacctgcgtcaagagcagcctttgctgttt
ctgcatcaccatgcccgtaggcgtttgctttcacaactgccatcaagtgg
acatgacaccgatatgattacatattgctgacattaccatatcgcggaca
agtcaataccgcccacgtatctctgtaaaaaggattgtgctcatggaaaa
ctcctctcattacagaaaatcccagtacgtaattaagtatttgagaatta
atatatattgattaatactaagatacccagattcacctaaaaaacaaatg
atgagataatagctccaaaggctaaagaggactataccaactatagttaa
ttaa.

This plasmid was sequenced at Genewiz facility from the E. coli strain.

### Example 1

### Construction of Attenuated Listeria Strain-LmddΔactA and Insertion of the Human klk3 Gene in Frame to the hly Gene in the Lmdd and Lmdda Strains

The strain Lm dal dat (Lmdd) was attenuated by the irreversible deletion of the virulence factor, ActA. An in-frame deletion of actA in the Lmdaldat (Lmdd) background was constructed to avoid any polar effects on the expression of downstream genes. The Lm dal dat ΔactA contains the first 19 amino acids at the N-terminal and 28 amino acid residues of the C-terminal with a deletion of 591 amino acids of ActA.

The actA deletion mutant was produced by amplifying the chromosomal region corresponding to the upstream (657 bp-oligo's Adv 271/272) and downstream (625 bp-oligo's Adv 273/274) portions of actA and joining by PCR. The sequence of the primers used for this amplification is given in the Table 2. The upstream and downstream DNA regions of actA were cloned in the pNEB193 at the EcoRI/PstI restriction site and from this plasmid, the EcoRI/PstI was further cloned in the temperature sensitive plasmid pKSV7, resulting in ΔactA/pKSV7 (pAdv120).

TABLE 2
Sequence of primers that was used for the amplification of DNA
sequences upstream and downstream of actA

| | | SEQ ID |
|---|---|---|
| Primer | Sequence | NO: |
| Adv271-actAF1 | cg GAATTCGGATCCgcgccaaatcattggttgattg | 29 |
| Adv272- | gcgaGTCGACgtcggggttaatcgtaatgcaattggc | 30 |
| actAR1 | | |
| Adv273-actAF2 | gcgaGTCGACccatacgacgttaattcttgcaatg | 31 |
| Adv274- | gataCTGCAGGGATCCttcccttctcggtaatcagtcac | 32 |
| actAR2 | | |

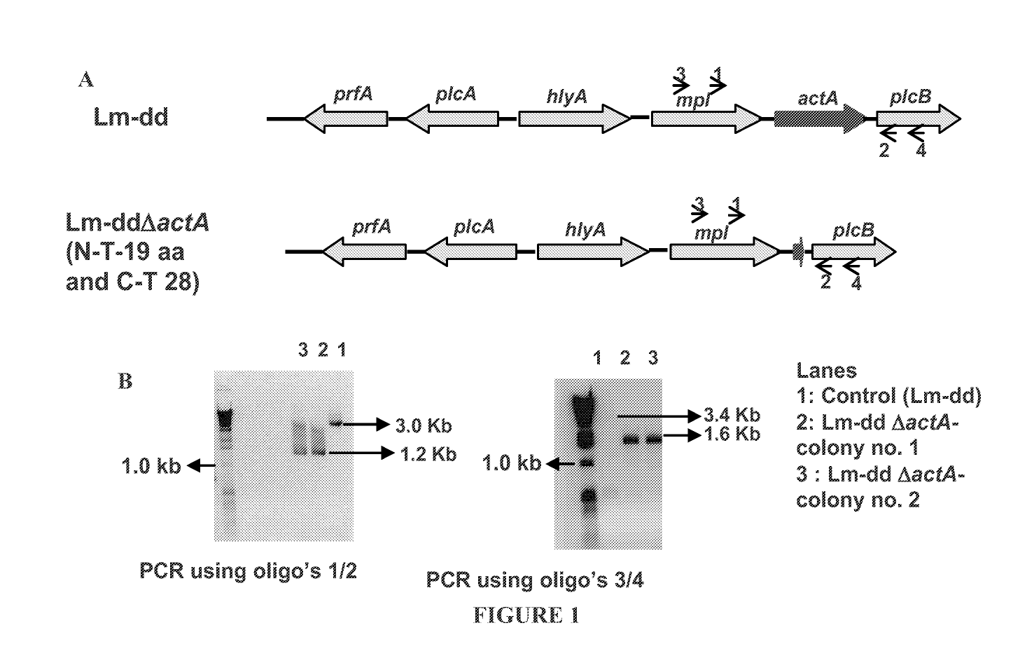

The deletion of the gene from its chromosomal location was verified using primers that bind externally to the actA deletion region, which are shown in FIG. 1 as primer 3 (Adv 305-tgggatggccaagaaattc, SEQ ID NO: 33) and primer 4 (Adv304-ctaccatgtcttccgttgcttg; SEQ ID NO: 34. The PCR analysis was performed on the chromosomal DNA isolated from Lmdd and LmddΔactA. The sizes of the DNA fragments after amplification with two different sets of primer pairs 1/2 and 3/4 in Lmdd chromosomal DNA was expected to be 3.0 Kb and 3.4 Kb. On the other hand, the expected sizes of PCR using the primer pairs 1/2 and 3/4 for the LmddΔactA was 1.2 Kb and 1.6 Kb. Thus, PCR analysis in FIG. 1 confirms that the 1.8 kb region of actA was deleted in the LmddΔactA strain. DNA sequencing was also performed on PCR products to confirm the deletion of actA containing region in the strain, LmddΔactA.

### Example 2

### Construction of the Antibiotic-Independent Episomal Expression System for Antigen Delivery by Lm Vectors

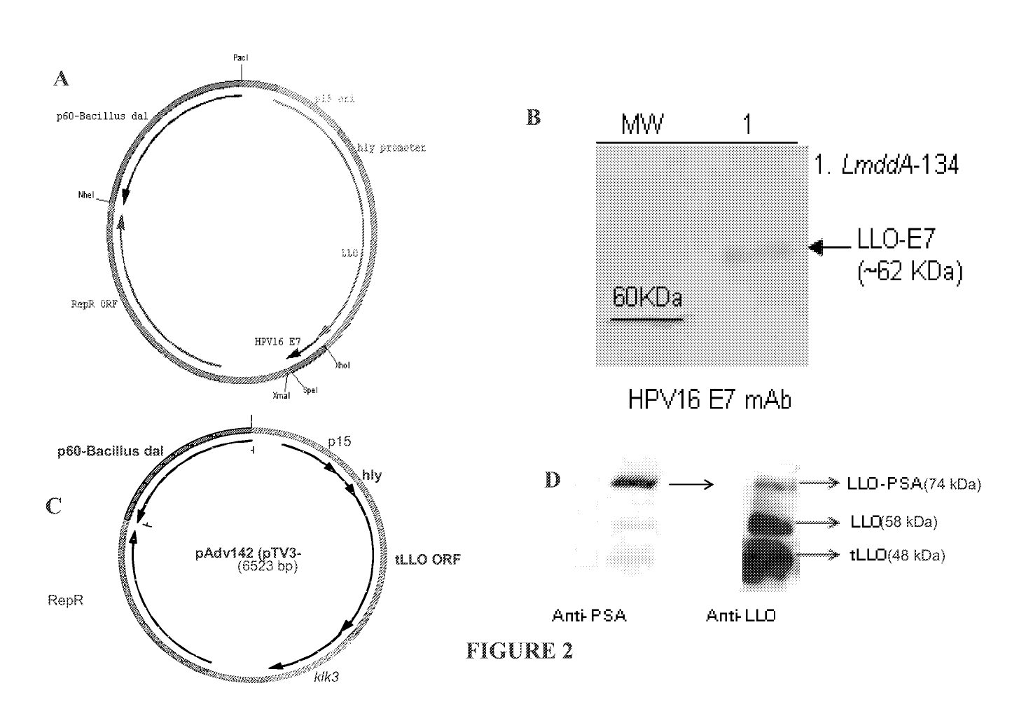

The antibiotic-independent episomal expression system for antigen delivery by Lm vectors (pAdv142) is the next generation of the antibiotic-free plasmid pTV3 (Verch et al., Infect Immun, 2004. 72(11):6418-25, incorporated herein by reference). The gene for virulence gene transcription activator, prfA was deleted from pTV3 since Listeria strain Lmdd contains a copy of prfA gene in the chromosome. Additionally, the cassette for p60-Listeria dal at the NheI/PacI restriction site was replaced by p60-Bacillus subtilis dal resulting in plasmid pAdv134 (FIG. 2A). The similarity of the Listeria and Bacillus dal genes is ˜30%, virtually eliminating the chance of recombination between the plasmid and the remaining fragment of the dal gene in the Lmdd chromosome. The plasmid pAdv134 contained the antigen expression cassette tLLO-E7. The LmddA strain was transformed with the pADV134 plasmid and expression of the LLO-E7 protein from selected clones confirmed by Western blot (FIG. 2B). The Lmdd system derived from the 10403S wild-type strain lacks antibiotic resistance markers, except for the Lmdd streptomycin resistance.

Further, pAdv134 was restricted with XhoI/XmaI to clone human PSA, klk3 resulting in the plasmid, pAdv142. The new plasmid, pAdv142 (FIG. 2C, Table 1) contains Bacillus dal (B-Dal) under the control of Listeria p60 promoter. The shuttle plasmid, pAdv142 complemented the growth of both E. coli ala drx MB2159 as well as Listeria monocytogenes strain Lmdd in the absence of exogenous D-alanine. The antigen expression cassette in the plasmid pAdv142 consists of hly promoter and LLO-PSA fusion protein (FIG. 2C).

The plasmid pAdv142 was transformed to the Listeria background strains, LmddactA strain resulting in Lm-ddA-LLO-PSA. The expression and secretion of LLO-PSA fusion protein by the strain, Lm-ddA-LLO-PSA was confirmed by Western Blot using anti-LLO and anti-PSA antibody (FIG. 2D). There was stable expression and secretion of LLO-PSA fusion protein by the strain, Lm-ddA-LLO-PSA after two in vivo passages.

### Example 3

### In vitro and in vivo Stability of the Strain LmddA-LLO-PSA

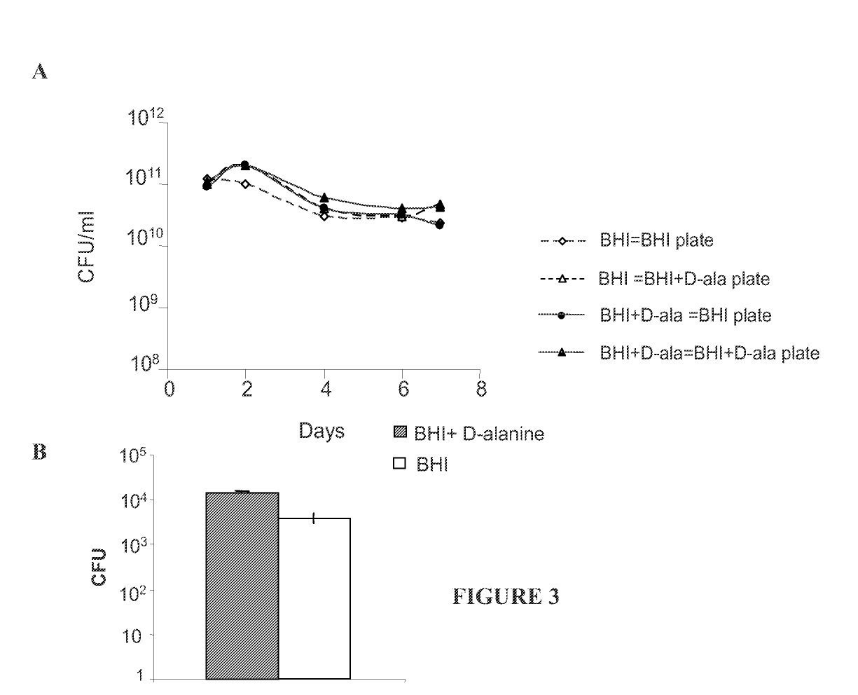

The in vitro stability of the plasmid was examined by culturing the LmddA-LLO-PSA Listeria strain in the presence or absence of selective pressure for eight days. The selective pressure for the strain LmddA-LLO-PSA is D-alanine. Therefore, the strain LmddA-LLO-PSA was passaged in Brain-Heart Infusion (BHI) and BHI+ 100 μg/ml D-alanine. CFUs were determined for each day after plating on selective (BHI) and non-selective (BHI+D-alanine) medium. It was expected that a loss of plasmid will result in higher CFU after plating on non-selective medium (BHI+D-alanine). As depicted in FIG. 3A, there was no difference between the number of CFU in selective and non-selective medium. This suggests that the plasmid pAdv142 was stable for at least 50 generations, when the experiment was terminated.

Plasmid maintenance in vivo was determined by intravenous injection of 5×10⁷ CFU LmddA-LLO-PSA, in C57BL/6 mice. Viable bacteria were isolated from spleens homogenized in PBS at 24 h and 48 h. CFUs for each sample were determined at each time point on BHI plates and BHI+100 μg/ml D-alanine. After plating the splenocytes on selective and non-selective medium, the colonies were recovered after 24 h. Since this strain is highly attenuated, the bacterial load is cleared in vivo in 24 h. No significant differences of CFUs were detected on selective and non-selective plates, indicating the stable presence of the recombinant plasmid in all isolated bacteria (FIG. 3B).

### Example 4

### In vivo Passaging, Virulence and Clearance of the Strain LmddA-142 (LmddA-LLO-PSA)

LmddA-142 is a recombinant Listeria strain that secretes the episomally expressed tLLO-PSA fusion protein. To determine a safe dose, mice were immunized with LmddA-LLO-PSA at various doses and toxic effects were determined. LmddA-LLO-PSA caused minimum toxic effects (data not shown). The results suggested that a dose of 10⁸ CFU of LmddA-LLO-PSA was well tolerated by mice. Virulence studies indicate that the strain LmddA-LLO-PSA was highly attenuated.

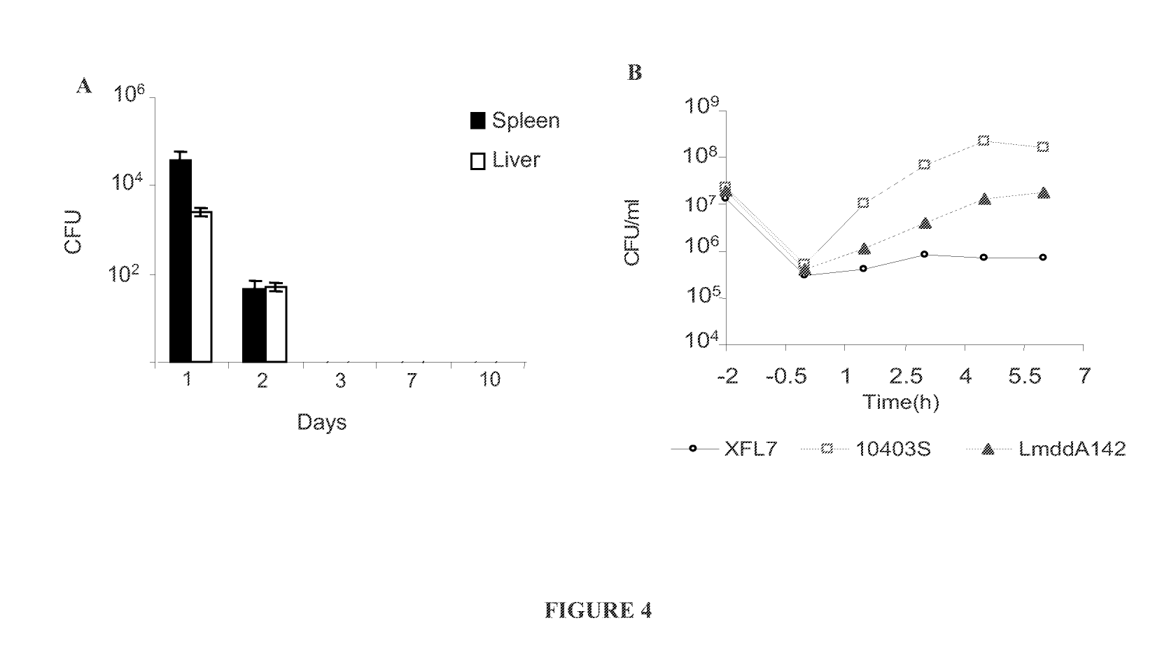

The in vivo clearance of LmddA-LLO-PSA after administration of the safe dose, 10⁸ CFU intraperitoneally in C57BL/6 mice, was determined. There were no detectable colonies in the liver and spleen of mice immunized with LmddA-LLO-PSA after day 2. Since this strain is highly attenuated, it was completely cleared in vivo at 48 h (FIG. 4A).

To determine if the attenuation of LmddA-LLO-PSA attenuated the ability of the strain LmddA-LLO-PSA to infect macrophages and grow intracellularly, we performed a cell infection assay. Mouse macrophage-like cell line such as J774A.1 were infected in vitro with Listeria constructs and intracellular growth was quantified. The positive control strain, wild type Listeria strain 10403S grows intracellularly, and the negative control XFL7, a prfA mutant, cannot escape the phagolysosome and thus does not grow in J774 cells. The intracytoplasmic growth of LmddA-LLO-PSA was slower than 10403S due to the loss of the ability of this strain to spread from cell to cell (FIG. 4B). The results indicate that LmddA-LLO-PSA has the ability to infect macrophages and grow intracytoplasmically.

### Example 5

### Immunogenicity of the Strain-LmddA-LLO-PSA in C57BL/6 Mice

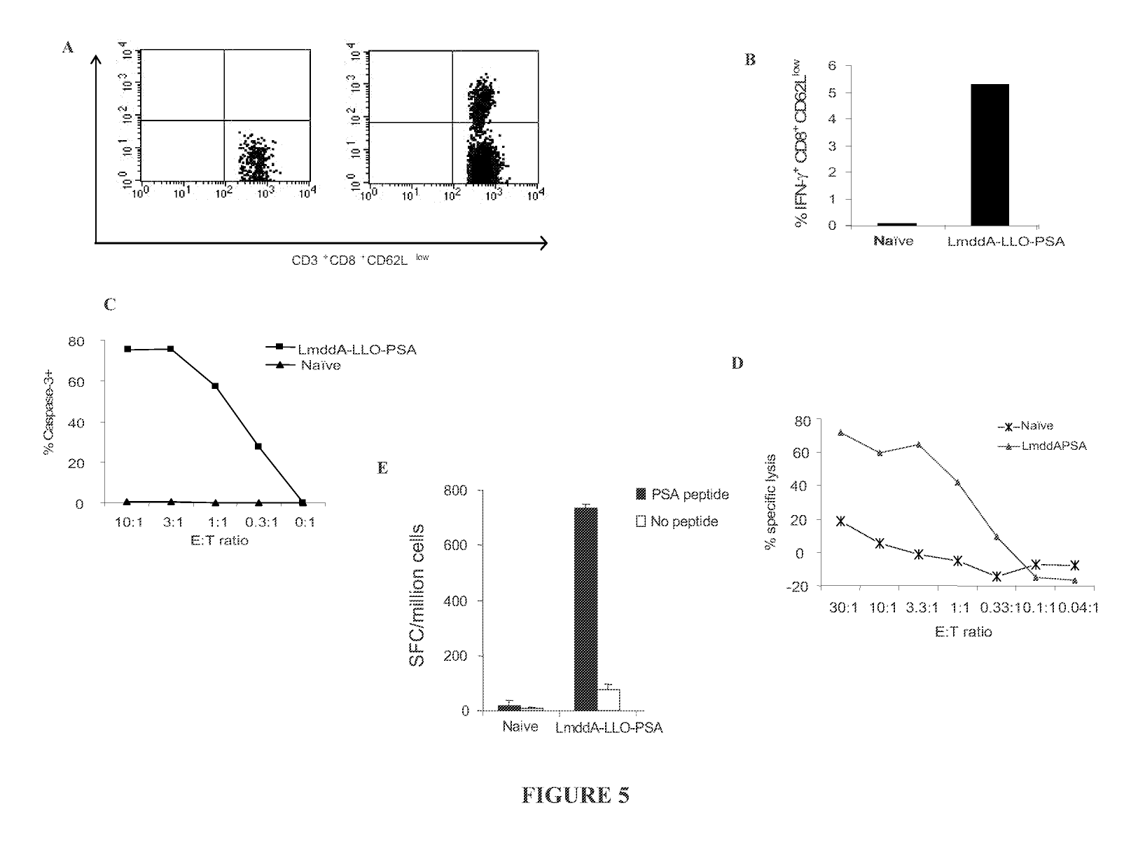

The PSA-specific immune responses elicited by the construct LmddA-LLO-PSA in C57BL/6 mice were determined using PSA tetramer staining. Mice were immunized twice with LmddA-LLO-PSA at one week intervals and the splenocytes were stained for PSA tetramer on day 6 after the boost. Staining of splenocytes with the PSA-specific tetramer showed that LmddA-LLO-PSA elicited 23% of PSA tetramer⁺CD8⁺CD62L^{low} cells (FIG. 5A).

The functional ability of the PSA-specific T cells to secrete IFN-γ after stimulation with PSA peptide for 5 h was examined using intracellular cytokine staining. There was a 200-fold increase in the percentage of CD8⁺CD62L^{low}IFN-γ secreting cells stimulated with PSA peptide in the LmddA-LLO-PSA group compared to the naive mice (FIG. 5B), indicating that the LmddA-LLO-PSA strain is very immunogenic and primes high levels of functionally active PSA CD8⁺ T cell responses against PSA in the spleen.

To determine the functional activity of cytotoxic T cells generated against PSA after immunizing mice with LmddA-LLO-PSA, we tested the ability of PSA-specific CTLs to lyse cells EL4 cells pulsed with H-2D^{b} peptide in an in vitro assay. A FACS-based caspase assay (FIG. 5C) and Europium release (FIG. 5D) were used to measure cell lysis. Splenocytes of mice immunized with LmddA-LLO-PSA contained CTLs with high cytolytic activity for the cells that display PSA peptide as a target antigen.

Elispot was performed to determine the functional ability of effector T cells to secrete IFN-γ after 24 h stimulation with antigen. Using ELISpot, we observed there was a 20-fold increase in the number of spots for IFN-γ in splenocytes from mice immunized with LmddA-LLO-PSA stimulated with specific peptide when compared to the splenocytes of the naïve mice (FIG. 5E).

### Example 6

### Immunization with the LmddA -142 Strains Induces Regression of a Tumor Expressing PSA and Infiltration of the Tumor by PSA-Specific CTLs

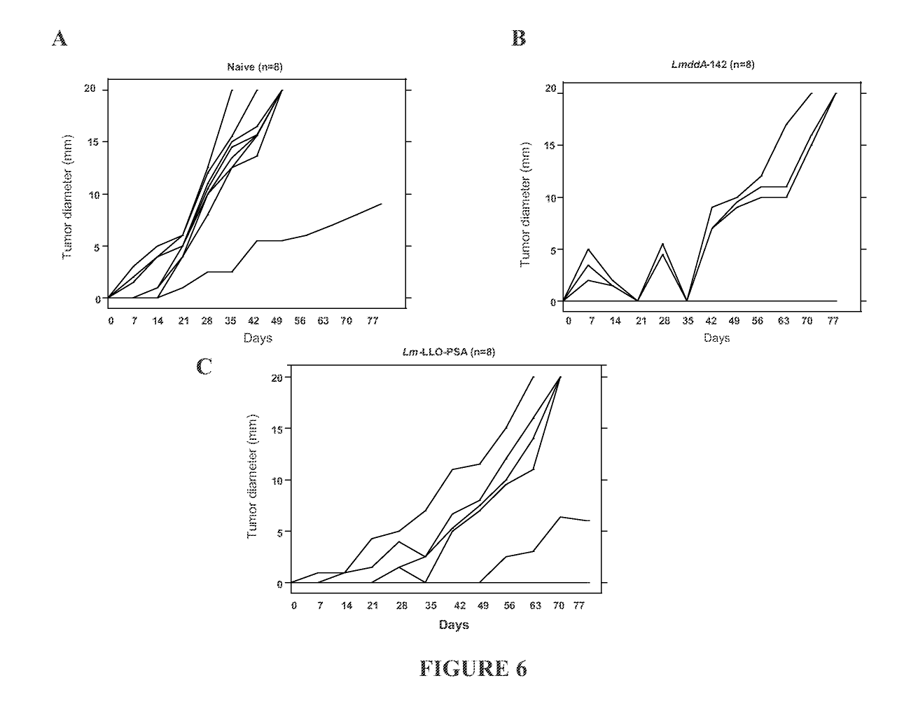

The therapeutic efficacy of the construct LmddA-142 (LmddA-LLO-PSA) was determined using a prostrate adenocarcinoma cell line engineered to express PSA (Tramp-C1-PSA (TPSA); Shahabi et al., 2008). Mice were subcutaneously implanted with 2×10⁶ TPSA cells. When tumors reached the palpable size of 4-6 mm, on day 6 after tumor inoculation, mice were immunized three times at one week intervals with 10⁸ CFU LmddA-142, 10⁷ CFU Lm-LLO-PSA (positive control) or left untreated. The naive mice developed tumors gradually (FIG. 6A). The mice immunized with LmddA-142 were all tumor-free until day 35 and gradually 3 out of 8 mice developed tumors, which grew at a much slower rate as compared to the naive mice (FIG. 6B). Five out of eight mice remained tumor free through day 70. As expected, Lm-LLO-PSA-vaccinated mice had fewer tumors than naïve controls and tumors developed more slowly than in controls (FIG. 6C). Thus, the construct LmddA-LLO-PSA could regress 60% of the tumors established by TPSA cell line and slow the growth of tumors in other mice. Cured mice that remained tumor free were rechallenged with TPSA tumors on day 68.

Immunization of mice with the LmddA-142 can control the growth and induce regression of 7-day established Tramp-C1 tumors that were engineered to express PSA in more than 60% of the experimental animals (FIG. 6B), compared to none in the untreated group (FIG. 6A). The LmddA-142 was constructed using a highly attenuated vector (LmddA) and the plasmid pADV142 (Table 1).

Further, the ability of PSA-specific CD8 lymphocytes generated by the LmddA-LLO-PSA construct to infiltrate tumors was investigated. Mice were subcutaneously implanted with a mixture of tumors and matrigel followed by two immunizations at seven day intervals with naiive or control (Lm-LLO-E7) Listeria, or with LmddA-LLO-PSA. Tumors were excised on day 21 and were analyzed for the population of CD8⁺CD62L^{low} PSA^{tetramer+} and CD4⁺ CD25⁺FoxP3⁺ regulatory T cells infiltrating in the tumors.

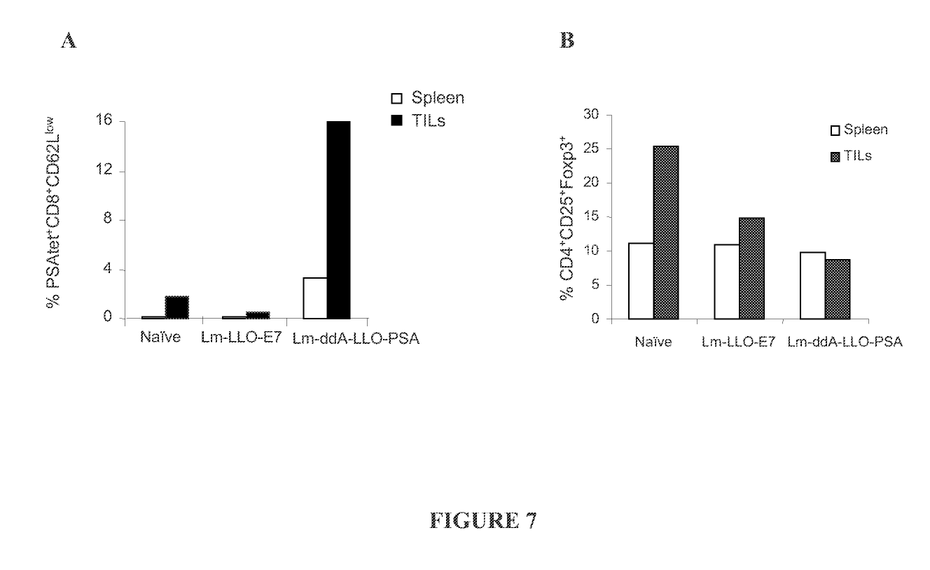

A very low number of CD8⁺CD62L^{low} PSA^{tetramer+} tumor infiltrating lymphocytes (TILs) specific for PSA that were present in the both naïve and Lm-LLO-E7 control immunized mice was observed. However, there was a 10-30-fold increase in the percentage of PSA-specific CD8⁺CD62L^{low} PSA^{tetramer+} TILs in the mice immunized with LmddA-LLO-PSA (FIG. 7A). Interestingly, the population of CD8⁺CD62L^{low} PSA^{tetramer+} cells in spleen was 7.5 fold less than in tumor (FIG. 7A).

In addition, the presence of CD4⁺/CD25⁺/Fox3⁺ T regulatory cells (regs) in the tumors of untreated mice and Listeria immunized mice was determined. Interestingly, immunization with Listeria resulted in a considerable decrease in the number of CD4⁺ CD25⁺FoxP3⁺ T-regs in tumor but not in spleen (FIG. 7B). However, the construct LmddA-LLO-PSA had a stronger impact in decreasing the frequency of CD4⁺ CD25⁺FoxP3⁺ T-regs in tumors when compared to the naïve and Lm-LLO-E7 immunized group (FIG. 7B).

Thus, the LmddA-142 vaccine can induce PSA-specific CD8⁺ T cells that are able to infiltrate the tumor site (FIG. 7A). Interestingly, Immunization with LmddA-142 was associated with a decreased number of regulatory T cells in the tumor (FIG. 7B), probably creating a more favorable environment for an efficient anti-tumor CTL activity.

### Example 7

### Lmdd-143 and LmddA -143 Secretes a Functional LLO Despite the PSA Fusion

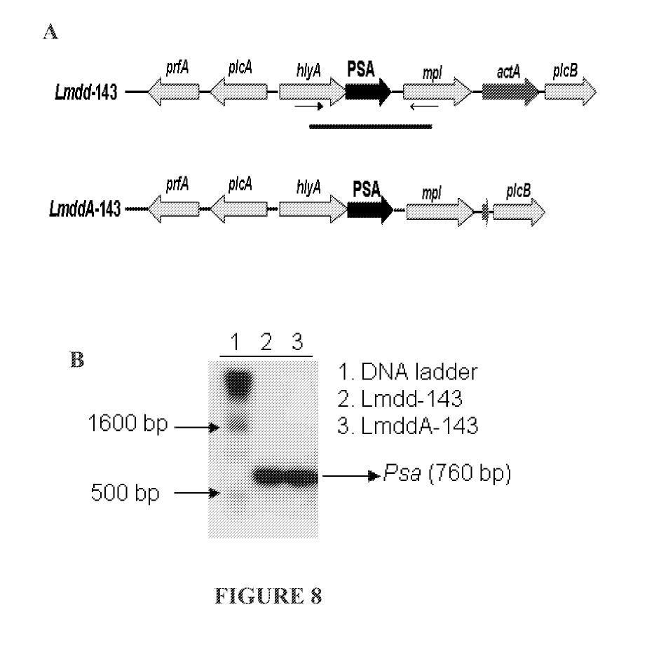

The Lmdd-143 and LmddA-143 contain the full-length human klk3 gene, which encodes the PSA protein, inserted by homologous recombination downstream and in frame with the hly gene in the chromosome. These constructs were made by homologous recombination using the pKSV7 plasmid (Smith and Youngman, Biochimie 1992; 74 (7-8) p705-711), which has a temperature-sensitive replicon, carrying the hly-klk3-mpl recombination cassette. Because of the plasmid excision after the second recombination event, the antibiotic resistance marker used for integration selection is lost. Additionally, the actA gene is deleted in the LmddA-143 strain (FIG. 8A). The insertion of klk3 in frame with hly into the chromosome was verified by PCR (FIG. 8B) and sequencing (data not shown) in both constructs.

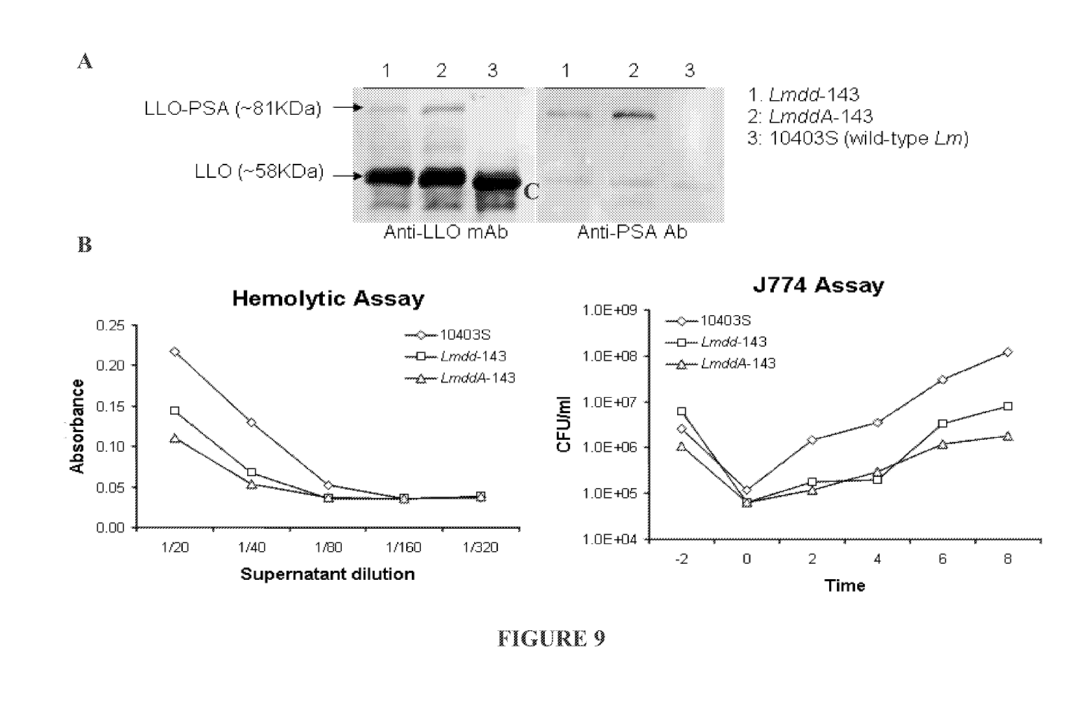

One important aspect of these chromosomal constructs is that the production of LLO-PSA would not completely abolish the function of LLO, which is required for escape of Listeria from the phagosome, cytosol invasion and efficient immunity generated by L. monocytogenes. Western-blot analysis of secreted proteins from Lmdd-143 and LmddA-143 culture supernatants revealed an ˜81 kDa band corresponding to the LLO-PSA fusion protein and an ˜60 kDa band, which is the expected size of LLO (FIG. 9A), indicating that LLO is either cleaved from the LLO-PSA fusion or still produced as a single protein by L. monocytogenes, despite the fusion gene in the chromosome. The LLO secreted by Lmdd-143 and LmddA-143 retained 50% of the hemolytic activity, as compared to the wild-type L. monocytogenes 10403S (FIG. 9B). In agreement with these results, both Lmdd-143 and LmddA-143 were able to replicate intracellularly in the macrophage-like J774 cell line (FIG. 9C).

### Example 8

### Both Lmdd-143 and LmddA-143 Elicit Cell-Mediated Immune Responses Against the PSA Antigen

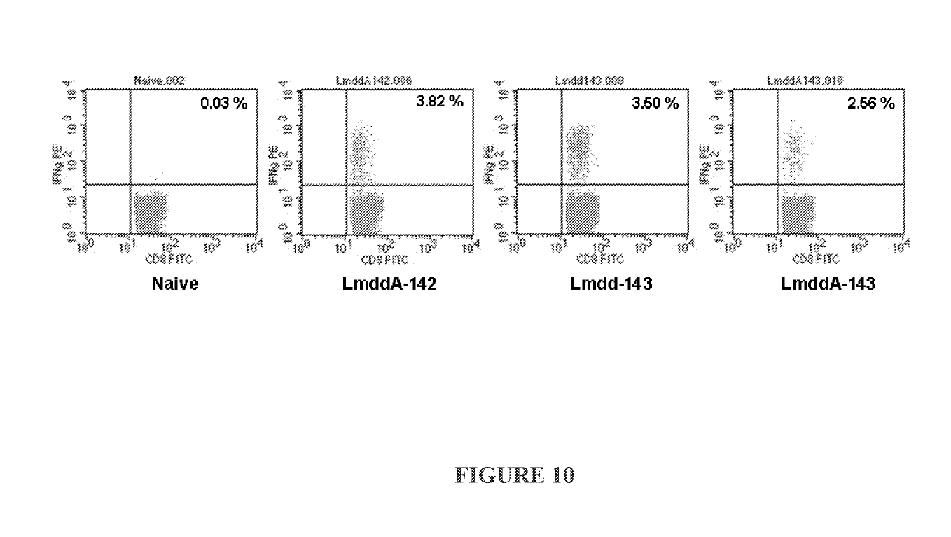

After showing that both Lmdd-143 and LmddA-143 are able to secrete PSA fused to LLO, we investigated if these strains could elicit PSA-specific immune responses in vivo. C57Bl/6 mice were either left untreated or immunized twice with the Lmdd-143, LmddA-143 or LmddA-142. PSA-specific CD8⁺ T cell responses were measured by stimulating splenocytes with the PSA₆₅₋₇₄ peptide and intracellular staining for IFN-γ. As shown in FIG. 10, the immune response induced by the chromosomal and the plasmid-based vectors is similar.

### Example 9

### A Recombinant Lm Strain Secreting a LLO-HMW-MAA Fusion Protein Results in a Broad Antitumor Response

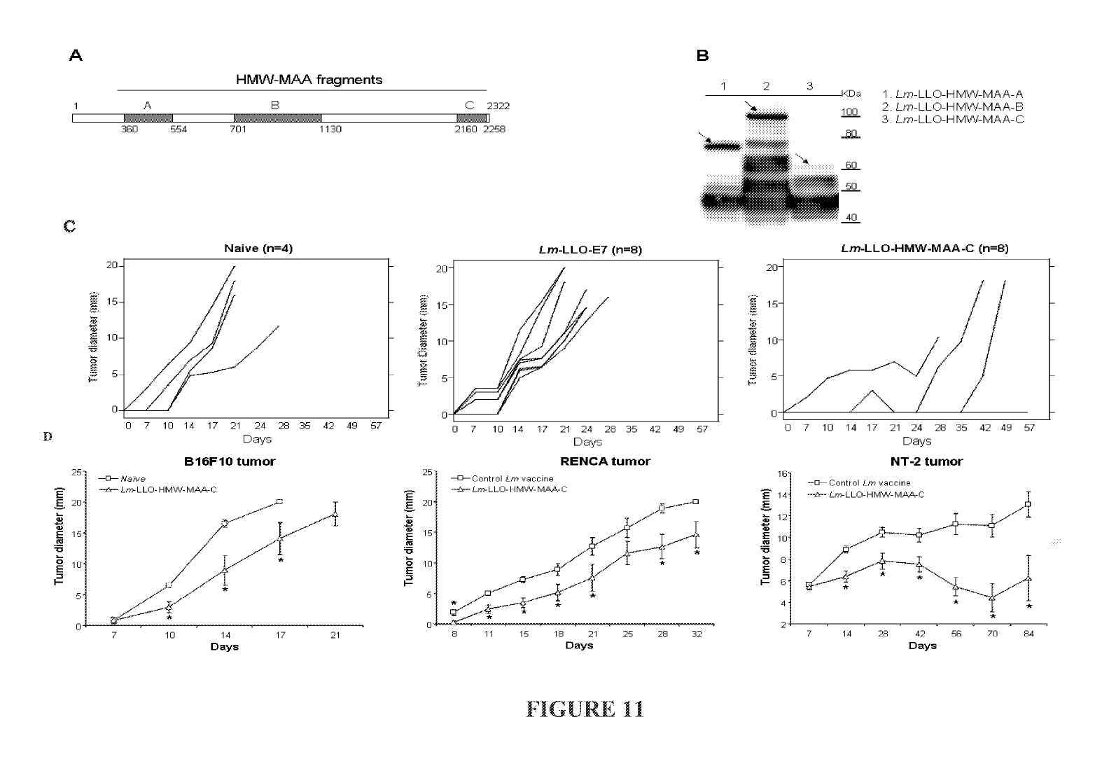

Three Lm-based vaccines expressing distinct HMW-MAA fragments based on the position of previously mapped and predicted HLA-A2 epitopes were designed (FIG. 11A). The Lm-tLLO-HMW-MMA₂₁₆₀₋₂₂₅₈ (also referred as Lm-LLO-HMW-MAA-C) is based on the avirulent Lm XFL-7 strain and a pGG55-based plasmid. This strain secretes a ˜62 kDa band corresponding to the tLLO-IIMW-MAA₂₁₆₀₋₂₂₅₈ fusion protein (FIG. 11B). The secretion of tLLO-HMW-MAA₂₁₆₀₋₂₂₅₈ is relatively weak likely due to the high hydrophobicity of this fragment, which corresponds to the HMW-MAA transmembrane domain. Using B16F10 melanoma cells transfected with the full-length HMW-MAA gene, we observed that up to 62.5% of the mice immunized with the Lm-LLO-HMW-MAA-C could impede the growth of established tumors (FIG. 11C). This result shows that HMW-MAA can be used as a target antigen in vaccination strategies. Interestingly, we also observed that immunization of mice with Lm-LLO-HMW-MAA-C significantly impaired the growth of tumors not engineered to express HMW-MAA, such as B16F10, RENCA and NT-2 (FIG. 11D), which were derived from distinct mouse strains. In the NT-2 tumor model, which is a mammary carcinoma cell line expressing the rat HER-2/neu protein and is derived from the FVB/N transgenic mice, immunization with Lm-LLO-HMW-MAA-C 7 days after tumor inoculation not only impaired tumor growth but also induced regression of the tumor in 1 out of 5 mice (FIG. 11D).

### Example 10

### Immunization of Mice with Lm-LLO-HMW-MAA-C Induces Infiltration of the Tumor Stroma by CD8⁺ T Cells and a Significant Reduction in the Pericyte Coverage in the Tumor Vasculature

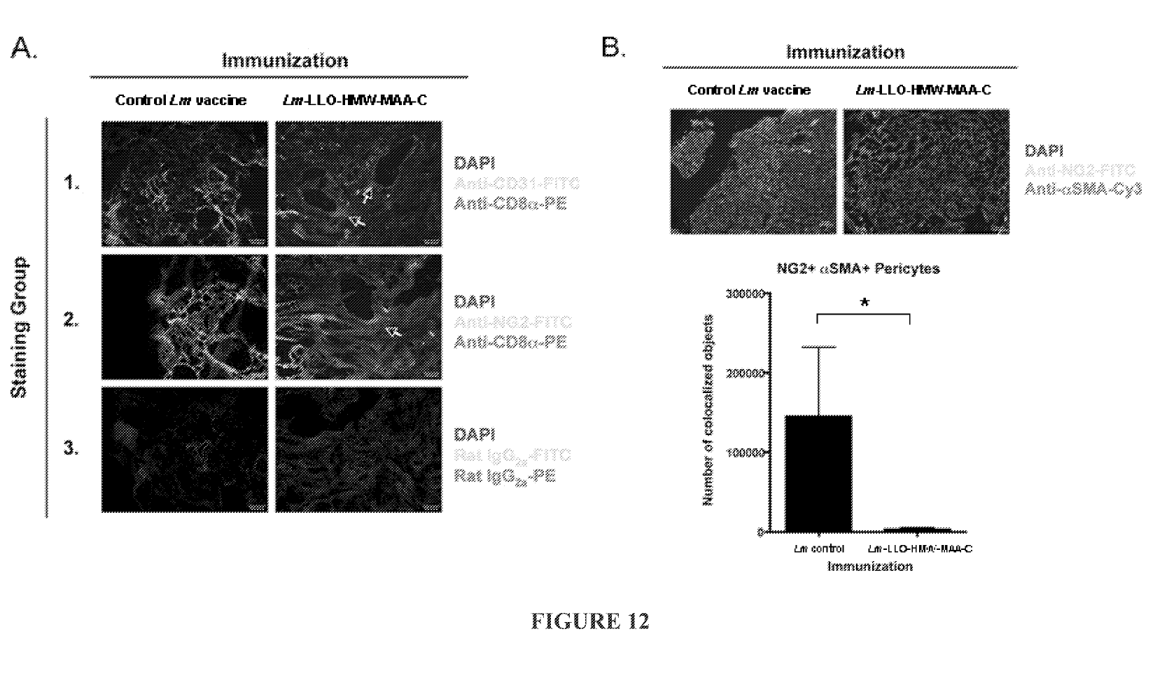

Although NT-2 cells do not express the HMW-MAA homolog NG2, immunization of FVB/N mice with Lm-LLO-HMW-MAA-C significantly impaired the growth of NT-2 tumors and eventually led to tumor regression (FIG. 11D). This tumor model was used to evaluate CD8⁺ T cells and pericytes in the tumor site by immunofluorescence. Staining of NT-2 tumor sections for CD8 showed infiltration of CD8⁺ T cells into the tumors and around blood vessels in mice immunized with the Lm-LLO-HMW-MAA-C vaccine, but not in mice immunized with the control vaccine (FIG. 12A). Pericytes in NT-2 tumors were also analyzed by double staining with αSMA and NG2 (murine homolog of HMW-MAA) antibodies. Data analysis from three independent NT-2 tumors showed a significant decrease in the number of pericytes in mice immunized with Lm-LLO-HMW-MAA-C, as compared to control (P≦0.05) (FIG. 12B). Similar results were obtained when the analysis was restricted to cells stained for αSMA, which is not targeted by the vaccine (data not shown). Thus, Lm-LLO-HMW-MAA-C vaccination impacts blood vessel formation in the tumor site by targeting pericytes.

### Example 11

### Development of a Recombinant L. monocytogenes Vector with Enhanced Anti-Tumor Activity by Concomitant Expression and Secretion of LLO-PSA and tLLO-HMW-MAA₂₁₆₀₋₂₂₅₈ Fusion Proteins, Eliciting Immune Responses to Both Heterologous Antigens

### Materials and Methods:

Construction of the pADV168 plasmid. The HMW-MAA-C fragment is excised from a pCR2.1-HMW-MAA₂₁₆₀₋₂₂₅₈ plasmid by double digestion with XhoI and XmaI restriction endonucleases. This fragment is cloned in the pADV134 plasmid already digested with XhoI and XmaI to excise the E7 gene. The pADV168 plasmid is electroporated into electrocompetent the dal⁽⁻⁾ dat⁽⁻⁾ E. coli strain MB2159 and positive clones screened for RFLP and sequence analysis.

Construction of Lmdd-143/168, LmddA-143/168 and the control strains LmddA-168, Lmdd-143/134 and LmddA-143/134. Lmdd, Lmdd-143 and LmddA-143 is transformed with either pADV168 or pADV134 plasmid. Transformants are selected on Brain-Heart Infusion-agar plates supplemented with streptomycin (250 μg/ml) and without D-alanine (BHIs medium). Individual clones are screened for LLO-PSA, tLLO-HMW-MAA₂₁₆₀₋₂₂₅₈ and tLLO-E7 secretion in bacterial culture supernatants by Western-blot using an anti-LLO, anti-PSA or anti-E7 antibody. A selected clone from each strain will be evaluated for in vitro and in vivo virulence. Each strain is passaged twice in vivo to select the most stable recombinant clones. Briefly, a selected clone from each construct is grown and injected i.p to a group of 4 mice at 1×10⁸ CFU/mouse. Spleens are harvested on days 1 and 3, homogenized and plated on BHIs-agar plates. After the first passage, one colony from each strain is selected and passaged in vivo for a second time. To prevent further attenuation of the vector, to a level impairing its viability, constructs in two vectors with distinct attenuation levels (Lmdd-143/168, LmddA-143/168) are generated.

In vitro virulence determination by intracellular replication in J774 cells. Uptake of Lm by macrophages, followed by cytosolic invasion and intracellular proliferation are required for successful antigen delivery and presentation by Lm-based vaccines. An in vitro invasion assay, using a macrophage-like J774 cell line is used to test these properties in new recombinant Lm strains. Briefly, J774 cells are infected for 1 hour in medium without antibiotics at MOI of 1:1 with either the control wild-type Lm strain 10403S or the new Lm strains to be tested. Extracellular bacteria are killed by 1 hour incubation in medium 10 μg/ml of gentamicin. Samples are harvested at regular intervals and cells lysed with water. Ten-fold serial dilutions of the lysates are plated in duplicates on BHIs plates and colony-forming units (CFU) counted in each sample.

In vivo virulence studies. Groups of four C57BL/6 mice (7 weeks old) are injected i.p. with two different doses (1×10⁸ and 1×10⁹ CFUs/dose) of Lmdd-143/168, LmddA-143/168, LmddA-168, Lmdd-143/134 or LmddA-143/134 strains. Mice are followed-up for 2 weeks for survival and LD₅₀ estimation. An LD₅₀ of >1×10⁸ constitutes an acceptable value based on previous experience with other Lm-based vaccines.

### Results

Once the pADV168 plasmid is successfully constructed, it is sequenced for the presence of the correct HMW-MAA sequence. This plasmid in these new strains express and secrete the LLO fusion proteins specific for each construct. These strains are highly attenuated, with an LD50 of at least 1×10⁸ CFU and likely higher than 1×10⁹ CFU for the actA-deficient (LmddA) strains, which lack the actA gene and consequently the ability of cell-to-cell spread. The construct is tested and the one that has a better balance between attenuation and therapeutic efficacy is selected.

### Example 12

### Detection of Immune Responses and Anti-Tumor Effects Elicited Upon Immunization with Lmdd-143/168 and LmddA -143/168

Immune responses to PSA and HMW-MAA are studied in mice upon immunization with Lmdd-143/168 and LmddA-143/168 strains using standard methods, such as detection of IFN-γ production and specific CTL activity against these antigens. The therapeutic efficacy of dual-expression vectors are tested in the TPSA23 tumor model.

Intracellular cytokine staining for IFN-γ. C57BL/6 mice (3 mice per treatment group) are immunized twice at 1-week intervals with the Lmdd-143/168 and LmddA-143/168 strains. As controls for this experiment, mice are immunized with Lmdd-143, LmddA-143, LmddA-142, LmddA-168, Lmdd-143/134, LmddA-143/134 or left untreated (naïve group). Spleens are harvested after 7 days and a single cell suspension of splenocytes are prepared. These splenocytes are plated at 2×10⁶ cells/well in a round bottom 96-well plate, in freshly prepared complete RPMI medium with IL-2 (50 U/ml) and stimulated with either the PSA H-2Db peptide, HCIRNKSVIL, (SEQ ID NO:35), or the HPV16 E7 H-2Db control peptide RAIIYNIVTF (SEQ ID NO: 36 at a final concentration of 1 μM. Since HMW-MAA-epitopes have not been mapped in the C57B1/6 mouse, HMW-MAA-specific immune responses are detected by incubating 2×10⁶ splenocytes with 2×10⁵ EL4-HMW-MAA cells. The cells are incubated for 5 hours in the presence of monensin to retain the intracellular IFN-γ in the cells. After incubation, cells are stained with anti-mouse CD8-FITC, CD3-PerCP, CD62L-APC antibodies. They are then permeabilized and stained for IFNγ-PE and analyzed in a four-color FACS Calibur (BD Biosciences).

Cytotoxicity assay. To investigate the effector activity of the PSA and HMW-MAA specific T cells generated upon vaccinations, isolated splenocytes are incubated for 5 days in complete RPMI medium containing 20 U/ml of mouse IL-2 (Sigma), in the presence of stimulator cells (mitomycin C treated MC57G cells infected with either PSA or HMW-MAA vaccinia). For the cytotoxicity assay, EL4 target cells are labeled for 15 minutes with DDAO-SE (0.6 μM) (Molecular Probes) and washed twice with complete medium. The labeled target cells are pulsed for 1 hour with either the PSA H-2Db peptide, or the HPV16 E7 H-2Db control peptide, at a final concentration of 5 μM. For HMW-MAA-specific cytotoxic responses, the EL4-HMW-MAA cells are used as targets. The cytotoxicity assay is performed for 2 hours by incubating the target cells (T) with effector cells (E) at different E:T ratios for 2-3 hours. Cells are fixed with formalin, permeabilized and stained for cleaved caspase-3 to detect induction of apoptosis in the target cells.

Anti-tumor efficacy. The anti-tumor efficacy of the Lmdd-143/168 and LmddA-143/168 strains are compared to that of LmddA-142 and LmddA-168, using the T-PSA23 tumor model (TrampC-1/PSA). Groups of 8 male C57BL/6 mice (6-8 weeks old) are inoculated s.c. with 2×10⁶ T-PSA23 cells and 7 days later immunized i.p. with 0.1×LD50 dose of Lmdd-143/168, LmddA-143/168, LmddA-142 and LmddA-168. As controls, mice are either left untreated or immunized with an Lm control strain (LmddA-134). Each group receives two additional doses of the vaccines with 7 day intervals. Tumors are monitored for 60 days or until they reach a size of 2 cm, at which point mice are sacrificed.

### Results

Immunization of mice with LmddA-168 results in the induction of specific responses against HMW-MAA. Similarly, Lmdd-143/168 and LmddA-143/168 elicits an immune response against PSA and HMW-MAA that is comparable to the immune responses generated by L. monocytogenes vectors expressing each antigen individually Immunization of T-PSA-23-bearing mice with the Lmdd-143/168 and LmddA-143/168 results in a better anti-tumor therapeutic efficacy than the immunization with either LmddA-142 or LmddA-168.

### Example 13

### Immunization with Either Lmdd-143/168 or LmddA-143/168 Results in Pericyte Destruction, Up-Regulation of Adhesion Molecules in Endothelial Cells and Enhanced Infiltration of TILs Specific for PSA

Characterization of tumor infiltrating lymphocytes and endothelial cell-adhesion molecules induced upon immunization with Lmdd-143/168 or LmddA-143/168. The tumors from mice immunized with either Lmdd-143/168 or LmddA-143/168 are analyzed by immunofluorescence to study expression of adhesion molecules by endothelial cells, blood vessel density and pericyte coverage in the tumor vasculature, as well as infiltration of the tumor by immune cells, including CD8 and CD4 T cells. TILs specific for the PSA antigen are characterized by tetramer analysis and functional tests.

Analysis of tumor infiltrating lymphocytes (TILs). TPSA23 cells embedded in matrigel are inoculated s.c in mice (n=3 per group), which are immunized on days 7 and 14 with either Lmdd-143/168 or LmddA-143/168, depending on which one is the more effective according to results obtained in anti-tumor studies. For comparison, mice are immunized with LmddA-142, LmddA-168, a control Lm vaccine or left untreated. On day 21, the tumors are surgically excised, washed in ice-cold PBS and minced with a scalpel. The tumors are treated with dispase to solubilize the Matrigel and release single cells for analysis. PSA-specific CD8⁺ T cells are stained with a PSA65-74 H-2Db tetramer-PE and anti-mouse CD8-FITC, CD3-PerCP-Cy5.5 and CD62L-APC antibodies. To analyze regulatory T cell in the tumor, TILs are stained with CD4-FITC, CD3-PerCP-Cy5.5 and CD25-APC and subsequently permeabilized for FoxP3 staining (anti-FoxP3-PE, Milteny Biotec). Cells are analyzed by a FACS Calibur cytometer and CellQuestPro software (BD Biosciences).

Immunofluorescence. On day 21 post tumor inoculation, the TPSA23 tumors embedded in matrigel are surgically excised and a fragment immediately cryopreserved in OCT freezing medium. The tumor fragments are cryosectioned for 8-10 μm thick sections. For immunofluorescence, samples are thawed and fixed using 4% formalin. After blocking, sections are stained with antibodies in blocking solution in a humidified chamber at 37° C. for 1 hour. DAPI (Invitrogen) staining are performed according to manufacturer instructions. For intracellular stains (αSMA), incubation is performed in PBS/0.1% Tween/1% BSA solution. Slides are cover-slipped using a mounting solution (Biomeda) with anti-fading agents, set for 24 hours and kept at 4° C. until imaging using Spot Image Software (2006) and BX51 series Olympus fluorescent microscope. CD8, CD4, FoxP3, αSMA, NG2, CD31, ICAM-1, VCAM-1 and VAP-1 are evaluated by immunofluorescence.

Statistical analysis: Non-parametric Mann-Whitney and Kruskal-Wallis tests are applied to compare tumor sizes among different treatment groups. Tumor sizes are compared at the latest time-point with the highest number of mice in each group (8 mice). A p-value of less than 0.05 is considered statistically significant in these analyses.

### Results

Immunization of TPSA23-bearing mice with the Lmdd-143/168 and LmddA-143/168 results in higher numbers of effector TILs specific to PSA and also decreases pericyte coverage of the tumor vasculature. Further, cell-adhesion markers are significantly up-regulated in immunized mice.

### Example 14

### Construction of an Attenuated Listeria monocytogenes Based Vaccine Expressing Mouse and Human Survivin

### Materials and Methods

Cloning of Survivin Genes in Listeria monocytogenes (LmddΔActA) Specific Plasmid

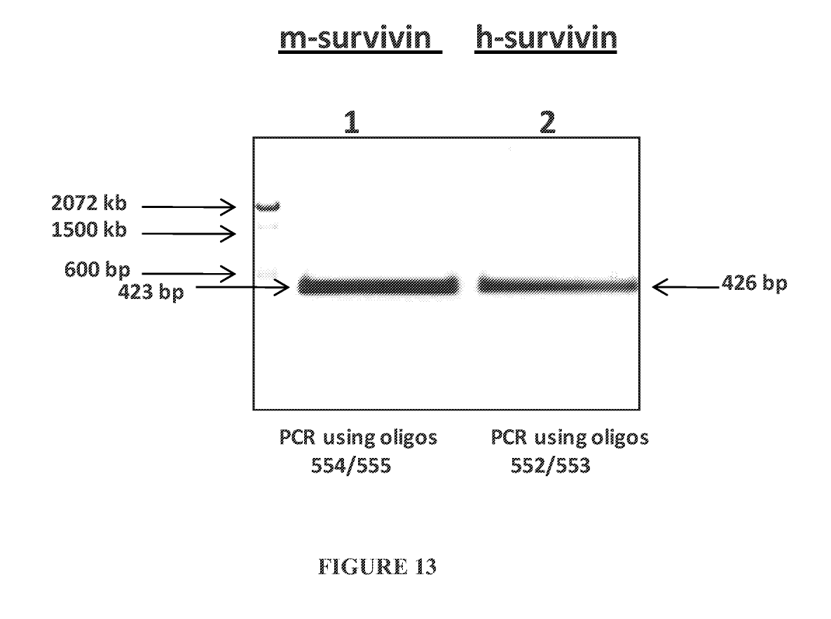

The source of survivin genes was from Dr. Don Diamond lab at City of Hope. The mouse (m-Survivin) and human Survivin (h-Survivin) DNA sequences were PCR amplified by using oligos (Adv554-atctcgagggagctccggcgctgccc (SEQ ID NO: 37 and Adv555-atcccgggttaggcagccagctgctc (SEQ ID NO: 38) for mouse survivin and oligos (Adv552-atctcgagggtgccccgacgttgccc (SEQ ID NO: 39 and Adv553-atcccggg tcaatccatggcagccagc (SEQ ID NO: 40) for human survivin fragment obtained using m-RNA sequences of the strains as template. The expected sizes of the DNA fragments after PCR amplification were 423bp for m-survivin and 426bp for h-survivin shown in FIG. 13. The fragments were purified and TA TOPO cloned into pCR2.1 plasmid resulting in the plasmids pAdv261 (m-survivin/pCR2.1) and pAdv262 (h-survivin/pCR2.1). Several h-Survivin/pCR2.1 and m-Survivin/pCR2.1 clones were PCR screened and the positive clones were confirmed by sequence verification.

Construction of Listeria monocytogenes (Lm-ddA) Vaccines

Further, pAdv261 (m-survivin/pCR2.1) and pAdv262 (h-survivin/pCR2.1) gene fragments were excised using XhoI/XmaI restriction enzymes and were cloned into pAdv142 Listeria based shuttle vector (human PSA klk3 excised from XhoI/XmaI restriction sites) resulting in the plasmids pAdv265.5 (h-Survivin/pAdv142) and pAdv266.7 (m-Survivin/pAdv142). The h-Survivin/pAdv142 and m-Survivin/pAdv142 DNA ligations were transformed into E. coli MB2159 electro-competent cells and the resulting transformants were tested for the cloning of desired gene fragment.

Human Survivin DNA sequence in plasmid pAdv265.5

(SEQ ID NO: 41)
CGGAGTGTATACTGGCTTACTATGTTGGCACTGATGAGGGTGTCAGTGAA
GTGCTTCATGTGGCAGGAGAAAAAAGGCTGCACCGGTGCGTCAGCAGAAT
ATGTGATACAGGATATATTCCGCTTCCTCGCTCACTGACTCGCTACGCTC
GGTCGTTCGACTGCGGCGAGCGGAAATGGCTTACGAACGGGGCGGAGATT
TCCTGGAAGATGCCAGGAAGATACTTAACAGGGAAGTGAGAGGGCCGCGG
CAAAGCCGTTTTTCCATAGGCTCCGCCCCCCTGACAAGCATCACGAAATC
TGACGCTCAAATCAGTGGTGGCGAAACCCGACAGGACTATAAAGATACCA
GGCGTTTCCCCCTGGCGGCTCCCTCGTGCGCTCTCCTGTTCCTGCCTTTC
GGTTTACCGGTGTCATTCCGCTGTTATGGCCGCGTTTGTCTCATTCCACG
CCTGACACTCAGTTCCGGGTAGGCAGTTCGCTCCAAGCTGGACTGTATGC
ACGAACCCCCCGTTCAGTCCGACCGCTGCGCCTTATCCGGTAACTATCGT
CTTGAGTCCAACCCGGAAAGACATGCAAAAGCACCACTGGCAGCAGCCAC
TGGTAATTGATTTAGAGGAGTTAGTCTTGAAGTCATGCGCCGGTTAAGGC
TAAACTGAAAGGACAAGTTTTGGTGACTGCGCTCCTCCAAGCCAGTTACC
TCGGTTCAAAGAGTTGGTAGCTCAGAGAACCTTCGAAAAACCGCCCTGCA
AGGCGGTTTTTTCGTTTTCAGAGCAAGAGATTACGCGCAGACCAAAACGA
TCTCAAGAAGATCATCTTATTAATCAGATAAAATATTTCTAGCCCTCCTT
TGATTAGTATATTCCTATCTTAAAGTTACTTTTATGTGGAGGCATTAACA
TTTGTTAATGACGTCAAAAGGATAGCAAGACTAGAATAAAGCTATAAAGC
AAGCATATAATATTGCGTTTCATCTTTAGAAGCGAATTTCGCCAATATTA
TAATTATCAAAAGAGAGGGGTGGCAAACGGTATTTGGCATTATTAGGTTA
AAAAATGTAGAAGGAGAGTGAAACCCATGAAAAAAATAATGCTAGTTTTT
ATTACACTTATATTAGTTAGTCTACCAATTGCGCAACAAACTGAAGCAAA
GGATGCATCTGCATTCAATAAAGAAAATTCAATTTCATCCATGGCACCAC
CAGCATCTCCGCCTGCAAGTCCTAAGACGCCAATCGAAAAGAAACACGCG
GATGAAATCGATAAGTATATACAAGGATTGGATTACAATAAAAACAATGT
ATTAGTATACCACGGAGATGCAGTGACAAATGTGCCGCCAAGAAAAGGTT
ACAAAGATGGAAATGAATATATTGTTGTGGAGAAAAAGAAGAAATCCATC
TGAATGCAATTTCGAGCCTAACCTATCCAGGTGCTCTCGTAAAAGCGAAT
AATCAAAATAATGCAGACATTCAAGTTGTCGGAATTAGTAGAAAATCAAC
CAGATGTTCTCCCTGTAAAACGTGATTCATTAACACTCAGCATTGATTTG
CCAGGTATGACTAATCAAGACAATAAAATAGTTGTAAAAAATGCCACTAA
ATCAAACGTTAACAACGCAGTAAATACATTAGTGGAAAGATGGAATGAAA
AATATGCTCAAGCTTATCCAAATGTAAGTGCAAAAATTGATTATGATGAC
GAAATGGCTTACAGTGAATCACAATTAATTGCGAAATTTGGTACAGCATT
TAAAGCTGTAAATAATAGCTTGAATGTAAACTTCGGCGCAATCAGTGAAG
GGAAAATGCAAGAAGAAGTCATTAGTTTTAAACAAATTTACTATAACGTG
AATGTTAATGAACCTACAAGACCTTCCAGATTTTTCGGCAAAGCTGTTAC
TAAAGAGCAGTTGCAAGCGCTTGGAGTGAATGCAGAAAATCCTCCTGCAT
ATATCTCAAGTGTGGCGTATGGCCGTCAAGTTTATTTGAAATTATCAACT
AATTCCCATAGTACTAAAGTAAAAGCTGCTTTTGATGCTGCCGTAAGCGG
AAAATCTGTCTCAGGTGATGTAGAACTAACAAATATCATCAAAAATTCTT
CCTTCAAAGCCGTAATTTACGGAGGTTCCGCAAAAGATGAAGTTCAAATC
ATCGACGGCAACCTCGGAGACTTACGCGATATTTTGAAAAAAGGCGCTAC
TTTTAATCGAGAAACACCAGGAGTTCCCATTGCTTATACAACAAACTTCC
TAAAAGACAATGAATTAGCTGTTATTAAAAACAACTCAGAATATATTGAA
ACAACTTCAAAAGCTTATACAGATGGAAAAATTAACATCGATCACTCTGG
AGGATACGTTGCTCAATTCAACATTTCTTGGGATGAAGTAAATTATGATC
TCGAGGGTGCCCCGACGTTGCCCCCTGCCTGGCAGCCCTTTCTCAAGG
ACCACCGCATCTCTACATTCAAGAACTGGCCCTTCTTGGAGGGCTGCGC
CTGCGCCCCGGAGCGGATGGCCGAGGCTGGCTTCATCCACTGCCCCACT
GAGAACGAGCCAGACTTGGCCCAGTGTTTCTTCTGCTTCAAGGAGCTGG
AAGGCTGGGAGCCAGATGACGACCCCATAGAGGAACATAAAAAGCATTC
GTCCGGTTGCGCTTTCCTTTCTGTCAAGAAGCAGTTTGAAGAATTAACC
CTTGGTGAATTTTTGAAACTGGACAGAGAAGAGCCAAGAACAAATTGCA
AAGGAAACCAACAATAAGAAGAAGAATTTGAGGAAACTGCGAAGAAAGT
GCGCCGTGCCATCGAGCAGCTGGCTGCCATGGATTGACCCGGGCCACT
AACTCAACGCTAGTAGTGGATTTAATCCCAAATGAGCCAACAGAACCAG
AACCAGAAACAGAACAAGTAACATTGGAGTTAGAAATGGAAGAAGAAAA
AAGCAATGATTTCGTGTGAATAATGCACGAAATCATTGCTTATTTTTTT
AAAAAGCGATATACTAGATATAACGAAACAACGAACTGAATAAAGAATA
CAAAAAAAGAGCCACGACCAGTTAAAGCCTGAGAAACTTTAACTGCGAG
CCTTAATTGATTACCACCAATCAATTAAAGAAGTCGAGACCCAAAATTT
GGTAAAGTATTTAATTACTTTATTAATCAGATACTTAAATATCTGTAAA
CCCATTATATCGGGTTTTTGAGGGGATTTCAAGTCTTTAAGAAGATACC
AGGCAATCAATTAAGAAAAACTTAGTTGATTGCCTTTTTTGTTGTGATT
CAACTTTGATCGTAGCTTCTAACTAATTAATTTTCGTAAGAAAGGAGAA
CAGCTGA

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

aaaataatctcgca
aaatatccgagaatattttggaaagtctttgccagttgatctaacg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

TGAAAATAAAACCCGCACTATGCCATTACA
TTTATATCTATGATACGTGTTTGTTTTTCTTTGCTGGCTAGCttaattgc
ttatatttacctgcaataaaggatacttacaccattatactcccattacc
aaaaacatacggggaacacgggaacttattgtacaggccacctcatagtt
aatggatcgagccacctgcaatctcatccatggaaatatattcatccccc
tgccggcctattaatgtgacttagtgcccggcggatattcctgatccagc
tccaccataaattggtccatgcaaattcggccggcaattacaggcgattc
ccttcacaaggatgtcggtccattcaattacggagccagccgtccgcata
gcctacaggcaccgtcccgatccatgtgtattaccgctgtgtactcggct
ccgtagctgacgctctcgccattctgatcagatgacatgtgacagtgtcg
aatgcagggtaaatgccggacgcagctgaaacggtatctcgtccgacatg
tcagcagacgggcgaaggccatacatgccgatgccgaatctgactgcatt
aaaaaagccattacagccggagtccagcggcgctgacgcgcagtggacca
ttagattattaacggcagcggagcaatcagctattaaagcgctcaaactg
cattaagaaatagcctctactattcatccgctgtcgcaaaatgggtaaat
accccatgcactttaaacgagggagcggtcaagaattgccatcacgactg
aacttcacctctgatttacaccaagtctgacatccccgtatcgaccacag
atgaaaatgaagagaaccattacgtgtggcgggctgcctcctgaagccat
tcaacagaataacctgttaaggtcacgtcatactcagcagcgattgccac
atactccgggggaaccgcgccaagcaccaatataggcgcatcaatcccta
ttgcgcagtgaaatcgcttcatccaaaatggccacggccaagcatgaagc
acctgcgtcaagagcagcctttgctgtttctgcatcaccatgcccgtagg
cgtagcatcacaactgccatcaagtggacatgacaccgatatgattacat
attgctgacattaccatatcgcggacaagtcaatttccgcccacgtatct
ctgtaaaaaggattgtgctcatggaaaactcctctattatcagaaaatcc
cagtacgtaattaagtatttgagaattaaattatattgattaatactaag
atacccagattcacctaaaaaacaaatgatgagataatagctccaaaggc
taaagaggactataccaactatttgttaATTAA.
Legend key:
-Normal UPPERCASE sequences : hly promoter.
-Italicized UPPERCASE sequences: p15 origin.
-Bolded UPPERCASE sequences: t-LLO ORF.
-Italicized and underlined UPPERCASE sequences: survivin.
-Italicized, bolded and underlined UPPERCASE sequences: Rep R ORF.
-Lower case sequences: P60-Bacillus dal.

The list of oligos and the DNA regions that were sequenced for the plasmids pAdv265.5 and pAdv266.7 is given in the table below

| | DNA region |
|---|---|
| Oligos number | sequenced |
| For mouse | |
| survivin | |
| Adv16 | 2393-3242 |
| Adv555 | 1865-2771 |
| For human | |
| survivin | |
| Adv16 | 2353-3261 |
| Adv553 | 1874-2796 |

### Expression and Secretion of LLO-Survivin Fusion Protein

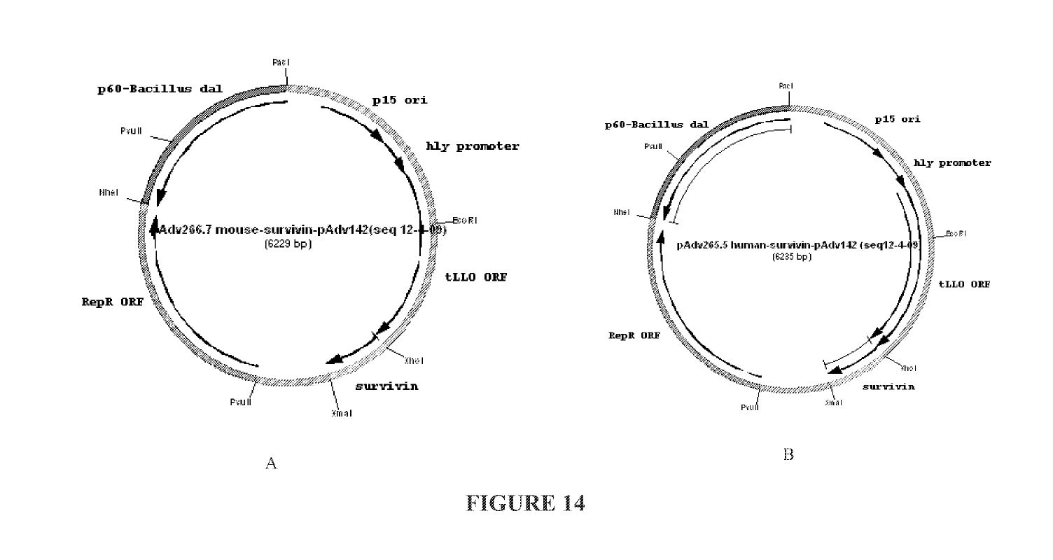

The new plasmids, h-Survivin/pAdv142 (pAdv 265.5) (FIG. 14B) and m-Survivin/pAdv142 (pAdv 266.7) (FIG. 14A) were transformed into Listeria LmddA backbone. Several Listeria clones named as LmddA-265.5 (h-Suvivin/pAdv142) and LmddA-266.7 (m-Survivin/pAdv142) were selected and screened for the expression and secretion of chromosomal LLO protein detected using the monoclonal antibody anti-B3-19, truncated LLO-Survivin fusion protein and disintegrated t-LLO protein detected using polyclonal antibody anti-PEST and as well as tLLO-Survivin fusion protein detected using the monoclonal antibody anti-Survivin. Clone#1 from LmddA-265.5 (h-Suvivin/pAdv142) and LmddA-266.7 (m-Survivin/pAdv142) constructs were selected for the first in vivo passage.

### Example 15

### In vivo Passaging of the Strain Lm-ddA-LLO-Survivin

Expression of Lm-ddA-LLO-Survivin After Two in vivo Passages

LmddA-265.5 (h-Suvivin/pAdv142) and LmddA-266.7 (m-Survivin/pAdv142) stocks were prepared for the first in vivo passage. For in vivo passaging (P1), one mouse was administered with 10⁸ CFU of each construct intraperitoneally and mouse spleens were harvested day 1 post-injection. The total number of colonies that were recovered in the spleen is indicated below.

LmddA-265.5 (h-Suvivin/pAdv142)=9.8×10³ CFU/spleen.

LmddA-266.7 (m-Survivin/pAdv142) =1.42×10⁴ CFU/spleen.

LmddA-265.5 (h-Suvivin/pAdv142) and LmddA-266.7 (m-Survivin/pAdv142) stocks were prepared for the second in vivo passage. For second in vivo passage (P2), one mouse was injected intraperitoneally with 10⁸ CFU of each construct and mouse spleen was harvested on day 1 post-injection. The total number of colonies that were recovered in the spleen is indicated below.

LmddA-265.5 (h-Suvivin/pAdv142)=1.40×10⁴ CFU/spleen.

LmddA-266.7 (m-Survivin/pAdv142)=6.38×10⁴ CFU/spleen.

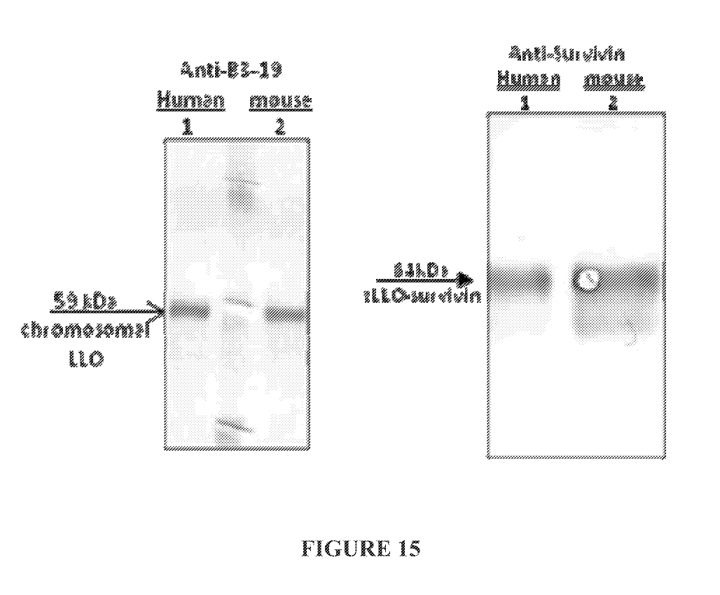

Three colonies from LmddA-265.5 (h-Survivin/pAdv142) and LmddA-266.7 (m-Survivin/pAdv142) (P2, Day 1) were selected for protein expression. All three colonies from these constructs retained expression and secretion of the tLLO-Survivin fusion protein after the second in vivo passage, as detected by immunoblotting using the monoclonal antibody specific for Survivin (FIG. 15).

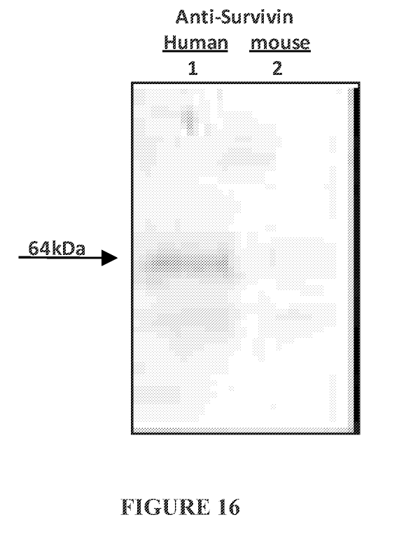

These constructs LmddA-265.5 (human-Survivin/pAdv142) and LmddA-266.7 (mouse-Survivin/pAdv142) retained the expression and secretion of the tLLO-Survivin fusion protein after two in vivo passages in C57BL/6 mice (FIG. 16).

### Example 16

### Reduction of Tumor Growth After Treatment with Listeria-Based Immunotherapy Expressing Survivin

In this study, mice were implanted with 1×10⁶ NT-2 tumors on Day 0 and treated with 2×10⁸ CFU of LmddA265.5 (survivin) immunotherapy on days 6, 13 and 20. The tumor growth was measured by calipers and study was terminated on day 65. This data provides evidence that LmddA265.5 impacts on the growth of established NT2 tumors in FvB mouse. Treatment with LmddA265.5 caused stabilization of tumor growth in mice bearing NT2 tumors and it was observed till day 65 (see FIG. 17).

Having described embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A recombinant nucleic acid molecule comprising an open reading frame encoding a recombinant polypeptide, said recombinant polypeptide comprising a heterologous antigen fused to an N-terminal Listeriolysin O (LLO) polypeptide, wherein said heterologous antigen is survivin, wherein said nucleic acid further comprises a gram-negative origin of replication sequence operably linked to a first promoter sequence, a gram-positive origin of replication sequence, and an open reading frame encoding a metabolic enzyme operably linked to a second promoter sequence.

2. The recombinant nucleic acid molecule of any one of claim 1, wherein said nucleic acid molecule is a DNA plasmid.

3. The recombinant nucleic acid molecule of any one of claims 1-2, wherein said nucleic acid molecule comprises SEQ ID NO: 41.

4. The recombinant nucleic acid molecule of any one of claims 1-3, wherein said gram-negative origin of replication sequence is a p15 sequence.

5. The recombinant nucleic acid molecule of any one of claims 1-4, wherein said gram-positive origin of replication sequence is a Rep R sequence.

6. The recombinant nucleic acid molecule of any one of claims 1-5, wherein said first promoter sequence is an hly promoter sequence.

7. The recombinant nucleic acid molecule of any one of claims 1-6, wherein said metabolic enzyme is a D-alanine racemase enzyme.

8. The recombinant nucleic acid molecule of any one of claims 1-7, wherein said second promoter sequence is a P60 promoter sequence.

9. A recombinant Listeria comprising the nucleic acid molecule of claims 1-8.

10. The recombinant Listeria of claim 9, wherein said Listeria comprises a mutation in the endogenous dal/dat genes.

11. The recombinant Listeria of claims 8-10, wherein said Listeria comprises a mutation in the endogenous actA gene.

12. The recombinant Listeria of claims 8-11, wherein said mutation is a deletion or an inactivation.

13. The recombinant Listeria strain of claims 9-12, wherein said recombinant Listeria strain is capable of escaping the phagolysosome.

14. The recombinant Listeria strain of claims 9-13, wherein said dal/dat mutation is complemented by said metabolic enzyme encoded by said nucleic acid molecule.

15. The recombinant Listeria strain of claims 9-14, wherein said recombinant Listeria strain has been passaged through an animal host.

16. The recombinant Listeria strain of claims 9-15, wherein said recombinant Listeria strain is a recombinant Listeria monocytogenes strain.

17. An immunogenic composition comprising the recombinant Listeria strain of claims 9-16 and an adjuvant, cytokine, chemokine, or combination thereof.

18. A method of inducing an anti-survivin immune response in a subject the method comprising administering the recombinant Listeria of claims 9-16, or the immunogenic composition of claim 17.

19. The method of claim 18, wherein said recombinant Listeria strain or said immunogenic composition is administered orally, or intravenously.

20. A method of treating, suppressing, or inhibiting a tumor or cancer in a subject comprising administering the recombinant Listeria of claims 9-16, or the immunogenic composition of claim 15.

21. The method of claim 20, wherein said tumor or cancer is breast tumor or cancer, ovarian tumor or cancer, brain tumor or cancer, lung tumor or cancer, gastrointestinal tumor or cancer, sarcoma, pancreatic tumor or cancer, a lymphoma or a combination thereof.

22. The method of claims 20-21, further comprising the step of administering a booster dose of said recombinant Listeria or said immunogenic composition or an alternate form thereof.

23. The method of claim 22, wherein said alternate form of said immunogenic composition is a DNA vaccine encoding a recombinant polypeptide comprising a survivin antigen fused to an N-terminal Listeriolysin O (LLO) polypeptide, a N-terminal ActA polypeptide, or a PEST-peptide, a recombinant polypeptide comprising said antigen fused to an N-terminal Listeriolysin O (LLO) polypeptide, a N-terminal ActA polypeptide, or a PEST-peptide, or a viral vector encoding said recombinant polypeptide.

24. Use of the recombinant Listeria or immunogenic composition of any one of claims 9-17 for inducing an anti-survivin immune response in a subject or for treating, suppressing, or inhibiting a survivin-expressing cancer in a subject, or for treating, suppressing, or inhibiting a survivin-expressing tumor in a subject.

25. The use of claim 24 wherein said Listeria or said immunogenic composition is administered orally, or intravenously.

26. The use of claim 25, wherein said tumor or cancer is breast tumor or cancer, ovarian tumor or cancer, brain tumor or cancer, lung tumor or cancer, gastrointestinal tumor or cancer, sarcoma, pancreatic tumor or cancer, a lymphoma or a combination thereof.
